(19) United States
(12) Patent Application Publication
(10) Pub. No.: US 2026/0297008 A1
(43) Pub. Date: Oct. 1, 2026

(54) REDUCED COENZYME Q10 COMPOSITION AND PRODUCTION METHOD FOR THE SAME

(71) Applicant: KANEKA CORPORATION, Osaka (JP)

(72) Inventors: Subaru Tanizaki, Hyogo (JP); Tsuyoshi Matsuno, Hyogo (JP); Takao Yamaguchi, Hyogo (JP)

(73) Assignee: KANEKA CORPORATION, Osaka (JP)

(21) Appl. No.: 19/659,329

(22) Filed: Apr. 27, 2026

Related U.S. Application Data
(63) Continuation of application No. PCT/JP2025/045817, filed on Dec. 26, 2025.

(30) Foreign Application Priority Data
Mar. 31, 2025 (JP) 2025-058008
Mar. 31, 2025 (JP) 2025-058032
Mar. 31, 2025 (JP) 2025-058139

(51) Int. Cl. C07C 49/84; C07C 45/81; C07C 45/86

(52) U.S. Cl. CPC C07C 49/84; A61K 31/09; C07C 45/81; C07C 45/86

(57) ABSTRACT

A reduced coenzyme Q10 composition in which transition to other crystal forms is unlikely is provided. The reduced coenzyme Q10 composition includes reduced coenzyme Q10, reduced demethoxy Q10, and reduced coenzyme Q9. The peak area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 in a chromatogram of the composition is 0.3 or more.

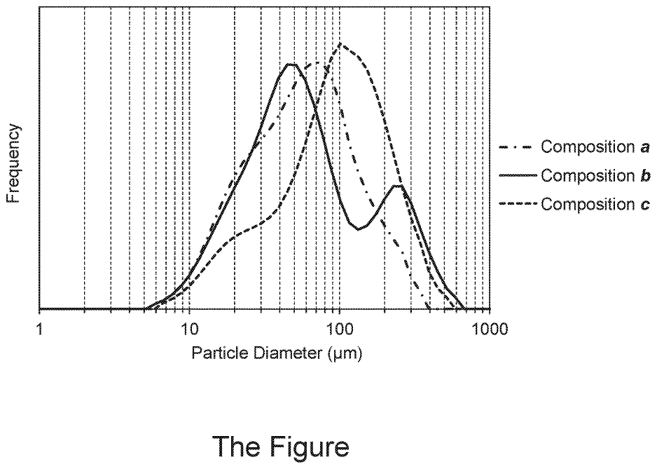

### TECHNICAL FIELD

One or more embodiments of the present invention relate to a reduced coenzyme Q10 composition and a production method for the same.

### BACKGROUND

Coenzyme Q is an essential component that is widely distributed in living organisms from bacteria to mammals, and is known as a component of the mitochondrial electron transport chain in cells in vivo. In humans, coenzyme Q10, in which the side chain of coenzyme Q has a repeating structure of 10 units, is the main component, and, in vivo, typically, about 40 to 90% of coenzyme Q10 is present in the reduced form. Examples of physiological actions of coenzyme Q include activation of energy production through mitochondrial activation action, activation of cardiac function, a stabilization effect of cell membranes, a protective effect on cells through anti-oxidation action, and the like.

Although many of the coenzyme Q10 currently manufactured and sold are oxidized coenzyme Q10 (hereinafter sometimes referred to as “QX”), in recent years reduced coenzyme Q10 (hereinafter sometimes referred to as “QH”), which exhibits higher oral absorbability compared to oxidized coenzyme Q10, has also appeared on the market and is being used.

Since oxidation easily occurs in reduced coenzyme Q10, the storage cost is high and the application range of product forms is limited. In particular, a composition in solid form that contains reduced coenzyme Q10, has excellent oxidative stability, and is applicable to forms such as granules, hard capsules, and tablets is desired.

On the other hand, with regard to methods for obtaining reduced coenzyme Q10 as a crystal, several methods are also known. For example, in Patent Literature 1, a method that crystallizes reduced coenzyme Q10 in an alcohol solution and/or in a ketone solution and produces a crystal is disclosed. Further, in Patent Literature 2, crystallization is carried out by adding a high-concentration liquid phase of reduced coenzyme Q10 to a poor solvent.

Further, Patent Literature 3 discloses that crystal polymorphism is observed in reduced coenzyme Q10, and that a specific crystal form within this crystal polymorphism (hereinafter, this crystal is referred to as “Form II crystal”) is more stable than a conventional reduced coenzyme Q10 (hereinafter, this crystal is referred to as “Form I crystal”), and it is reported that other physical properties are also different from those of the Form I crystal. The Form I crystal is a relatively unstable metastable form as compared to the newly discovered Form II crystal.

### PATENT LITERATURE

Patent Literature 1: WO 2003/006409 A
Patent Literature 2: JP Patent Publication No. 2003-089669 A
Patent Literature 3: WO 2012/176842 A

In the case of a crystal form other than the stable form, for example, during operations such as crystallization, drying, pulverization, or the like, there is a possibility that a transition to the stable form may occur. Although this is a very natural phenomenon in which a substance attempts to undergo a change to an energetically stable state, such a transition can result in changes in the physical properties of the resulting crystal, there is a possibility that quality variations occur in the crystal and in a composition in which the crystal is an active ingredient. In pharmaceutical and functional food and the like, crystal polymorphism should be strictly controlled, and with respect to a composition containing reduced coenzyme Q10 as well, it is desirable to control crystal polymorphism.

### SUMMARY

Accordingly, in view of the above, a reduced coenzyme Q10 composition in which transition to other crystal forms is unlikely and a production method for the same are provided.

The present inventors have found that, by further causing a composition containing reduced coenzyme Q10 to contain analogs having a chemical structure similar to that of reduced coenzyme Q10 and by controlling the peak area ratio in a chromatogram among the plurality of analogs relative to each other to be within a specific range, a transition of reduced coenzyme Q10 to the other crystal form is suppressed, thereby improving stability, and have completed one or more embodiments of the present invention.

That is, the gist of the first aspect of one or more embodiments of the present invention is as follows.
(1) A reduced coenzyme Q10 composition comprising:
   reduced coenzyme Q10,
   reduced demethoxy Q10, and
   reduced coenzyme Q9,
   wherein, in a chromatogram of the composition obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 mL/min, and
   a detection wavelength: 190 nm,
   wherein an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 is 0.3 or more.
(2) The reduced coenzyme Q10 composition according to the above (1),
   wherein, in the chromatogram of the composition, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 is 1.0 or more.
(3) The reduced coenzyme Q10 composition according to the above (1),
   wherein, in the chromatogram of the composition, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 is 2.0 or more.
(4) The reduced coenzyme Q10 composition according to the above (1),
   wherein the reduced coenzyme Q10 composition is in solid form.
(5) The reduced coenzyme Q10 composition according to the above (1), further comprising an antioxidant.
(6) A production method of reduced coenzyme Q10 composition comprising:
   a step of separating, from a solution containing reduced coenzyme Q10, reduced demethoxy Q10, and reduced coenzyme Q9, a composition in solid form containing the reduced coenzyme Q10, the reduced demethoxy Q10, and the reduced coenzyme Q9,
   wherein, in a chromatogram of the composition after the separation obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 mL/min, and
   a detection wavelength: 190 nm,
   wherein an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 is 0.3 or more.
(7) The production method for the reduced coenzyme Q10 composition according to the above (6),
   wherein the step of separating includes adding a seed crystal of reduced coenzyme Q10 to the solution and cooling the solution.

According to the first aspect of one or more embodiments of the present invention, a reduced coenzyme Q10 composition in which polymorphic transition of reduced coenzyme Q10 is unlikely to occur and which has high stability can be obtained.

### BRIEF DESCRIPTION OF DRAWINGS

The FIGURE is a particle size distribution on a volume basis of particles in Compositions a, b, and c, measured by laser diffraction/scattering method under wet conditions.

### DETAILED DESCRIPTION

Hereinafter, the aspects of one or more embodiments of the present invention will be described in detail based on embodiments.

### <<First Aspect (Disclosure of Japanese Patent Application No. 2025-058032)>>

The reduced coenzyme Q10 composition of one or more embodiments of the present invention is characterized in that the reduced coenzyme Q10 composition contains reduced coenzyme Q10, reduced demethoxy Q10, and reduced coenzyme Q9.

### <Coenzyme Q10>

The reduced coenzyme Q10 composition according to the present embodiment contains, as a main component, reduced coenzyme Q10 having the following chemical structure. In addition, the reduced coenzyme Q10 composition may contain oxidized coenzyme Q10 having the following chemical structure. Here, the main component means that, with respect to the total weight of coenzyme Q10 in the composition, the proportion of reduced coenzyme Q10 contained is, for example, 50 wt % or more, typically 60 wt % or more, preferably 70 wt % or more, more preferably 80 wt % or more, still more preferably 90 wt % or more, particularly preferably 95 wt % or more, and especially 98 wt % or more.

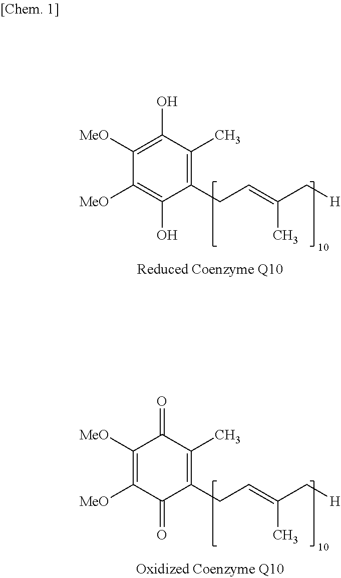

Reduced coenzyme Q10 can be obtained, for example, by known methods such as synthesis, fermentation, and extraction from a natural product, as well as by combining those with a reduction reaction as necessary. Preferably, it can be obtained by reducing oxidized coenzyme Q10, such as existing high-purity coenzyme Q10, or a mixture of oxidized coenzyme Q10 and reduced coenzyme Q10, using a general reducing agent, for example, sodium dithionite, sodium borohydride, ascorbic acids and the like.

In the present embodiment, the reduced coenzyme Q10 in the composition may be contained in the state of a crystal, or may be contained in the state of a crystalline solid. In the present specification, “crystalline solid” means a solid that contains therein, together with a portion having a crystal structure, an amorphous component that does not have a crystal structure. Specifically, in order to be a crystalline solid, with respect to the total weight of QH, the crystal is preferably 80 wt % or more, more preferably 90 wt % or more, even more preferably 95 wt % or more, and most preferably 98 wt % or more. Note that the degree of crystallization of each component such as reduced coenzyme Q10 and the like contained in the composition can be calculated, in accordance with the following formula, from the theoretical melting heat obtained from the content of each component such as reduced coenzyme Q10 and the like in the composition and from the measured melting heat data obtained by measuring the crystal melting heat by DSC analysis.

$$Degree of crystallization ( \% ) = ( measured melting enthalpy / theoretical melting enthalpy ) \times 100$$

In reduced coenzyme Q10, there are two crystal polymorphs, the Form I type and the Form II type, and both are applicable. Specifically, the crystal form of reduced coenzyme Q10 having a melting point around 48° C. and showing characteristic peaks at diffraction angles (2θ±0.2°) of 3.1°, 18.7°, 19.0°, 20.2°, and 23.0° in powder X-ray (Cu-Kα) diffraction is a Form I crystal, and the crystal form of reduced coenzyme Q10 having a melting point around 52° C. and showing characteristic peaks at diffraction angles (2θ±0.2°) of 11.5°, 18.2°, 19.3°, 22.3°, 23.0°, and 33.3° in powder X-ray (Cu-Kα) diffraction is a Form II crystal.

In one or more embodiments of the present invention, a composition can contain, as reduced coenzyme Q10 (QH), Form I crystal and Form II crystal of reduced coenzyme Q10 at any blending proportion. For example, with respect to the total of Form I crystal and Form II crystal, the proportion of Form I crystal can be 90 wt % or more, in one embodiment, 95 wt % or more, in one embodiment, 98 wt % or more, and in one embodiment, 100 wt % or less. For example, with respect to the total of the Form I crystal and the Form II crystal, the proportion of the Form I crystal can be 90 to 100 wt %, preferably 95 to 100 wt %, particularly preferably 98 to 100 wt %. The reduced coenzyme Q10 composition of the present embodiment, because a transition of QH to other crystal forms is unlikely to occur, can maintain over a long period the properties and quality of the composition at the initial time of manufacture.

In a reduced coenzyme Q10 composition according to one or more embodiments of the present invention, the content of reduced coenzyme Q10 in the composition (weight of reduced coenzyme Q10/total weight of the composition) is not particularly limited, but is typically 0.1 wt % or more, preferably 0.5 wt % or more, more preferably 1 wt % or more, particularly preferably 2 wt % or more, and still more preferably 5 wt % or more. The upper limit is not particularly limited, but is 50 wt % or less, preferably 49 wt % or less, more preferably 48 wt % or less, particularly preferably 47 wt % or less, and further preferably 46 wt % or less. That is, the content of reduced coenzyme Q10 in the composition can be within the range of 0.1 to 50 wt %, preferably 0.5 to 49 wt %, more preferably 1 to 48 wt %, particularly preferably 2 to 47 wt %, and still more preferably 5 to 46 wt %.

In a reduced coenzyme Q10 composition according to one or more embodiments of the present invention, the content of reduced coenzyme Q10 in the composition (reduced coenzyme Q10 weight/total composition weight) is typically 51 wt % or more, preferably 56 wt % or more, more preferably 61 wt % or more, particularly preferably 66 wt % or more, and still more preferably 71 wt % or more. The upper limit is not particularly limited, but is 99 wt % or less, preferably 98 wt % or less, more preferably 97 wt % or less, particularly preferably 96 wt % or less, and even more preferably 95 wt % or less. That is, the content of reduced coenzyme Q10 in the composition can be within the range of 51 to 99 wt %, preferably 56 to 98 wt %, more preferably 61 to 97 wt %, particularly preferably 66 to 96 wt %, and still more preferably 71 to 95 wt %.

The reduced coenzyme Q10 composition according to the present embodiment contains, in addition to the above reduced coenzyme Q10, reduced demethoxy Q10 (which may hereinafter be referred to as “RD”) and reduced coenzyme Q9 (which may hereinafter be referred to as “RQ9”), having the following chemical structure.

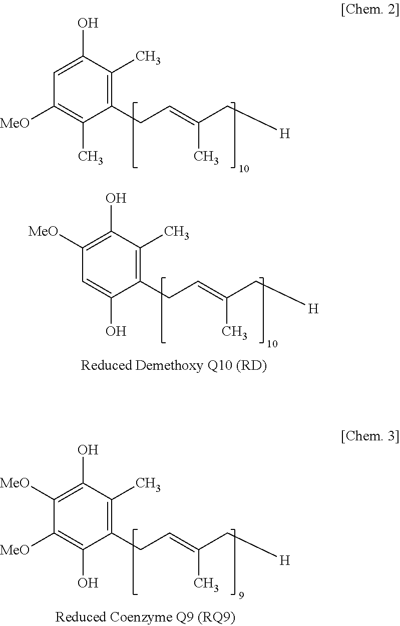

Reduced demethoxy Q10 (RD) corresponds to a compound in which the methoxy group in QH is missing, and reduced coenzyme Q9 (RQ9) corresponds to a compound in which the isoprene chain in QH is shorter by one unit. Note that, in the present specification, “reduced demethoxy Q10 (RD)” also includes analogs of reduced demethoxy Q10. Examples of the analogs of the reduced demethoxy Q10 include compounds in which the isoprene chain of the reduced demethoxy Q10 has undergone cyclization at one site or double-bond formation at one site, compounds in which cyclization has occurred at one site or at two or more sites of the isoprene chain of QH, and compounds in which the formation of a double bond has occurred at one site or at two or more sites of the isoprene chain of QH. These RD and RQ9, as with QH, can be obtained by known methods such as synthesis, fermentation, and extraction from natural products, as well as by combining those with a reduction reaction, as necessary. Preferably, RD and RQ9, that are generated together as by-products when reduced coenzyme Q10 is produced by reducing a composition containing existing oxidized coenzyme Q10 with a general reducing agent, for example, sodium dithionite, sodium borohydride, ascorbic acids, or the like, may be used as they are.

In the present embodiment, RD and RQ9 in the composition may be contained in the crystal state, or may be contained in the crystalline solid state. Specifically, with respect to the total weight of RD and RQ9, the crystal is preferably 80 wt % or more, more preferably 90 wt % or more, still more preferably 95 wt % or more, and most preferably 98 wt % or more.

The content of RD and RQ9 (RD and RQ9 total weight/total composition weight) is not particularly limited, but is typically 0.001 wt % or more, preferably 0.005 wt % or more, more preferably 0.01 wt % or more, particularly preferably 0.015 wt % or more, and further preferably 0.02 wt % or more. The upper limit is not particularly limited, but is 10 wt % or less, preferably 8 wt % or less, more preferably 6 wt % or less, particularly preferably 4 wt % or less, and still more preferably 2 wt % or less. That is, the content of RD and RQ9 can typically be in the range of 0.001 to 10 wt %, preferably 0.005 to 8 wt %, more preferably 0.01 to 6 wt %, particularly preferably 0.015 to 4 wt %, and further preferably 0.02 to 2 wt %. The content of RD in the composition is within the range of preferably 0.0005 to 5 wt %, more preferably 0.001 to 3 wt %, and the content of RQ9 is within the range of preferably 0.002 to 4 wt %, more preferably 0.005 to 2 wt %.

The content of RD in the composition is, for example, 0.0005 wt % or more, in one embodiment, 0.001 wt % or more and, for example, 5 wt % or less, in one embodiment, 3 wt % or less, and, for example, within the range of 0.0005 wt % to 5 wt %, in one embodiment, 0.001 wt % to 3 wt %, and the content of RQ9 is, for example, 0.002 wt % or more, in one embodiment, 0.005 wt % or more, and, for example, 4 wt % or less, in one embodiment, 2 wt % or less, and, for example, within the range of 0.002 wt % to 4 wt %, in one embodiment, 0.005 wt % to 2 wt %.

And, in the reduced coenzyme Q10 composition according to the present embodiment, by adjusting a peak area ratio in the chromatogram of RD to RQ9 in the composition within a specific range, although the reason is not clear, it has become clear that the polymorphic transition of reduced coenzyme Q10 (QH) is suppressed. Specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more. The above peak area ratio (RD/RQ9) may be 0.5 or more, 0.8 or more, 1.0 or more, 1.5 or more, or 2.0 or more. The upper limit of the above peak area ratio (RD/RQ9) is not particularly limited, but may be 10.0 or less, 8.0 or less, 6.0 or less, 4.0 or less, or 3.0 or less. That is, the above peak area ratio may be 0.3 or more, and may be within the range of 0.3 to 10.0, 0.5 to 10.0, 0.5 to 3.0, 0.8 to 8.0, 1.0 to 6.0, 1.0 to 2.0, 1.5 to 4.0, or 2.0 to 3.0.

In another embodiment, specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more. The above peak area ratio (RD/RQ9) may be, for example, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, or in one embodiment, 2.0 or more. The upper limit of the above peak area ratio (RD/RQ9) is not particularly limited, but may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.0 or less, in one embodiment, 2.8 or less, in one embodiment, 2.6 or less, in one embodiment, 2.4 or less, in one embodiment, 2.2 or less, or in one embodiment, 2.1 or less. That is, the above peak area ratio may be 0.3 or more, and, for example, may be within the range of 0.3 to 10.0, in one embodiment, 0.3 to 9.0, in one embodiment, 0.3 to 8.0, in one embodiment, 0.3 to 7.0, in one embodiment, 0.3 to 6.0, in one embodiment, 0.3 to 5.0, in one embodiment, 0.3 to 4.0, in one embodiment, 0.3 to 3.0, in one embodiment, 0.3 to 2.8, in one embodiment, 0.3 to 2.6, in one embodiment, 0.3 to 2.4, in one embodiment, 0.3 to 2.2, in one embodiment, 0.3 to 2.1, in one embodiment, 0.4 to 2.1, in one embodiment, 0.5 to 2.1, in one embodiment, 0.6 to 2.1, in one embodiment, 0.7 to 2.1, in one embodiment, 0.8 to 2.1, in one embodiment, 0.9 to 2.1, in one embodiment, 1.0 to 2.1, in one embodiment, 1.1 to 2.1, in one embodiment, 1.2 to 2.1, in one embodiment, 1.3 to 2.1, in one embodiment, 1.4 to 2.1, in one embodiment, 1.5 to 2.1, in one embodiment, 1.6 to 2.1, in one embodiment, 1.7 to 2.1, in one embodiment, 1.8 to 2.1, in one embodiment, 1.9 to 2.1, in one embodiment, 2.0 to 2.1, in one embodiment, 0.5 to 10.0, in one embodiment, 0.5 to 3.0, in one embodiment, 0.8 to 8.0, in one embodiment, 1.0 to 6.0, in one embodiment, 1.0 to 2.0, in one embodiment, 1.5 to 4.0, or in one embodiment, 2.0 to 3.0.

Here, the peak area of each compound in the chromatogram can be calculated by an automatic integration method (in which an integrator automatically calculates from the signal intensity of the detector). In addition, the identification of each peak is performed by mass spectrometry. Furthermore, the peak area of each compound in the chromatogram can be appropriately changed by adjusting the content of each compound in the composition.

In the present embodiment, in the chromatogram obtained by high performance liquid chromatography under the above conditions, the area of the peak containing each of reduced coenzyme Q9 (RQ9), reduced demethoxy Q10 (RD), reduced coenzyme Q10 (QH), decaprenylphenol and reduced coenzyme Q11 (RQ11) to be described later, and oxidized coenzyme Q10 (QX) refers to the area of the peak detected at the retention time (RT) shown in the table below, or at the relative retention time (RRT) based on the retention time (RT, 14.72 min in Table 1) of the peak top of QH.

TABLE 1

| | Reduced | Reduced | Reduced |
|---|---|---|---|
| | Coenzyme Q9 | Demethoxy Q10 | Coenzyme Q10 |
| | (RQ9) | (RD) | (QH) |
| RT(min) | 10.10 to 10.75 | 12.90 to 13.90 | 14.00 to 15.75 |
| RRT | 0.69 to 0.73 | 0.88 to 0.94 | 0.95 to 1.07 |

| | | Reduced | Oxidized |
|---|---|---|---|
| | | Coenzyme Q11 | Coenzyme Q10 |
| | Decaprenylphenol | (RQ11) | (QX) |
| RT(min) | 19.75 to 20.7 | 21.30 to 22.0 | 24.75 to 26.25 |
| RRT | 1.34 to 1.41 | 1.45 to 1.49 | 1.68 to 1.78 |

In the present embodiment, in the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but, for example, may be 0.0005 or more, in one embodiment, 0.001 or more, in one embodiment, 0.002 or more, in one embodiment, 0.003 or more, in one embodiment, 0.004 or more, in one embodiment, 0.005 or more, and in one embodiment, 0.01 or less, in one embodiment, 0.008 or less, in one embodiment, 0.006 or less.

In the present embodiment, in the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but is within the range of preferably 0.0005 to 0.01, more preferably 0.001 to 0.008, still more preferably 0.002 to 0.008, and particularly preferably 0.004 to 0.006.

In the present embodiment, in the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but, for example, is within the range of 0.0005 to 0.01, in one embodiment, 0.001 to 0.008, in one embodiment, 0.001 to 0.006, in one embodiment, 0.002 to 0.006, in one embodiment, 0.003 to 0.006, in one embodiment, 0.004 to 0.006, in one embodiment, 0.005 to 0.006, and in one embodiment 0.002 to 0.008.

Further, the area ratio (RQ9/QH) of the peak containing reduced coenzyme Q9 (RQ9) to the peak containing reduced coenzyme Q10 (QH) is not particularly limited, but, for example, may be 0.001 or more, in one embodiment, may be 0.002 or more, in one embodiment, may be 0.01 or less, in one embodiment, may be 0.008 or less, in one embodiment, may be 0.007 or less, in one embodiment, may be 0.006 or less, in one embodiment, may be 0.005 or less, in one embodiment, may be 0.004 or less, and in one embodiment may be 0.003 or less.

In addition, the area ratio (RQ9/QH) of the peak containing reduced coenzyme Q9 (RQ9) to the peak containing reduced coenzyme Q10 (QH) is not particularly limited, but is within the range of preferably 0.001 to 0.01, more preferably 0.001 to 0.006, still more preferably 0.001 to 0.005, and particularly preferably 0.002 to 0.005.

Further, the area ratio (RQ9/QH) of the peak containing reduced coenzyme Q9 (RQ9) to the peak containing reduced coenzyme Q10 (QH) is not particularly limited, but, for example, is within the range of 0.001 to 0.01, in one embodiment, 0.001 to 0.008, in one embodiment, 0.001 to 0.007, in one embodiment, 0.001 to 0.006, in one embodiment, 0.001 to 0.005, in one embodiment, 0.001 to 0.004, in one embodiment, 0.001 to 0.003, in one embodiment, 0.002 to 0.003, in one embodiment, 0.002 to 0.005.

In the present embodiment, in the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but, for example, may be 0.001 or more, in one embodiment, may be 0.005 or more, in one embodiment, may be 0.007 or more, in one embodiment, may be 0.008 or more, in one embodiment, may be 0.009 or more, in one embodiment, may be 0.01 or more, and in one embodiment may be 0.03 or less, in one embodiment, may be 0.02 or less, in one embodiment, may be 0.015 or less, in one embodiment, may be 0.012 or less, and in one embodiment may be 0.01 or less.

In the present embodiment, in the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but is within the range of preferably 0.001 to 0.03, more preferably 0.005 to 0.02, still more preferably 0.007 to 0.015, and particularly preferably 0.007 to 0.01.

In the present embodiment, in the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but, for example, is within the range of 0.001 to 0.03, in one embodiment, 0.005 to 0.03, in one embodiment, 0.007 to 0.03, in one embodiment, 0.007 to 0.02, in one embodiment, 0.007 to 0.015, in one embodiment, 0.007 to 0.012, in one embodiment, 0.008 to 0.012, in one embodiment, 0.009 to 0.012, in one embodiment, 0.01 to 0.012, in one embodiment, 0.005 to 0.02, in one embodiment, 0.007 to 0.01, in one embodiment, 0.008 to 0.01, and in one embodiment, 0.009 to 0.01.

As a further effect, by adjusting the peak area ratio in the chromatogram of reduced demethoxy Q10 (RD) to RQ9 in the composition within a specific range, the in vivo absorbability of reduced coenzyme Q10 improves. The term “in vivo” means inside the body, inside a tissue, or inside a cell, of an organism. The organism is not particularly limited, but mammals, birds, fish, or the like are preferred. The above-described tissue is not particularly limited, and examples thereof include the skin, the digestive system, and the like. The digestive system is not particularly limited, and examples thereof include the digestive tract, the pancreas, the liver, the gallbladder, and the like. The digestive tract is not particularly limited, and examples thereof include the esophagus, stomach, duodenum, small intestine, and large intestine, and the like. “In vivo absorption” means that the reduced coenzyme Q10 that is the subject is taken up in vivo. In one embodiment, “in vivo absorption” means that the reduced coenzyme Q10 that is the subject is taken up in a state in which it has been decomposed by a digestive enzyme or the like. “Improvement of in vivo absorbability” means that the reduced coenzyme Q10 that is the subject becomes more easily absorbed in vivo, as compared with reduced coenzyme Q10 alone. The term “digestive fluid” means a generic term for liquids containing digestive enzymes secreted from digestive glands in the digestive system. The digestive fluid is not particularly limited, and examples thereof include saliva, gastric juice, bile, pancreatic juice, and intestinal juice and the like. “The improvement in digestive fluid solubility” means that the reduced coenzyme Q10 that is the subject becomes more readily subject to dissolution in digestive fluid, as compared with reduced coenzyme Q10 alone. Digestive fluid solubility can be evaluated, for example, by the solubility with respect to digestive fluid. In one embodiment, in vivo absorbability improves as digestive fluid solubility improves.

Furthermore, the reduced coenzyme Q10 composition of the present embodiment, in addition to the above RD and RQ9, may contain, as necessary, a QH analog. Examples of the QH analogs, including decaprenylphenol and reduced coenzyme Q11 (hereinafter may be referred to as “RQ11”) as shown below, can be given.

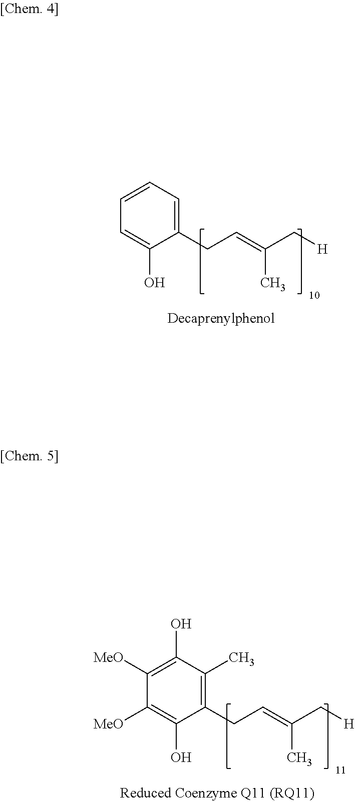

QH analogs such as the above decaprenylphenol and RQ11 can, similarly to QH, be obtained by known methods such as synthesis, fermentation, and extraction from natural products, as well as by combining those with a reduction reaction as needed. Preferably, QH analogs, such as decaprenylphenol and RQ11, that are generated together as by-products when reduced coenzyme Q10 is produced by reducing a composition containing existing oxidized coenzyme Q10 with a general reducing agent, for example, sodium dithionite, sodium borohydride, ascorbic acids, or the like, may be used as they are.

In a reduced coenzyme Q10 composition, the content of QH analog (RD, RQ11, RQ9 and decaprenylphenol) (total weight of QH analogs/total composition weight) is not particularly limited, but is typically 8 wt % or less, preferably 7 wt % or less, more preferably 6 wt % or less, particularly preferably 5 wt % or less, still more preferably 4 wt % or less, and is typically 0.006 wt % or more, preferably 0.008 wt % or more, more preferably 0.01 wt % or more. The total content of QH analogs (RD, RQ11, RQ9, and decaprenylphenol) in the composition is typically 0.006 to 8 wt %, preferably 0.006 to 7 wt %, more preferably 0.008 to 6 wt %, still more preferably 0.008 to 5 wt %, and particularly preferably 0.01 to 4 wt %.

The content of decaprenylphenol (decaprenylphenol weight/total composition weight) in the reduced coenzyme Q10 composition is, in one embodiment, 0.001 wt % or more, in one embodiment, 0.002 wt % or more, in one embodiment, 3 wt % or less, in one embodiment, 2 wt % or less, and the content of RQ11 is, in one embodiment, 0.005 wt % or more, in one embodiment, 0.01 wt % or more, in one embodiment, 5 wt % or less, and in one embodiment, 3 wt % or less.

The content of decaprenylphenol (weight of decaprenylphenol/total composition weight) in the reduced coenzyme Q10 composition is within the range of preferably 0.001 to 3 wt %, more preferably 0.002 to 2 wt %, and the content of RQ11 is within the range of preferably 0.005 to 5 wt %, more preferably 0.01 to 3 wt %.

In the present embodiment, in the chromatogram obtained by high performance liquid chromatography under the above conditions, the area ratio (decaprenylphenol/QH) of the peak containing decaprenylphenol to the peak containing QH is not particularly limited, but, for example, may be 0.001 or more, in one embodiment, 0.002 or more, in one embodiment, 0.003 or more, in one embodiment, 0.004 or more, in one embodiment, 0.005 or more, in one embodiment, 0.006 or more, in one embodiment, 0.007 or more, in one embodiment, 0.008 or more, in one embodiment, 0.009 or more, in one embodiment, 0.05 or less, in one embodiment, 0.03 or less, and in one embodiment, 0.01 or less.

In the present embodiment, in the chromatogram obtained by high performance liquid chromatography under the above conditions, the area ratio (decaprenylphenol/QH) of the peak containing decaprenylphenol to the peak containing QH is not particularly limited, but is within the range of preferably 0.001 to 0.05, more preferably 0.003 to 0.03, and still more preferably 0.005 to 0.01.

In the present embodiment, in the chromatogram obtained by high performance liquid chromatography under the above conditions, the area ratio (decaprenylphenol/QH) of the peak containing decaprenylphenol to the peak containing QH is not particularly limited, but is, for example, within the range of 0.001 to 0.05, in one embodiment, 0.001 to 0.03, in one embodiment, 0.001 to 0.01, in one embodiment, 0.002 to 0.01, in one embodiment, 0.003 to 0.01, in one embodiment, 0.004 to 0.01, in one embodiment, 0.005 to 0.01, in one embodiment, 0.006 to 0.01, in one embodiment, 0.007 to 0.01, in one embodiment, 0.008 to 0.01, in one embodiment, 0.009 to 0.01, and in one embodiment, 0.003 to 0.03.

In the chromatogram obtained by high performance liquid chromatography under the above conditions, the area ratio (RQ11/QH) of the peak containing RQ11 to the peak containing QH is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.01 or more, in one embodiment, 0.015 or more, in one embodiment, 0.016 or more, in one embodiment, 0.017 or more, in one embodiment, 0.018 or more, in one embodiment, 0.019 or more, in one embodiment, 0.02 or more, in one embodiment, 0.05 or less, in one embodiment, 0.04 or less, in one embodiment, 0.03 or less, in one embodiment, 0.025 or less, and in one embodiment, 0.02 or less.

In the chromatogram obtained by high performance liquid chromatography under the above conditions, the area ratio (RQ11/QH) of the peak containing RQ11 to the peak containing QH is not particularly limited, but is within the range of preferably 0.001 to 0.05, more preferably 0.005 to 0.04, and even more preferably 0.01 to 0.03.

In the chromatogram obtained by high performance liquid chromatography under the above conditions, the area ratio (RQ11/QH) of the peak containing RQ11 to the peak containing QH is not particularly limited, but, for example, is within the range of 0.001 to 0.05, in one embodiment, 0.005 to 0.05, in one embodiment, 0.006 to 0.05, in one embodiment, 0.006 to 0.04, in one embodiment, 0.006 to 0.03, in one embodiment, 0.006 to 0.025, in one embodiment, 0.007 to 0.025, in one embodiment, 0.008 to 0.025, in one embodiment, 0.009 to 0.025, in one embodiment, 0.01 to 0.025, in one embodiment, 0.015 to 0.025, in one embodiment, 0.016 to 0.025, in one embodiment, 0.017 to 0.025, in one embodiment, 0.018 to 0.025, in one embodiment, 0.019 to 0.025, in one embodiment, 0.02 to 0.025, in one embodiment, 0.016 to 0.02, in one embodiment, 0.005 to 0.04, and in one embodiment 0.01 to 0.03.

In the chromatogram obtained by high performance liquid chromatography under the above conditions, the area ratio (decaprenylphenol/RQ9) of the peak containing decaprenylphenol to the peak containing RQ9 is not particularly limited, but may be, for example, 0.1 or more, in one embodiment, 0.2 or more, in one embodiment, 0.3 or more, in one embodiment, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.2 or more, in one embodiment, 1.4 or more, in one embodiment, 1.6 or more, in one embodiment, 1.8 or more, in one embodiment, 2.0 or more, in one embodiment, 2.2 or more, in one embodiment, 2.3 or more, in one embodiment, 2.4 or more, in one embodiment, 2.6 or more, in one embodiment, 2.8 or more, in one embodiment, 3.0 or more, in one embodiment, 3.2 or more, in one embodiment, 3.4 or more, or in one embodiment, 3.6 or more, and the upper limit may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.8 or less, or in one embodiment, 3.7 or less.

In the chromatogram obtained by high performance liquid chromatography under the above conditions, the area ratio (decaprenylphenol/RQ9) of the peak containing decaprenylphenol to the peak containing RQ9 is not particularly limited, but may be within the range of 0.1 to 10.0, 0.1 to 8.0, 0.1 to 6.0, 0.1 to 5.0, 0.1 to 4.0, 0.5 to 4.0, or 1.0 to 4.0.

In the chromatogram obtained by high performance liquid chromatography under the above conditions, the area ratio (decaprenylphenol/RQ9) of the peak containing decaprenylphenol to the peak containing RQ9 is not particularly limited, but may be, for example, within the range of 0.1 to 10.0, in one embodiment, 0.1 to 9.0, in one embodiment, 0.1 to 8.0, in one embodiment, 0.1 to 7.0, in one embodiment, 0.1 to 6.0, in one embodiment, 0.1 to 5.0, in one embodiment, 0.1 to 4.0, in one embodiment, 0.2 to 4.0, in one embodiment, 0.3 to 4.0, in one embodiment, 0.4 to 4.0, in one embodiment, 0.5 to 4.0, in one embodiment, 0.6 to 4.0, in one embodiment, 0.7 to 4.0, in one embodiment, 0.8 to 4.0, in one embodiment, 0.9 to 4.0, in one embodiment, 1.0 to 4.0, in one embodiment, 1.2 to 4.0, in one embodiment, 1.4 to 4.0, in one embodiment, 1.6 to 4.0, in one embodiment, 1.8 to 4.0, in one embodiment, 2.0 to 4.0, in one embodiment, 2.2 to 4.0, in one embodiment, 2.3 to 4.0, in one embodiment, 2.4 to 4.0, in one embodiment, 2.6 to 4.0, in one embodiment, 2.8 to 4.0, in one embodiment, 3.0 to 4.0, in one embodiment, 3.2 to 4.0, in one embodiment, 3.4 to 4.0, in one embodiment, 3.6 to 4.0, in one embodiment, 3.6 to 3.8, or in one embodiment, 3.6 to 3.7.

In a chromatogram obtained by high performance liquid chromatography under the above conditions, the area ratio (RQ11/RQ9) of a peak containing RQ11 to a peak containing RQ9 is not particularly limited, but may be, for example, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, in one embodiment, 2.0 or more, in one embodiment, 2.2 or more, in one embodiment, 2.4 or more, in one embodiment, 2.6 or more, in one embodiment, 2.8 or more, in one embodiment, 3.0 or more, in one embodiment, 3.2 or more, in one embodiment, 3.3 or more, in one embodiment, 3.4 or more, in one embodiment, 3.6 or more, in one embodiment, 3.8 or more, in one embodiment, 4.0 or more, in one embodiment, 4.2 or more, in one embodiment, 4.4 or more, in one embodiment, 4.6 or more, in one embodiment, 4.8 or more, in one embodiment, 5.0 or more, in one embodiment, 5.5 or more, in one embodiment, 6.0 or more, in one embodiment, 6.5 or more, in one embodiment, 7.0 or more, in one embodiment, 7.5 or more, or in one embodiment 8.0 or more, and may be, for example, 15.0 or less, in one embodiment, 12.0 or less, in one embodiment, 10.0 or less, in one embodiment, 9.5 or less, in one embodiment, 9.0 or less, in one embodiment, 8.5 or less, in one embodiment, 8.4 or less, in one embodiment, 8.3 or less, in one embodiment, 8.2 or less, or in one embodiment, 8.1 or less.

In the chromatogram obtained by high performance liquid chromatography under the above conditions, the area ratio (RQ11/RQ9) of the peak containing RQ11 to the peak containing RQ9 is not particularly limited, but may be within the range of 1.0 to 15.0, 1.2 to 15.0, 1.4 to 12.0, 1.5 to 10.0, 1.8 to 9.5, or 2.0 to 9.0.

In the chromatogram obtained by high performance liquid chromatography under the above conditions, the area ratio (RQ11/RQ9) of the peak containing RQ11 to the peak containing RQ9 is not particularly limited, but, for example, may be within the range of 1.0 to 15.0, in one embodiment, 1.0 to 12.0, in one embodiment, 1.0 to 10.0, in one embodiment, 1.0 to 9.5, in one embodiment, 1.0 to 9.0, in one embodiment, 1.0 to 8.5, in one embodiment, 1.0 to 8.4, in one embodiment, 1.0 to 8.3, in one embodiment, 1.0 to 8.2, in one embodiment, 1.0 to 8.1, in one embodiment, 1.1 to 8.1, in one embodiment, 1.2 to 8.1, in one embodiment, 1.3 to 8.1, in one embodiment, 1.4 to 8.1, in one embodiment, 1.5 to 8.1, in one embodiment, 1.6 to 8.1, in one embodiment, 1.7 to 8.1, in one embodiment, 1.8 to 8.1, in one embodiment, 1.9 to 8.1, in one embodiment, 2.0 to 8.1, in one embodiment, 2.2 to 8.1, in one embodiment, 2.4 to 8.1, in one embodiment, 2.6 to 8.1, in one embodiment, 2.8 to 8.1, in one embodiment, 3.0 to 8.1, in one embodiment, 3.2 to 8.1, in one embodiment, 3.3 to 8.1, in one embodiment, 3.4 to 8.1, in one embodiment, 3.6 to 8.1, in one embodiment, 3.8 to 8.1, in one embodiment, 4.0 to 8.1, in one embodiment, 4.2 to 8.1, in one embodiment, 4.4 to 8.1, in one embodiment, 4.6 to 8.1, in one embodiment, 4.8 to 8.1, in one embodiment, 5.0 to 8.1, in one embodiment, 5.5 to 8.1, in one embodiment, 6.0 to 8.1, in one embodiment, 6.5 to 8.1, in one embodiment, 7.0 to 8.1, in one embodiment, 7.5 to 8.1, in one embodiment, 8.0 to 8.1, in one embodiment, 1.2 to 15.0, in one embodiment, 1.4 to 12.0, in one embodiment, 1.5 to 10.0, in one embodiment, 1.8 to 9.5, or in one embodiment 2.0 to 9.0.

In the chromatogram obtained by high performance liquid chromatography under the above conditions, the area ratio (decaprenylphenol/RD) of the peak containing decaprenylphenol to the peak containing RD is not particularly limited and may be, for example, 0.1 or more, in one embodiment, 0.5 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, and may be, in one embodiment, 10 or less, in one embodiment, 5.0 or less, in one embodiment, 3.0 or less, in one embodiment, 2.5 or less, and in one embodiment, 2.0 or less.

In the chromatogram obtained by high performance liquid chromatography under the above conditions, the area ratio (decaprenylphenol/RD) of the peak containing decaprenylphenol to the peak containing RD is not particularly limited, but is within the range of preferably 0.1 to 10, more preferably 0.5 to 5.0, and still more preferably 1.0 to 3.0.

In the chromatogram obtained by high performance liquid chromatography under the above conditions, the area ratio (decaprenylphenol/RD) of the peak containing decaprenylphenol to the peak containing RD is not particularly limited, but, for example, is within the range of 0.1 to 10, in one embodiment, 0.5 to 10, in one embodiment, 0.7 to 10, in one embodiment, 0.7 to 5.0, in one embodiment, 0.7 to 3.0, in one embodiment, 0.7 to 2.5, in one embodiment, 0.7 to 2.0, in one embodiment, 0.8 to 2.0, in one embodiment, 0.9 to 2.0, in one embodiment, 1.0 to 2.0, in one embodiment, 1.1 to 2.0, in one embodiment, 1.2 to 2.0, in one embodiment, 1.3 to 2.0, in one embodiment, 1.4 to 2.0, in one embodiment, 1.5 to 2.0, in one embodiment, 1.6 to 2.0, in one embodiment, 1.7 to 2.0, in one embodiment, 1.8 to 2.0, in one embodiment, 0.5 to 5.0, and in one embodiment, 1.0 to 3.0.

In the chromatogram obtained by high performance liquid chromatography under the above conditions, the area ratio (RD/RQ11) of the peak containing RD to the peak containing RQ11 is not particularly limited, but, for example, it is 0.1 or more, in one embodiment, 0.2 or more, specifically, 0.19 or more, in one embodiment, 0.20 or more, in one embodiment, 0.21 or more, and in one embodiment, 1.0 or less, in one embodiment, 0.8 or less, in one embodiment, 0.5 or less, specifically, 0.50 or less, in one embodiment, 0.49 or less, in one embodiment, 0.48 or less, and in one embodiment, 0.47 or less.

In the chromatogram obtained by high performance liquid chromatography under the above conditions, the area ratio (RD/RQ11) of the peak containing RD to the peak containing RQ11 is not particularly limited, but is within the range of preferably 0.1 to 1.0, more preferably 0.2 to 0.8, and still more preferably 0.2 to 0.5.

In the chromatogram obtained by high performance liquid chromatography under the above conditions, the area ratio (RD/RQ11) of the peak containing RD to the peak containing RQ11 is not particularly limited, but is, for example, within the range of 0.1 to 1.0, in one embodiment, 0.2 to 0.8, in one embodiment, 0.2 to 0.5, specifically, 0.19 to 0.50, in one embodiment, 0.20 to 0.49, in one embodiment, 0.21 to 0.48, and in one embodiment, 0.21 to 0.47.

In the chromatogram obtained by high performance liquid chromatography under the above conditions, the area ratio (decaprenylphenol/RQ11) of the peak containing decaprenylphenol to the peak containing RQ11 is not particularly limited, but may be, for example, 0.01 or more, in one embodiment, 0.03 or more, in one embodiment, 0.05 or more, in one embodiment, 0.07 or more, in one embodiment, 0.08 or more, in one embodiment, 0.1 or more, in one embodiment, 0.2 or more, in one embodiment, 0.25 or more, in one embodiment, 0.3 or more, in one embodiment, 0.35 or more, in one embodiment, 0.4 or more, or in one embodiment, 0.45 or more, and may be, for example, 1.5 or less, in one embodiment, 1.1 or less, in one embodiment, 1.0 or less, in one embodiment 0.8 or less, in one embodiment, 0.7 or less, in one embodiment, 0.6 or less, or in one embodiment 0.5 or less.

In the chromatogram obtained by high performance liquid chromatography under the above conditions, the area ratio (decaprenylphenol/RQ11) of the peak containing decaprenylphenol to the peak containing RQ11 is not particularly limited, but may be within the range of 0.01 to 1.5, 0.03 to 1.0, 0.05 to 0.8, 0.07 to 0.7, 0.08 to 0.6, or 0.1 to 0.5.

In the chromatogram obtained by high performance liquid chromatography under the above conditions, the area ratio (decaprenylphenol/RQ11) of the peak containing decaprenylphenol to the peak containing RQ11 is not particularly limited, but may be, for example, within the range of 0.01 to 1.5, in one embodiment, 0.03 to 1.5, in one embodiment, 0.05 to 1.5, in one embodiment, 0.07 to 1.5, in one embodiment, 0.08 to 1.5, in one embodiment, 0.1 to 1.5, in one embodiment, 0.2 to 1.5, in one embodiment, 0.2 to 1.1, in one embodiment, 0.2 to 1.0, in one embodiment, 0.2 to 0.8, in one embodiment, 0.2 to 0.7, in one embodiment, 0.2 to 0.6, in one embodiment, 0.2 to 0.5, in one embodiment, 0.25 to 0.5, in one embodiment, 0.3 to 0.5, in one embodiment, 0.35 to 0.5, in one embodiment, 0.4 to 0.5, in one embodiment, 0.45 to 0.5, in one embodiment, 0.03 to 1.0, in one embodiment, 0.05 to 0.8, in one embodiment, 0.07 to 0.7, in one embodiment, 0.08 to 0.6, or in one embodiment 0.1 to 0.5.

In the chromatogram obtained by high performance liquid chromatography under the above conditions, the ratio ((decaprenylphenol+RQ9)/QH) of the total area of the peak containing decaprenylphenol and the peak containing reduced coenzyme Q9 (RQ9) to the area of the peak containing reduced coenzyme Q10 (QH) is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.025 or more, in one embodiment, 0.030 or more, in one embodiment, 0.10 or less, in one embodiment, 0.050 or less, in one embodiment, 0.040 or less, in one embodiment, 0.030 or less, in one embodiment, 0.020 or less, in one embodiment, 0.025 or less, in one embodiment, 0.020 or less, and in one embodiment, 0.015 or less.

In the chromatogram obtained by high performance liquid chromatography under the above conditions, the ratio ((decaprenylphenol+RQ9)/QH) of the total area of the peak containing decaprenylphenol and the peak containing reduced coenzyme Q9 (RQ9) to the area of the peak containing reduced coenzyme Q10 (QH) is not particularly limited, but is, for example, within the range of 0.001 to 0.10, in one embodiment, 0.005 to 0.050, in one embodiment, 0.007 to 0.040, in one embodiment, 0.008 to 0.030, in one embodiment, 0.009 to 0.025, in one embodiment, 0.010 to 0.020, and in one embodiment 0.010 to 0.015.

In the chromatogram obtained by high performance liquid chromatography under the above conditions, the ratio ((decaprenylphenol+RQ11)/QH) of the total area of the peak containing decaprenylphenol and the peak containing reduced coenzyme Q11 (RQ11) to the area of the peak containing reduced coenzyme Q10 (QH) is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.025 or more, in one embodiment, 0.030 or more, in one embodiment, 0.10 or less, in one embodiment, 0.050 or less, in one embodiment, 0.040 or less, in one embodiment, 0.035 or less, in one embodiment, 0.032 or less, and in one embodiment, 0.031 or less.

In the chromatogram obtained by high performance liquid chromatography under the above conditions, the ratio ((decaprenylphenol+RQ11)/QH) of the total area of the peak containing decaprenylphenol and the peak containing reduced coenzyme Q11 (RQ11) to the peak containing reduced coenzyme Q10 (QH) is not particularly limited but is, for example, within the range of 0.001 to 0.10, in one embodiment, 0.005 to 0.050, in one embodiment, 0.007 to 0.040, in one embodiment, 0.008 to 0.035, in one embodiment, 0.009 to 0.032, in one embodiment, 0.010 to 0.031, in one embodiment, 0.012 to 0.031, in one embodiment, 0.013 to 0.031, in one embodiment, 0.014 to 0.031, in one embodiment, 0.015 to 0.031, in one embodiment, 0.020 to 0.031, and in one embodiment, 0.025 to 0.031.

In the chromatogram obtained by high performance liquid chromatography under the above conditions, the ratio ((RQ9+RQ11)/QH) of the total area of the peaks containing reduced coenzyme Q9 (RQ9) and the peaks containing reduced coenzyme Q11 (RQ11) to the area of the peak containing reduced coenzyme Q10 (QH) is not particularly limited, but, for example, may be 0.001 or more, in one embodiment, 0.005 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.021 or more, in one embodiment, 0.022 or more, and may be, in one embodiment, 0.10 or less, in one embodiment, 0.050 or less, in one embodiment, 0.040 or less, in one embodiment, 0.035 or less, in one embodiment, 0.030 or less, in one embodiment, 0.029 or less, in one embodiment, 0.028 or less, in one embodiment, 0.027 or less, in one embodiment, 0.026 or less, and in one embodiment 0.025 or less.

In the chromatogram obtained by high performance liquid chromatography under the above conditions, the ratio ((RQ9+RQ11)/QH) of the total area of the peak containing reduced coenzyme Q9 (RQ9) and the peak containing reduced coenzyme Q11 (RQ11) to the peak containing reduced coenzyme Q10 (QH) is not particularly limited, but is, for example, within the range of 0.001 to 0.10, in one embodiment, 0.005 to 0.050, in one embodiment, 0.010 to 0.040, in one embodiment, 0.011 to 0.035, in one embodiment, 0.012 to 0.030, in one embodiment, 0.013 to 0.029, in one embodiment, 0.014 to 0.028, in one embodiment, 0.014 to 0.027, in one embodiment, 0.015 to 0.026, in one embodiment, 0.016 to 0.026, in one embodiment, 0.017 to 0.026, in one embodiment, 0.018 to 0.026, in one embodiment, 0.020 to 0.026, and in one embodiment, 0.022 to 0.026.

In the present embodiment, in a chromatogram obtained by high performance liquid chromatography under the above conditions, the area ratio (total value of the peak areas of the QH analogs/peak area of QH) of peaks containing QH analogs (RD, RQ11, RQ9, and decaprenylphenol) to a peak containing QH is not particularly limited, but, for example, may be 0.001 or more, in one embodiment, 0.006 or more, in one embodiment, 0.007 or more, in one embodiment, 0.012 or more, in one embodiment, 0.02 or more, in one embodiment, 0.022 or more, in one embodiment, 0.024 or more, in one embodiment, 0.03 or more, in one embodiment, 0.033 or more, in one embodiment, 0.035 or more, in one embodiment, 0.038 or more, in one embodiment, 0.10 or less, in one embodiment, 0.08 or less, in one embodiment, 0.06 or less, in one embodiment, 0.05 or less, and in one embodiment 0.04 or less.

In the present embodiment, in the chromatogram obtained by high performance liquid chromatography under the above conditions, the area ratio (total value of the peak areas pf QH analogs/peak area of QH) of the peaks containing QH analogs (RD, RQ11, RQ9, and decaprenylphenol) to the peak containing QH is not particularly limited, but is within the range of preferably 0.001 to 0.10, more preferably 0.006 to 0.08, still more preferably 0.007 to 0.06, particularly preferably 0.012 to 0.05, and most preferably 0.02 to 0.04.

In the present embodiment, in the chromatogram obtained by high performance liquid chromatography under the above conditions, the area ratio (total value of the peak areas of QH analogs/peak area of QH) of the peaks containing QH analogs (RD, RQ11, RQ9, and decaprenylphenol) to the peak containing QH is not particularly limited, but is, for example, within the range of 0.001 to 0.10, in one embodiment, 0.001 to 0.08, in one embodiment, 0.001 to 0.06, in one embodiment, 0.001 to 0.05, in one embodiment, 0.001 to 0.04, in one embodiment, 0.006 to 0.04, in one embodiment, 0.007 to 0.04, in one embodiment, 0.012 to 0.04, in one embodiment, 0.02 to 0.04, in one embodiment, 0.022 to 0.04, in one embodiment, 0.024 to 0.04, in one embodiment, 0.03 to 0.04, in one embodiment, 0.033 to 0.04, in one embodiment, 0.035 to 0.04, in one embodiment, 0.038 to 0.04, in one embodiment, 0.006 to 0.08, in one embodiment, 0.007 to 0.06, and in one embodiment 0.012 to 0.05.

The substances other than reduced coenzyme Q10 and its analogs, reduced demethoxy Q10, and reduced coenzyme Q9 contained in the composition of the present embodiment are not particularly limited, and examples thereof include an excipient, a disintegrant, a lubricant, a binder, an emulsifier, a colorant, an anti-caking agent, an absorption enhancer, a dissolution aid, a stabilizer, a flavoring agent, fats and oils, a surfactant, a higher fatty acid, ethanol, water, an active ingredient other than reduced coenzyme Q10, an antioxidant, and the like.

Further, the form of the composition according to the present embodiment is not particularly limited, and can be appropriately selected as necessary. Specifically, examples thereof include oral preparations such as tablets, powders, chewable tablets, pills, capsules, granules, fine granules, sustained-release preparations, suspensions, emulsion preparations, syrups, elixirs, and the like, and non-oral preparations such as injections, suppositories, topical preparations, patch preparations, and the like. Since reduced coenzyme Q10 has excellent oral absorbability, the form of an oral preparation is particularly preferred. Furthermore, the reduced coenzyme Q10 composition according to the present embodiment can also be in the form of a capsule, for example, a capsule obtained by dissolution of reduced coenzyme Q10 in an oil component and sealing it in a capsule. Among these, a coenzyme Q10-containing composition in solid form is preferred. Hereinafter, the case where it is used as a composition in solid form, such as granules or tablets, will be described.

The form of a reduced coenzyme Q10 composition in solid form is not particularly limited, and examples thereof include forms such as powder, granules, tablet, hard capsule, and the like.

The type of emulsifier to be added to a reduced coenzyme Q10 composition in solid form is not particularly limited, but the use of an emulsifier that is acceptable for food, cosmetic, and/or pharmaceutical use is particularly preferred. For example, the use of an emulsifier having an HLB of 1 or more and 17 or less, preferably 2 or more and 16 or less, is possible. The use of two or more emulsifiers in combination is allowed. The preferably used specific examples of emulsifiers include one or two or more emulsifiers selected from the group consisting of glycerin fatty acid ester, sucrose fatty acid ester, retinol fatty acid ester, propylene glycol fatty acid ester, sorbitan fatty acid ester, and polysorbate.

From the viewpoint of suppressing the oxidation of reduced coenzyme Q10, among the above emulsifiers, emulsifiers that are neither in powder nor flake form are preferably used. Examples of the emulsifiers that are neither in powder nor flake form include emulsifiers that are in a liquid-to-sol form, a gel form, or a soft solid, and emulsifiers that are liquid, viscid liquid, viscous liquid, paste, pellets, waxy mass, wax, soft solid, or semi-solid are more preferred. As the emulsifier, an emulsifier having the above physical properties at 50° C. is preferred, and an emulsifier having the above physical properties at 25° C. is most preferred.

Examples of the emulsifiers that are neither in powder nor in flake form include emulsifiers in which the melting onset point in a measurement by a differential scanning calorimeter (DSC) with the heating rate set to 1° C./min or more and 20° C./min or less is 50° C. or lower, preferably 40° C. or lower, more preferably 25° C. or lower.

From another viewpoint, examples of the emulsifiers include emulsifiers in which the viscosity, in a measurement with the use of a B-type viscometer at a rotational speed of 10 rpm and a sample temperature of 50° C., is, for example, 150,000 mPa·s or less, preferably 100,000 mPa·s or less, more preferably 50,000 mPa·s or less, more preferably 30,000 mPa·s or less, and most preferably 25,000 mPa·s or less. The lower limit of the viscosity is not particularly limited as long as the viscosity is greater than 0 mPa·s, the viscosity is more preferably 1 mPa·s or more, particularly preferably 5 mPa·s or more, and most preferably 10 mPa·s or more. That is, the above viscosity can be, for example, within the range of 1 to 150,000 mPa·s, preferably within the range of 5 to 30,000 mPa·s, and particularly preferably within the range of 10 to 25,000 mPa·s.

The specific examples of the emulsifier include one or more selected from an ester compound of a polyol selected from monoglycerin, polyglycerin, sorbitan, polyoxyethylene sorbitan, sucrose, propylene glycol, polypropylene glycol, ethylene glycol, and polyethylene glycol, and a fatty acid which may have a substituent, and lecithin.

In polyglycerin, the number of units of glycerin may be 2 or more, and is preferably 2 or more and 10 or less. For example, diglycerin, triglycerin, tetraglycerin, pentaglycerin, hexaglycerin, and decaglycerin can be exemplified.

In polyoxyethylene sorbitan, the number of units of oxyethylene may be 2 or more, preferably 10 or more and 30 or less, more preferably 15 or more and 25 or less.

In polypropylene glycol, the number of units of propylene glycol may be 2 or more, and is preferably 2 or more and 10 or less.

In polyethylene glycol, the number of units of ethylene glycol may be 2 or more, and preferably is 2 or more and 10 or less.

Examples of the fatty acids that may have a substituent include straight-chain or branched-chain monocarboxylic or dicarboxylic fatty acids having 4 or more and 24 or less carbon atoms. As substituents, a hydroxy group and an acetoxy group can be exemplified. It is preferred that the number of substituents is 2 or less. Examples of the fatty acids that may have a substituent include lauric acid, oleic acid, caprylic acid, stearic acid, behenic acid, ricinoleic acid, succinic acid, and diacetyl tartaric acid.

In the ester compound of the polyol and the fatty acid, the number of bonds of the fatty acid to one molecule of the polyol is not particularly limited, and can be appropriately adjusted according to the HLB of the emulsifier for the intended purpose.

From the viewpoint of suppressing oxidation of reduced coenzyme Q10, the number of bonds of the fatty acid to one molecule of the polyol can be, for example, 12 or less, preferably 10 or less, more preferably 7 or less, more preferably 6 or less, more preferably 5 or less, more preferably 3 or less, more preferably 2 or less, and more preferably, is 1.

Specific examples of the ester compound of the polyol and the fatty acid include diglyceryl monooleate, monoglyceryl monocaprylate, diglyceryl monocaprylate, decaglyceryl pentaoleate, tetraglyceryl pentaoleate, pentaglyceryl trioleate, decaglyceryl monolaurate, hexaglyceryl monocaprylate, hexaglyceryl monooleate, pentaglyceryl monostearate, tetraglyceryl tristearate, decaglyceryl monobehenate, monostearate of mono- and diglycerides, monoglyceryl monooleate, glyceryl monostearate succinate, monoglyceryl succinate, glyceryl monostearate diacetyl tartrate, propylene glycol monooleate, sorbitan monooleate, sorbitan monostearate, sorbitan tristearate, monoglyceryl monolaurate, diglyceryl monolaurate, diglyceryl monomyristate, tetraglyceryl pentastearate, polyoxyethylene sorbitan monooleate, polyoxyethylene sorbitan monolaurate, polyoxyethylene sorbitan monostearate, condensed ricinoleic acid pentaglyceride, sucrose stearate, sucrose erucate, and sucrose oleate.

From the viewpoint of suppressing oxidation of reduced coenzyme Q10, the fatty acid in the ester compound of the polyol and the fatty acid is more preferably an unsaturated fatty acid. Examples of the unsaturated fatty acid include, for example, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, alpha-linolenic acid, gamma-linolenic acid, pinolenic acid, alpha-eleostearic acid, beta-eleostearic acid, Mead acid, dihomo-gamma-linolenic acid, eicosatrienoic acid, stearidonic acid, arachidonic acid, eicosatetraenoic acid, adrenic acid, boseopentaenoic acid, eicosapentaenoic acid, Osbond acid, clupanodonic acid, tetracosapentaenoic acid, docosahexaenoic acid, cetoleic acid, ricinoleic acid, and condensed ricinoleic acid, and oleic acid, condensed ricinoleic acid, linoleic acid, and erucic acid are more preferred, and oleic acid is particularly preferred.

Specific examples of the ester compounds of the polyol and the unsaturated fatty acid include monoglyceryl monooleate, monoglyceryl mono- and dioleate, monoglyceryl dioleate, mono- and diglyceryl monooleate, diglyceryl monooleate, diglyceryl mono- and dioleate, diglyceryl dioleate, diglyceryl trioleate, triglyceryl monooleate, triglyceryl dioleate, triglyceryl trioleate, triglyceryl tetraoleate, tetraglyceryl monooleate, tetraglyceryl dioleate, tetraglyceryl trioleate, tetraglyceryl tetraoleate, tetraglyceryl pentaoleate, pentaglyceryl monooleate, pentaglyceryl dioleate, pentaglyceryl trioleate, pentaglyceryl tetraoleate, pentaglyceryl pentaoleate, pentaglyceryl hexaoleate, hexaglyceryl monooleate, hexaglyceryl dioleate, hexaglyceryl trioleate, hexaglyceryl tetraoleate, hexaglyceryl pentaoleate, hexaglyceryl hexaoleate, hexaglyceryl heptaoleate, decaglyceryl monooleate, decaglyceryl dioleate, decaglyceryl trioleate, decaglyceryl tetraoleate, decaglyceryl pentaoleate, decaglyceryl hexaoleate, decaglyceryl heptaoleate, decaglyceryl octaoleate, decaglyceryl nonaoleate, decaglyceryl decaoleate, decaglyceryl dodecaoleate, diacetylglycerin monooleate, glyceryl monooleate lactate, glyceryl monooleate succinate, glyceryl monooleate citrate, glyceryl monooleate diacetyl tartrate, sucrose oleate, propylene glycol monooleate, sorbitan monooleate, sorbitan dioleate, sorbitan trioleate, polyoxyethylene sorbitan monooleate, phosphatidylcholine monopalmitate monooleate, phosphatidylcholine dilinoleate, monoglyceryl monoerucate, monoglyceryl mono- and dierucate, monoglyceryl dierucate, mono- and diglyceryl monoerucate, diglyceryl monoerucate, diglyceryl mono- and dierucate, diglyceryl dierucate, diglyceryl trierucate, triglyceryl monoerucate, triglyceryl dierucate, triglyceryl trierucate, triglyceryl tetraerucate, tetraglyceryl monoerucate, tetraglyceryl dierucate, tetraglyceryl trierucate, tetraglyceryl tetraerucate, tetraglyceryl pentaerucate, pentaglyceryl monoerucate, pentaglyceryl dierucate, pentaglyceryl trierucate, pentaglyceryl tetraerucate, pentaglyceryl pentaerucate, pentaglyceryl hexaerucate, hexaglyceryl monoerucate, hexaglyceryl dierucate, hexaglyceryl trierucate, hexaglyceryl tetraerucate, hexaglyceryl pentaerucate, hexaglyceryl hexaerucate, hexaglyceryl heptaerucate, decaglyceryl monoerucate, decaglyceryl dierucate, decaglyceryl trierucate, decaglyceryl tetraerucate, decaglyceryl pentaerucate, decaglyceryl hexaerucate, decaglyceryl heptaerucate, decaglyceryl octaerucate, decaglyceryl nonaerucate, decaglyceryl decaerucate, decaglyceryl dodecaerucate, diacetylglycerin monoerucate, glyceryl monoerucate lactate, glyceryl monoerucate succinate, glyceryl monoerucate citrate, glyceryl monoerucate diacetyl tartrate, sucrose erucate, propylene glycol monoerucate, sorbitan monoerucate, sorbitan dierucate, sorbitan trierucate, condensed ricinoleic acid monoglyceride, condensed ricinoleic acid diglyceride, condensed ricinoleic acid triglyceride, condensed ricinoleic acid tetraglyceride, condensed ricinoleic acid pentaglyceride, condensed ricinoleic acid hexaglyceride, condensed ricinoleic acid heptaglyceride, and condensed ricinoleic acid decaglyceride.

From the viewpoint of suppression of oxidation of reduced coenzyme Q10, as the ester compound of the polyol and the fatty acid, a polyoxyethylene sorbitan fatty acid ester is also preferably used. Specific examples of the polyoxyethylene sorbitan fatty acid ester include polyoxyethylene sorbitan monooleate, polyoxyethylene sorbitan monolaurate, and polyoxyethylene sorbitan monostearate.

The reduced coenzyme Q10 composition according to the present embodiment preferably contains an antioxidant in order to ensure oxidative stability, particularly in a solid dosage form. Here, the type of the antioxidant is not particularly limited. The use of two or more kinds of antioxidants in combination is permitted. Specific examples of antioxidants preferably include those that are solid at room temperature, and examples thereof include one or more antioxidants selected from the group consisting of ascorbic acid, ascorbate, erythorbic acid, and erythorbate.

The counterion of each of ascorbate and erythorbate is not limited, but can be one or more metal salts, each independently selected from the group consisting of sodium salts, potassium salts, calcium salts, and magnesium salts.

As an antioxidant, the use of an antioxidant acceptable in foods or pharmaceuticals is particularly preferred. As the antioxidant, an ascorbate is preferred, and it is particularly preferred that the ascorbate be one or more selected from sodium ascorbate and calcium ascorbate.

The reduced coenzyme Q10 composition according to the present embodiment preferably further contains a binder. The binder can be utilized to bind respective components such as reduced coenzyme Q10, an emulsifier, an antioxidant, and the like, and to form a composition in a solid form.

The type of binder is not limited. Use may be made of two or more binders in combination. Specific examples of binders include one or more selected from celluloses and starches.

Examples of the celluloses include hydroxypropyl cellulose, hydroxypropyl methylcellulose, hydroxymethyl cellulose, carboxymethyl cellulose, crystalline cellulose, cellulose powder, methyl cellulose, ethyl cellulose, and salts thereof and the like. A particularly preferred binder is one or more selected from the group consisting of hydroxypropyl cellulose, hydroxypropyl methylcellulose, and sodium carboxymethyl cellulose.

Examples of the starches include wheat starch, potato starch, sweet potato starch, corn starch, dextrin, hydroxypropyl starch, acetate starch, oxidized starch, partially alpha-converted starch, and the like.

As a binder, the use of a binder that is acceptable as a food or a pharmaceutical is particularly preferred.

In the reduced coenzyme Q10 composition according to the present embodiment, the blending proportion of each component can be appropriately set according to the dosage form and the like.

In a case where the reduced coenzyme Q10 composition according to the present embodiment contains an antioxidant, the composition contains, with respect to 100 parts by weight of reduced coenzyme Q10, for example, 1 part by weight or more and 9900 parts by weight or less of the antioxidant. The lower limit of the content of the antioxidant with respect to 100 parts by weight of reduced coenzyme Q10 is preferably 20 parts by weight or more, more preferably 50 parts by weight or more, more preferably 55 parts by weight or more. The upper limit of the content of an antioxidant with respect to 100 parts by weight of reduced coenzyme Q10 is preferably 5000 parts by weight or less, more preferably 1000 parts by weight or less, more preferably 500 parts by weight or less, more preferably 200 parts by weight or less, and more preferably 120 parts by weight or less. The content of the antioxidant with respect to 100 parts by weight of reduced coenzyme Q10 is within the range of preferably 20 to 5000 parts by weight, more preferably 50 to 1000 parts by weight, and particularly preferably 55 to 500 parts by weight.

In the case where the reduced coenzyme Q10 composition according to the present embodiment contains an emulsifier, the composition contains, with respect to 100 parts by weight of reduced coenzyme Q10, for example, from 1 part by weight or more to 9900 parts by weight or less of the emulsifier. The lower limit of the content of the emulsifier with respect to 100 parts by weight of reduced coenzyme Q10 is preferably 3 parts by weight or more, more preferably 5 parts by weight or more, and more preferably 8 parts by weight or more. The upper limit of the content of the emulsifier with respect to 100 parts by weight of reduced coenzyme Q10 is preferably in a proportion of 5000 parts by weight or less, more preferably 1000 parts by weight or less, more preferably 500 parts by weight or less, more preferably 200 parts by weight or less, more preferably 150 parts by weight or less, more preferably 100 parts by weight or less, more preferably 50 parts by weight or less, more preferably 30 parts by weight or less. That is, the content of an emulsifier with respect to 100 parts by weight of reduced coenzyme Q10 can be within the range of preferably 3 to 5000 parts by weight, more preferably 5 to 1000 parts by weight, and particularly preferably 8 to 500 parts by weight.

In the reduced coenzyme Q10 composition according to the present embodiment, the lower limit of the content of the antioxidant is, for example, 1 wt % or more, preferably 2 wt % or more, more preferably 4 wt % or more, more preferably 5 wt % or more, and the upper limit of the content is, for example, 99 wt % or less, preferably 90 wt % or less, preferably 80 wt % or less, more preferably 70 wt % or less, more preferably 60 wt % or less. That is, the content of the antioxidant can be, for example, within the range of 1 to 99 wt %, preferably within the range of 2 to 90 wt %, more preferably within the range of 4 to 80 wt %, and particularly preferably within the range of 5 to 60 wt %.

In the reduced coenzyme Q10 composition according to the present embodiment, the content of the emulsifier is not particularly limited, but the lower limit of the content of the emulsifier is, for example, 0.5 wt % or more, preferably 0.7 wt % or more, more preferably 0.9 wt % or more, and the upper limit of the content is, for example, 99 wt % or less, preferably 50 wt % or less, more preferably 30 wt % or less, more preferably 25 wt % or less, more preferably 20 wt % or less, more preferably 15 wt % or less, more preferably 10 wt % or less. Namely, the content of the emulsifier can be, for example, within the range of 0.5 to 99 wt %, preferably 0.7 to 50 wt %, more preferably 0.9 to 10 wt %.

In the case where the reduced coenzyme Q10 composition according to the present embodiment contains a binder, the lower limit of the content of the binder in the composition is, for example, 1 wt % or more, preferably 10 wt % or more, more preferably 20 wt % or more, and the upper limit of the content is, for example, 80 wt % or less, preferably 70 wt % or less, more preferably 60 wt % or less. That is, the content of the binder can be, for example, within the range of 1 to 80 wt %, preferably 10 to 70 wt %, more preferably 20 to 60 wt %.

The reduced coenzyme Q10 composition according to the present embodiment more preferably contains, with respect to 100 parts by weight of reduced coenzyme Q10, a binder in a proportion of, for example, 1 part by weight or more, preferably 3 parts by weight or more, more preferably 5 parts by weight or more, particularly preferably 8 parts by weight or more, and still more preferably 10 parts by weight or more. Also, the composition contains, with respect to 100 parts by weight of reduced coenzyme Q10, an antioxidant in a proportion of, for example, 600 parts by weight or less, preferably 550 parts by weight or less, more preferably 500 parts by weight or less, more preferably 450 parts by weight or less, and still more preferably 400 parts by weight or less.

In a reduced coenzyme Q10 composition according to one or more embodiments of the present invention, the average particle diameter or median diameter (D50) of particles containing reduced coenzyme Q10 (hereinafter sometimes simply referred to as “particles”) is not particularly limited. In one embodiment, with respect to the average particle diameter or D50 of the particles, the average particle diameter in the particle size distribution on a volume basis obtained by measurement using the laser diffraction/scattering method under wet conditions, is typically 40 μm or more, preferably 45 μm or more, more preferably 50 μm or more, more preferably 55 μm or more, more preferably 60 μm or more, more preferably 65 μm or more, and typically 100 μm or less, preferably 95 μm or less, more preferably 90 μm or less, that is, typically within the range of 40 μm to 100 μm, preferably 45 μm to 95 μm, more preferably 50 μm to 90 μm, more preferably 55 μm to 90 μm, more preferably 60 μm to 90 μm, more preferably 65 μm to 90 μm, and, D50 in the particle size distribution on a volume basis obtained by measurement using the laser diffraction/scattering method under wet conditions, is typically 30 μm or more, preferably 35 μm or more, more preferably 40 μm or more, more preferably 45 μm or more, and typically 80 μm or less, preferably 70 μm or less, more preferably 60 μm or less, more preferably 55 μm or less, that is, typically within the range of 30 μm to 80 μm, more preferably 35 μm to 70 μm, more preferably 40 μm to 60 μm, more preferably 45 μm to 55 μm. By the average particle diameter or D50 of the particles falling within the above range, with respect to reduced coenzyme Q10, transition to the other crystal form is more suppressed and stability improves, and furthermore the in vivo absorbability of reduced coenzyme Q10 improves. In general, it was known that, if the average particle diameter or D50 of particles becomes smaller, the specific surface area becomes larger, the reactivity of the particles increases, whereas the stability decreases. However, in one or more embodiments of the present invention, by the average particle diameter or D50 of the particle being in the above range, the transition of reduced coenzyme Q10 in the particle to the other crystal form is suppressed as compared with a particle whose average particle diameter or D50 is larger than the above range, and, as a result, the stability improves.

In a reduced coenzyme Q10 composition according to one or more embodiments of the present invention, the particle size distribution of particles containing reduced coenzyme Q10 is not particularly limited. In one embodiment, the particle size distribution of the particles may be bimodal in the particle size distribution on a volume basis obtained by measurement using the laser diffraction/scattering method under wet conditions. Here, bimodal means that, in a particle size distribution, two populations, a large particle population and a small particle population, are present. In the case where the particle size distribution of the particles is bimodal, when the particle diameter corresponding to the peak top of the large particle is defined as a particle diameter d_{L} and the particle diameter corresponding to the peak top of the small particle is defined as a particle diameter d_{S}, d_{L} is not particularly limited, but is typically 100 μm or more, preferably 105 μm or more, preferably 120 μm or more, more preferably 140 μm or more, more preferably 160 μm or more, more preferably 180 μm or more, and is typically 1000 μm or less, preferably 600 μm or less, more preferably 500 μm or less, more preferably 400 μm or less, more preferably 300 μm or less, that is, is within the range of typically 100 μm to 1000 μm, preferably 105 μm to 1000 μm, preferably 120 μm to 600 μm, more preferably 140 μm to 500 μm, more preferably 160 μm to 400 μm, more preferably 180 μm to 300 μm, and d_{S} is not particularly limited, but is typically 10 μm or more, preferably 20 μm or more, preferably 25 μm or more, more preferably 30 μm or more, more preferably 40 μm or more, more preferably 50 μm or more, and is typically less than 100 μm, preferably 99 μm or less, more preferably 90 μm or less, more preferably 80 μm or less, more preferably 70 μm or less, more preferably 60 μm or less, that is, is within the range of typically 10 μm or more and less than 100 μm, preferably 10 μm to 99 μm, more preferably 20 μm to 90 μm, more preferably 25 μm to 90 μm, more preferably 30 μm to 80 μm, more preferably 40 μm to 70 μm, more preferably 50 μm to 60 μm, and the particle diameter ratio (d_{L}/d_{S}) is not particularly limited, but is typically 1.1 or more, preferably 1.3 or more, more preferably 2 or more, more preferably 3 or more, and is typically 100 or less, preferably 30 or less, more preferably 10 or less, more preferably 6 or less, that is, is within the range of typically 1.1 to 100, preferably 1.3 to 30, more preferably 2 to 10, more preferably 3 to 6. By the particle size distribution of the particles being within the above-described range, with respect to reduced coenzyme Q10, the transition to other crystal forms is more suppressed and stability improves, and furthermore the in vivo absorbability of reduced coenzyme Q10 improves. Generally, in a particle size distribution of particles, in a case where a population of small particles is present, it has been known that the specific surface area is increased by the small particles and the reactivity of the particles increases, whereas the stability decreases. However, in one or more embodiments of the present invention, by the particle size distribution of the particle being within the range, the transition of reduced coenzyme Q10 in the particle to the other crystal form is suppressed as compared with that in particles in which the particle size distribution is unimodal, and, as a result, stability improves.

Preferably, the frequency corresponding to the peak top of the small particle is higher than the frequency corresponding to the peak top of the large particle. The ratio of the frequency corresponding to the peak top of the small particle to the frequency corresponding to the peak top of the large particle (the frequency corresponding to the peak top of the small particle/the frequency corresponding to the peak top of the large particle) may be 0.3 or more, 0.4 or more, 0.5 or more, 0.6 or more, 0.7 or more, 0.8 or more, 0.9 or more, 1.0 or more, 1.1 or more, 1.2 or more, 1.3 or more, 1.4 or more, 1.5 or more, 1.6 or more, or 1.7 or more. The ratio of the frequency corresponding to the peak top of the small particle to the frequency corresponding to the peak top of the large particle (the frequency corresponding to the peak top of the small particle/the frequency corresponding to the peak top of the large particle) may be 10 or less, 9.0 or less, 8.0 or less, 7.0 or less, 6.0 or less, 5.0 or less, 4.0 or less, 3.5 or less, 3.0 or less, 2.5 or less, 2.0 or less, 1.9 or less, or 1.8 or less. The ratio of the frequency corresponding to the peak top of small particles to the frequency corresponding to the peak top of large particles (the frequency corresponding to the peak top of small particles/the frequency corresponding to the peak top of large particles) may be 0.3 to 10, 0.4 to 10, 0.5 to 10, 0.6 to 10, 0.7 to 10, 0.8 to 10, 0.9 to 10, 1.0 to 10, 1.1 to 10, 1.2 to 10, 1.3 to 10, 1.4 to 10, 1.5 to 10, 1.6 to 10, 1.7 to 10, 1.7 to 9.0, 1.7 to 8.0, 1.7 to 7.0, 1.7 to 6.0, 1.7 to 5.0, 1.7 to 4.0, 1.7 to 3.5, 1.7 to 3.0, 1.7 to 2.5, 1.7 to 2.0, 1.7 to 1.9, or 1.7 to 1.8.

In a reduced coenzyme Q10 composition according to one or more embodiments of the present invention, when the composition is in solid form, the angle of repose of the composition is not particularly limited. The angle of repose of the composition is typically 48 degrees or more, preferably 49 degrees or more, more preferably 50 degrees or more, and is typically 55 degrees or less, preferably 54 degrees or less, more preferably 53 degrees or less, that is, it is typically within the range of 48 to 55 degrees, preferably 49 to 54 degrees, more preferably 50 to 53 degrees. When the angle of repose of the composition is within the above range, the flowability of the composition becomes favorable, and the handleability improves.

In a reduced coenzyme Q10 composition according to one or more embodiments of the present invention, when the composition is in solid form, the bulk density of the composition is not particularly limited. The bulk density of the composition is typically 0.190 g/ml or more, preferably 0.195 g/ml or more, more preferably 0.198 g/ml or more, and is typically 0.250 g/ml or less, preferably 0.240 g/ml or less, more preferably 0.230 g/ml or less, and more preferably 0.220 g/ml or less, that is, it is typically within the range of 0.190 g/ml to 0.250 g/ml, preferably 0.195 g/ml to 0.240 g/ml, more preferably 0.198 g/ml to 0.230 g/ml, and more preferably 0.198 g/ml to 0.220 g/ml. When the bulk density of the composition is within the above range, the fillability of the composition favorably improves, and the handleability improves.

In a case where a reduced coenzyme Q10 composition according to one or more embodiments of the present invention contains Form I crystal, when Form II type reduced coenzyme Q10 crystal in an amount of 10 wt % with respect to the Form I crystal contained in the composition and 99.5% ethanol are added to the composition, and the mixture is stirred and then allowed to stand at 37° C. for a time of 6 hours, the proportion of the Form II crystal in the mixture, in a measurement by a differential scanning calorimeter (DSC), as the proportion (%) of the endothermic peak area of the Form II crystal to the total of the endothermic peak area of the Form I crystal and the endothermic peak area of the Form II crystal, is typically 25% or less, in one embodiment, 24% or less, in one embodiment, 23% or less, in one embodiment, 22% or less, in one embodiment, 21% or less, in one embodiment, 20% or less, in one embodiment, 19% or less, in one embodiment, 18% or less, in one embodiment, 17% or less, in one embodiment, 16% or less, in one embodiment, 15% or less, in one embodiment, 14% or less, in one embodiment, 13% or less, in one embodiment, 12% or less, in one embodiment, 11% or less, in one embodiment, 10% or less, in one embodiment, 9% or less, and in one embodiment, 8% or less. The lower limit value of the proportion is not limited, but the proportion is typically 1% or more.

In a case where the reduced coenzyme Q10 composition according to one or more embodiments of the present invention contains Form I crystal, when Form II type reduced coenzyme Q10 crystal in an amount of 10 wt % with respect to the Form I crystal contained in the composition and 99.5% ethanol are added to the composition, and the mixture is stirred and then allowed to stand at 37° C. for a time of 6 hours, the fact that the proportion of Form II crystal in the mixture is small means that the transition rate from Form I crystal to Form II crystal of the reduced coenzyme Q10 composition according to one or more embodiments of the present invention is small, and therefore means that the polymorphic stability of the crystal of the reduced coenzyme Q10 composition according to one or more embodiments of the present invention is high. When the polymorphic stability of the crystal of the reduced coenzyme Q10 composition is high, quality change due to transition of crystal polymorphism is less likely to occur, and it is more preferred in terms of quality control.

Next, the production method of the reduced coenzyme Q10 composition of one or more embodiments of the present invention will be described. In one or more embodiments of the present invention, the reduced coenzyme Q10 composition can be produced by separation of a composition in solid form that contains reduced coenzyme Q10, reduced demethoxy Q10, and reduced coenzyme Q9 from a solution containing the reduced coenzyme Q10, reduced demethoxy Q10, and reduced coenzyme Q9. When separating the composition in solid form, by controlling the blending ratio of each component in the solution and the conditions of the separation step so that the peak area ratio in the chromatogram for RD and RQ9 in the composition is within the specific range, the composition in which polymorphic transition of reduced coenzyme Q10 (QH) is unlikely to occur can be obtained. Specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be controlled to be 0.3 or more. The separation step may be carried out so that the above peak area ratio (RD/RQ9) is 0.5 or more, 0.8 or more, 1.0 or more, 1.5 or more, or 2.0 or more. The upper limit of the above peak area (RD/RQ9) is not particularly limited, but, the separation step may be performed so that the above peak area ratio (RD/RQ9) may be 10.0 or less, 8.0 or less, 6.0 or less, 4.0 or less, or 3.0 or less. That is, the above peak area ratio may be 0.3 or more, may be within the range of 0.3 to 10.0, 0.5 to 10.0, 0.5 to 3.0, 0.8 to 8.0, 1.0 to 6.0, 1.0 to 2.0, 1.5 to 4.0, or 2.0 to 3.0.

In another embodiment, specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be controlled to be 0.3 or more. The above peak area ratio (RD/RQ9) may be, for example, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, or in one embodiment, 2.0 or more. The upper limit of the above peak area ratio (RD/RQ9) is not particularly limited, but may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.0 or less, in one embodiment, 2.8 or less, in one embodiment, 2.6 or less, in one embodiment, 2.4 or less, in one embodiment, 2.2 or less, or in one embodiment, 2.1 or less. That is, the above peak area ratio may be 0.3 or more, and, for example, may be within the range of 0.3 to 10.0, in one embodiment, 0.3 to 9.0, in one embodiment, 0.3 to 8.0, in one embodiment, 0.3 to 7.0, in one embodiment, 0.3 to 6.0, in one embodiment, 0.3 to 5.0, in one embodiment, 0.3 to 4.0, in one embodiment, 0.3 to 3.0, in one embodiment, 0.3 to 2.8, in one embodiment, 0.3 to 2.6, in one embodiment, 0.3 to 2.4, in one embodiment, 0.3 to 2.2, in one embodiment, 0.3 to 2.1, in one embodiment, 0.4 to 2.1, in one embodiment, 0.5 to 2.1, in one embodiment, 0.6 to 2.1, in one embodiment, 0.7 to 2.1, in one embodiment, 0.8 to 2.1, in one embodiment, 0.9 to 2.1, in one embodiment, 1.0 to 2.1, in one embodiment, 1.1 to 2.1, in one embodiment, 1.2 to 2.1, in one embodiment, 1.3 to 2.1, in one embodiment, 1.4 to 2.1, in one embodiment, 1.5 to 2.1, in one embodiment, 1.6 to 2.1, in one embodiment, 1.7 to 2.1, in one embodiment, 1.8 to 2.1, in one embodiment, 1.9 to 2.1, in one embodiment, 2.0 to 2.1, in one embodiment, 0.5 to 10.0, in one embodiment, 0.5 to 3.0, in one embodiment, 0.8 to 8.0, in one embodiment, 1.0 to 6.0, in one embodiment, 1.0 to 2.0, in one embodiment, 1.5 to 4.0, or in one embodiment, 2.0 to 3.0.

The separation step, in one embodiment, can be performed by adding a seed crystal of reduced coenzyme Q10 to a solution containing reduced coenzyme Q10, reduced demethoxy Q10, and reduced coenzyme Q9, and cooling the solution. Thereby, it is possible to cause precipitation of the reduced coenzyme Q10 crystal and to obtain a composition in solid form.

A solution containing reduced coenzyme Q10, reduced demethoxy Q10, and reduced coenzyme Q9 may be a homogeneous solution state with dissolution of reduced coenzyme Q10 and the like in an organic solvent, or may be a slurry state with a part remaining without dissolution, however, a homogeneous solution state is preferred. In addition, the solution may contain various compounds in addition to the above reduced coenzyme Q10, reduced demethoxy Q10, and reduced coenzyme Q9, and examples include QH analogs such as reduced coenzyme Q11 and decaprenylphenol.

It is to be noted that, as for the reduced coenzyme Q10 for use in the above solution, there is no limitation as to whether it is in a crystal state or an amorphous state, and its crystal polymorphism is also not limited. Therefore, the use of conventionally known Form I type reduced coenzyme Q10 is also possible. Also, since it is possible to increase its purity in crystal precipitation, it may be one having analogs, or unpurified or crudely purified reduced coenzyme Q10. Furthermore, an extract solution of reduced coenzyme Q10 obtained by conventionally known methods, and/or a reaction solution that contains reduced coenzyme Q10 obtained from oxidized coenzyme Q10 by a known reduction method, as is, or, if necessary, after purification and/or solvent substitution, can be used as the solution in the separation step. For example, in a solution in which oxidized coenzyme Q10 and reduced coenzyme Q10 are present together, a solution obtained after subjecting only oxidized coenzyme Q10 to reduction treatment by a known reduction method, or, in a solution containing only oxidized coenzyme Q10, a solution obtained after subjecting oxidized coenzyme Q10 to reduction treatment by a known reduction method, or, in a solution in which oxidized coenzyme Q10 and components other than coenzyme Q10 are present together, a solution obtained after subjecting only oxidized coenzyme Q10 to reduction treatment by a known reduction method, or the like, can be used as a solution in the subsequent separation step.

For example, in the first aspect, the second aspect, and the third aspect of one or more embodiments of the present invention, the solution for the separation step or the precipitation step can be produced by reducing a raw material composition containing oxidized coenzyme Q10 (QX) and at least one selected from the group consisting of oxidized coenzyme Q9 (Q9), oxidized coenzyme Q11 (Q11), oxidized demethoxy Q10 (D), and decaprenylphenol (decaprenyl). The content of each component of the raw material composition can be adjusted so that the peak area ratio in the chromatogram of each component in the raw material composition is within the specific range described below. Accordingly, it is possible to obtain a composition in which the polymorphic transition of reduced coenzyme Q10 (QH) is unlikely to occur and polymorphic stability is improved, and furthermore, a state of coexistence or an oral composition in which the in vivo absorbability, particularly digestive fluid solubility, of reduced coenzyme Q10 (QH) is improved.

For example, in the chromatogram of the raw material composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 275 nm,
   an area ratio (D/Q9) of a peak containing D to a peak containing Q9 is not particularly limited, but is typically 0.15 or more, in one embodiment, 0.30 or more, in one embodiment, 0.40 or more, in one embodiment, 0.50 or more, and in one embodiment, 0.60 or more, and is typically 2.0 or less, in one embodiment, 1.9 or less, in one embodiment, 1.8 or less, in one embodiment, 1.7 or less, and in one embodiment, 1.6 or less, and, for example, may be 0.15 to 2.0, in one embodiment, 0.30 to 1.9, in one embodiment, 0.40 to 1.8, in one embodiment, 0.50 to 1.7, and in one embodiment 0.60 to 1.6.

In the chromatogram of the raw material composition obtained by high performance liquid chromatography under the above conditions, the area ratio (decaprenyl/Q9) of a peak containing decaprenyl to a peak containing Q9 is not particularly limited, but is typically 0.01 or more, in one embodiment, 0.05 or more, in one embodiment, 0.10 or more, in one embodiment, 0.12 or more, in one embodiment, 0.14 or more, and may typically be 1.0 or less, in one embodiment, 0.80 or less, in one embodiment, 0.70 or less, in one embodiment, 0.60 or less, in one embodiment, 0.50 or less, and may be, for example, 0.01 to 1.0, in one embodiment, 0.05 to 0.80, in one embodiment, 0.10 to 0.70, in one embodiment, 0.12 to 0.60, and in one embodiment, 0.14 to 0.50.

In the chromatogram of the raw material composition obtained by high performance liquid chromatography under the above conditions, the area ratio (D/Q11) of a peak containing D to a peak containing Q11 is not particularly limited, but is typically 0.51 or more, in one embodiment, 0.54 or more, in one embodiment, 0.56 or more, in one embodiment, 0.58 or more, in one embodiment, 0.60 or more, and may be typically 1.0 or less, in one embodiment, 0.90 or less, in one embodiment, 0.80 or less, in one embodiment, 0.75 or less, and in one embodiment, 0.70 or less, for example may be 0.51 to 1.0, in one embodiment, 0.54 to 0.90, in one embodiment, 0.56 to 0.80, in one embodiment, 0.58 to 0.75, and in one embodiment 0.60 to 0.70.

In the chromatogram of the raw material composition obtained by high performance liquid chromatography under the above conditions, the area ratio (decaprenyl/Q11) of a peak containing decaprenyl and a peak containing Q11 is not particularly limited, but is typically 0.01 or more, in one embodiment, 0.05 or more, in one embodiment, 0.10 or more, in one embodiment, 0.12 or more, in one embodiment, 0.13 or more, and may be typically 1.0 or less, in one embodiment, 0.60 or less, in one embodiment, 0.50 or less, in one embodiment, 0.40 or less, in one embodiment, 0.30 or less, and, for example, may be 0.01 to 1.0, in one embodiment, 0.05 to 0.60, in one embodiment, 0.10 to 0.50, in one embodiment, 0.12 to 0.40, and in one embodiment, 0.13 to 0.30.

In the chromatogram of the raw material composition obtained by high performance liquid chromatography under the above conditions, the area ratio (Q11/Q9) of a peak containing Q11 to a peak containing Q9 is not particularly limited, but is typically 0.25 or more, in one embodiment, 0.40 or more, in one embodiment, 0.60 or more, in one embodiment, 0.80 or more, and in one embodiment 1.0 or more, and may be typically 5.0 or less, in one embodiment, 4.5 or less, in one embodiment, 4.0 or less, in one embodiment, 3.5 or less, and in one embodiment 3.0 or less, for example, may be 0.25 to 5.0, in one embodiment, 0.40 to 4.5, in one embodiment, 0.60 to 4.0, in one embodiment, 0.80 to 3.5, and in one embodiment 1.0 to 3.0.

In the chromatogram of the raw material composition obtained by high performance liquid chromatography under the above conditions, the area ratio (Q9/QX) of a peak containing Q9 to a peak containing QX is not particularly limited, but is typically 0.0001 or more, in one embodiment, 0.0002 or more, in one embodiment, 0.0005 or more, in one embodiment, 0.0008 or more, in one embodiment, 0.0010 or more, and is typically 0.0026 or less, in one embodiment, 0.0025 or less, in one embodiment, 0.0023 or less, in one embodiment, 0.0021 or less, in one embodiment, 0.0020 or less, and may be, for example, 0.0001 to 0.0026, in one embodiment, 0.0002 to 0.0025, in one embodiment, 0.0005 to 0.0023, in one embodiment, 0.0008 to 0.0021, and in one embodiment, 0.0010 to 0.0020.

In the chromatogram of the raw material composition obtained by high performance liquid chromatography under the above conditions, the area ratio (D/QX) of a peak containing D to a peak containing QX is not particularly limited, but is typically 0.00032 or more, in one embodiment, 0.00040 or more, in one embodiment, 0.00060 or more, in one embodiment, 0.00080 or more, in one embodiment, 0.0010 or more, and may typically be 0.010 or less, in one embodiment, 0.0080 or less, in one embodiment, 0.0060 or less, in one embodiment, 0.0040 or less, and in one embodiment, 0.0020 or less, for example, may be 0.00032 to 0.010, in one embodiment, 0.00040 to 0.0080, in one embodiment, 0.00060 to 0.0060, in one embodiment, 0.00080 to 0.0040, and in one embodiment 0.0010 to 0.0020.

In the chromatogram of the raw material composition obtained by high performance liquid chromatography under the above conditions, the area ratio (decaprenyl/QX) of a peak containing decaprenyl to a peak containing QX is not particularly limited, but is typically 0.00010 or more, in one embodiment, 0.00013 or more, in one embodiment, 0.00016 or more, in one embodiment, 0.00018 or more, in one embodiment, 0.00020 or more, and may typically be 0.010 or less, in one embodiment, 0.0050 or less, in one embodiment, 0.0010 or less, in one embodiment, 0.00080 or less, in one embodiment, 0.00060 or less, and, for example, may be 0.00010 to 0.010, in one embodiment, 0.00013 to 0.0050, in one embodiment, 0.00016 to 0.0010, in one embodiment, 0.00018 to 0.00080, and in one embodiment, 0.00020 to 0.00060.

In the chromatogram of the raw material composition obtained by high performance liquid chromatography under the above conditions, the area ratio (Q11/QX) of a peak containing Q11 and a peak containing QX is not particularly limited, but is typically 0.00063 or more, in one embodiment, 0.00070 or more, in one embodiment, 0.0010 or more, in one embodiment, 0.0015 or more, in one embodiment, 0.0020 or more, and is typically 0.10 or less, in one embodiment, 0.050 or less, in one embodiment, 0.010 or less, in one embodiment, 0.0050 or less, in one embodiment, 0.0030 or less, and may be, for example, 0.00063 to 0.10, in one embodiment, 0.00070 to 0.050, in one embodiment, 0.0010 to 0.010, in one embodiment, 0.0015 to 0.0050, and in one embodiment, 0.0020 to 0.0030.

In the present embodiment, as an organic solvent for use in a solution containing reduced coenzyme Q10, reduced demethoxy Q10, and reduced coenzyme Q9, it is preferred to use at least one organic solvent selected from the group consisting of alcohol, hydrocarbon, fatty acid ester, and nitrogen compound.

As the alcohol to be used as the organic solvent, regardless of whether cyclic alcohols or acyclic alcohols and, further, regardless of whether saturated alcohols or unsaturated alcohols, it is not particularly limited, but, in general, saturated alcohols are preferably used. For example, typically, a monohydric alcohol having 1 or more carbon atoms, in one embodiment, having 2 or more carbon atoms, typically a monohydric alcohol having 20 or less carbon atoms, in one embodiment, having 12 or less carbon atoms, in one embodiment, having 6 or less carbon atoms, in one embodiment, having 5 or less carbon atoms, in one embodiment, having 4 or less carbon atoms, in one embodiment, having 3 or less carbon atoms is preferred, or typically, a dihydric alcohol having 2 or more carbon atoms, typically having 5 or less carbon atoms is preferred, or a trihydric alcohol having 3 carbon atoms is preferred. For example, a monohydric alcohol having 1 to 20 carbon atoms, 1 to 12 carbon atoms, particularly 1 to 6 carbon atoms, more particularly 1 to 5 carbon atoms, still more particularly 1 to 4 carbon atoms, even more particularly 1 to 3 carbon atoms, and still further having 2 to 3 carbon atoms is preferred, or a dihydric alcohol having 2 to 5 carbon atoms is preferred, or a trihydric alcohol having 3 carbon atoms is preferred. Among these, a monohydric alcohol of 1 to 5 carbon atoms is an alcohol having high compatibility with water and is preferably used in the case of use as a mixed solvent with water.

Examples of the monohydric alcohols can include methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol, 4-methylcyclohexanol, and the like.

The monohydric alcohol is preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, and cyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, and neopentyl alcohol, still more preferably methanol, ethanol, 1-propanol, 2-propanol, 1-butanol, 2-butanol, isobutyl alcohol, 2-methyl-1-butanol, and isopentyl alcohol, in particular, methanol, ethanol, 1-propanol, and 2-propanol, further preferably, ethanol, 1-propanol, and 2-propanol, and most preferably ethanol.

Examples of the dihydric alcohols include 1,2-ethanediol, 1,2-propanediol, 1,3-propanediol, 1,2-butanediol, 1,3-butanediol, 1,4-butanediol, 2,3-butanediol, 1,5-pentanediol, and the like. Preferably, the dihydric alcohol is 1,2-ethanediol, 1,2-propanediol, or 1,3-propanediol, most preferably, 1,2-ethanediol.

As a trihydric alcohol, glycerin or the like can preferably be used.

The hydrocarbon is not particularly limited, and examples thereof include an aliphatic hydrocarbon, an aromatic hydrocarbon, a halogenated hydrocarbon, and the like.

As the aliphatic hydrocarbon, regardless of whether cyclic aliphatic hydrocarbons or acyclic aliphatic hydrocarbons, and regardless of whether saturated aliphatic hydrocarbons or unsaturated aliphatic hydrocarbons, it is not particularly limited, but typically the aliphatic hydrocarbons having 3 or more carbon atoms, in one embodiment the aliphatic hydrocarbons having 5 or more carbon atoms, typically the aliphatic hydrocarbons having 20 or less carbon atoms, and in one embodiment the aliphatic hydrocarbons having 12 or less carbon atoms are used. The aliphatic hydrocarbon is not particularly limited regardless of whether cyclic aliphatic hydrocarbons or acyclic aliphatic hydrocarbons, and regardless of whether saturated aliphatic hydrocarbons or unsaturated aliphatic hydrocarbons, and typically the aliphatic hydrocarbons having 3 to 20 carbon atoms, preferably 5 to 12 carbon atoms, are used. Specific examples include propane, butane, isobutane, pentane, 2-methylbutane, cyclopentane, 2-pentene, hexane, 2-methylpentane, 2,2-dimethylbutane, 2,3-dimethylbutane, methylcyclopentane, cyclohexane, 1-hexene, cyclohexene, heptane, 2-methylhexane, 3-methylhexane, 2,3-dimethylpentane, 2,4-dimethylpentane, methylcyclohexane, 1-heptene, octane, 2,2,3-trimethylpentane, isooctane, ethylcyclohexane, 1-octene, nonane, 2,2,5-trimethylhexane, 1-nonene, decane, 1-decene, p-menthane, undecane, dodecane, and the like. Preferable examples are pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, nonane, 2,2,5-trimethylhexane, decane, dodecane, cyclopentane, methylcyclopentane, cyclohexane, methylcyclohexane, ethylcyclohexane, p-menthane, and the like. More preferable examples are pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, cyclopentane, methylcyclopentane, cyclohexane, methylcyclohexane, ethylcyclohexane, and the like, even more preferable examples are pentane, hexane, cyclohexane, methylcyclohexane, and the like, particularly preferable examples are heptane, hexane, methylcyclohexane, and most preferable examples are heptane and hexane.

The aromatic hydrocarbon is not particularly limited, but typically the aromatic hydrocarbons having 6 or more carbon atoms, in one embodiment, the aromatic hydrocarbons having 7 or more carbon atoms, typically the aromatic hydrocarbons having 20 or less carbon atoms, in one embodiment, the aromatic hydrocarbons having 12 or less carbon atoms, and in one embodiment the aromatic hydrocarbons having 10 or less carbon atoms are used. The aromatic hydrocarbon is not particularly limited, but typically the aromatic hydrocarbons having 6 to 20 carbon atoms, preferably 6 to 12 carbon atoms, more preferably 7 to 10 carbon atoms are used. Specific examples include benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene, styrene, and the like.

The halogenated hydrocarbon is not particularly limited regardless of whether cyclic halogenated hydrocarbons or acyclic halogenated hydrocarbons and regardless of whether saturated halogenated hydrocarbons or unsaturated halogenated hydrocarbons, but acyclic ones are preferably used. Chlorinated hydrocarbons and fluorinated hydrocarbons are more preferred, and chlorinated hydrocarbons are further preferred.

As the halogenated hydrocarbon, preferably the halogenated hydrocarbons having 1 or more carbon atoms, preferably the halogenated hydrocarbons having 6 or less carbon atoms, more preferably the halogenated hydrocarbons having 4 or less carbon atoms, and more preferably the halogenated hydrocarbons having 2 or less carbon atoms are used. Further, as the halogenated hydrocarbon, preferably the halogenated hydrocarbons having 1 to 6 carbon atoms, more preferably the halogenated hydrocarbons having 1 to 4 carbon atoms, and more preferably the halogenated hydrocarbons having 1 to 2 carbon atoms are used. Specific examples include dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2-tetrachloroethane, pentachloroethane, hexachloroethane, 1,1-dichloroethylene, 1,2-dichloroethylene, trichloroethylene, tetrachloroethylene, 1,2-dichloropropane, 1,2,3-trichloropropane, chlorobenzene, and 1,1,1,2-tetrafluoroethane, and the like.

The fatty acid esters are not particularly limited, and examples thereof include propionic acid esters (propionate), acetic acid esters (acetate), formic acid esters (formate), and the like. Acetic acid esters and formic acid esters are preferred, and acetic acid esters are more preferred.

The ester group is not particularly limited, and examples thereof include an alkyl ester having 1 or more carbon atoms, an alkyl ester having 8 or less carbon atoms, an aralkyl ester having 1 or more carbon atoms, and, typically, an aralkyl ester having 8 or less carbon atoms, preferably having 6 or less carbon atoms, more preferably having 4 or less carbon atoms, and the like. The ester group is not particularly limited, and examples thereof include an alkyl ester having 1 to 8 carbon atoms, an aralkyl ester having 1 to 8 carbon atoms, and the like. The ester group is preferably an alkyl ester having 1 to 6 carbon atoms, and more preferably an alkyl ester having 1 to 4 carbon atoms.

Examples of the propionic acid esters include methyl propionate, ethyl propionate, butyl propionate, isopentyl propionate, and the like.

Examples of the acetic acid esters include methyl acetate, ethyl acetate, propyl acetate, isopropyl acetate, butyl acetate, isobutyl acetate, sec-butyl acetate, pentyl acetate, isopentyl acetate, sec-hexyl acetate, cyclohexyl acetate, benzyl acetate, and the like. Preferred examples include methyl acetate, ethyl acetate, propyl acetate, isopropyl acetate, butyl acetate, isobutyl acetate, and the like, and most preferred is ethyl acetate.

Examples of the formic acid esters include methyl formate, ethyl formate, propyl formate, isopropyl formate, butyl formate, isobutyl formate, sec-butyl formate, pentyl formate, and the like.

As a nitrogen compound, for example, a nitrile can be used. As for the nitrile, the nitrile is not particularly limited regardless of whether cyclic nitriles or acyclic nitriles, and regardless of whether saturated nitriles or unsaturated nitriles, but saturated nitriles are preferably used. Typically, nitriles having 2 or more carbon atoms, typically nitriles having 20 or less carbon atoms, preferably having 12 or less carbon atoms, more preferably having 8 or less carbon atoms, are used. Typically, nitriles having 2 to 20 carbon atoms, preferably having 2 to 12 carbon atoms, more preferably having 2 to 8 carbon atoms, are used. Specific examples of the nitrile include acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chloropropionitrile, bromopropionitrile, methoxyacetonitrile, methyl cyanoacetate, ethyl cyanoacetate, tolunitrile, benzonitrile, chlorobenzonitrile, bromobenzonitrile, cyanobenzoic acid, nitrobenzonitrile, anisnitrile, phthalonitrile, bromotolunitrile, methyl cyanobenzoate, methoxybenzonitrile, acetylbenzonitrile, naphthonitrile, biphenylcarbonitrile, phenylpropionitrile, phenylbutyronitrile, methyl phenylacetonitrile, diphenylacetonitrile, naphthylacetonitrile, nitrophenylacetonitrile, chlorobenzyl cyanide, cyclopropanecarbonitrile, cyclohexanecarbonitrile, cycloheptanecarbonitrile, phenylcyclohexanecarbonitrile, and tolylcyclohexanecarbonitrile. Preferable examples are acetonitrile, propionitrile, succinonitrile, butyronitrile, isobutyronitrile, valeronitrile, methyl cyanoacetate, ethyl cyanoacetate, benzonitrile, tolunitrile, or chloropropionitrile, more preferable examples are acetonitrile, propionitrile, butyronitrile, or isobutyronitrile, and a most preferable example is acetonitrile.

Examples of the nitrogen compounds other than the above nitrile include nitromethane, triethylamine, pyridine, formamide, N-methylformamide, N,N-dimethylformamide, N,N-dimethylacetamide, N-methylpyrrolidone, and the like.

Among the above organic solvents, an alcohol or a hydrocarbon is preferred, and an alcohol is particularly preferred.

In the present embodiment, as the organic solvent for use in a solution that contains reduced coenzyme Q10 and the like, the ones exemplified above may be used alone, and in order to improve conditions that affect crystal precipitation conditions such as the solubility of each component of reduced coenzyme Q10 and the like, crystal precipitation concentration, yield, slurry physical properties, and crystal physical properties, two or more solvents may also be mixed in an appropriate proportion according to the properties of each solvent and used.

Further, as long as the use of at least one organic solvent selected from the group consisting of the above alcohol, hydrocarbon, fatty acid ester and nitrogen compound is made, the auxiliary use of organic solvents other than those is acceptable. Furthermore, in the present embodiment, “organic solvent” includes organic solvents containing water. When an organic solvent containing water is used, the water content in the organic solvent, based on the total amount of the organic solvent containing water, is preferably 0.01 wt % or more, more preferably 0.1 wt % or more, still more preferably 0.5 wt % or more, preferably 50 wt % or less, more preferably 40 wt % or less, still more preferably 34 wt % or less, and particularly preferably 15 wt % or less. When an organic solvent containing water is used, the water content in the organic solvent, based on the total amount of the organic solvent containing water, is preferably 0.01 to 50 wt %, more preferably 0.1 to 40 wt %, still more preferably 0.5 to 34 wt %, and particularly preferably 0.5 to 15 wt %. When the water content exceeds 50 wt %, there are cases where separation of the organic solvent and water occurs, the solubility of reduced coenzyme Q10 and the like in the organic solvent is greatly reduced, and the crystallization of reduced coenzyme Q10 and the like for the purpose becomes difficult. In the case of use of an organic solvent containing water, it is preferred that the water and the organic solvent are in a homogeneous system, and, from this viewpoint as well, alcohols such as ethanol and fatty acid esters such as ethyl acetate are preferably selected as the organic solvent.

In one or more embodiments of the present invention, the organic solvent is a monohydric alcohol typically having 1 or more carbon atoms and typically having 5 or less carbon atoms that contains 15 wt % or less of water or contains no water, and, more preferably, is a monohydric alcohol typically having 1 or more carbon atoms and typically having 5 or less carbon atoms that contains 8 wt % or less of water or contains no water. In one or more embodiments of the present invention, the organic solvent is a monohydric alcohol having 1 to 5 carbon atoms that contains 15 wt % or less of water or contains no water, more preferably, the organic solvent is a monohydric alcohol having 1 to 5 carbon atoms that contains 8 wt % or less of water or contains no water, particularly preferably, the organic solvent is ethanol that contains 8 wt % or less of water or contains no water, and further preferably, the organic solvent is ethanol that contains 0.1 wt % or more and 8 wt % or less of water.

In the solution containing the organic solvent, the concentrations of reduced coenzyme Q10, reduced demethoxy Q10, and reduced coenzyme Q9 can be appropriately adjusted according to the organic solvent for use and are not particularly limited. For example, the concentration of the total of reduced coenzyme Q10, reduced demethoxy Q10, and reduced coenzyme Q9 in a solution is 80 wt % or less, preferably, 70 wt % or less, more preferably 60 wt % or less, further preferably 50 wt % or less, and particularly preferably 40 wt % or less. Also, from the standpoint of production efficiency, it is preferred that the concentration of reduced coenzyme Q10 and the like in the solution be adjusted to a relatively high concentration, for example, preferably 1 wt % or more, more preferably 5 wt % or more, and particularly preferably 10 wt % or more.

The solution to be used in the step of adding a seed crystal to cause precipitation of the crystal is a supersaturated solution with dissolution at a concentration equal to or higher than the saturation concentration of each component such as reduced coenzyme Q10, more preferably at a temperature of 32° C. or higher, more preferably at a temperature of 43° C. or lower. The solution to be used in the step of adding a seed crystal to cause precipitation of the crystal is, more preferably, a supersaturated solution with dissolution at a concentration equal to or higher than the saturation concentration of each component such as reduced coenzyme Q10 at a temperature within the range of 32 to 43° C. Such a supersaturated solution can be prepared by heating a raw material mixture that contains the organic solvent and the respective components such as reduced coenzyme Q10 to a temperature of 42° C. or higher, 45° C. or higher, more preferably 49° C. or higher, further preferably 70° C. or lower, and particularly preferably 55° C. or lower to effect dissolution of the respective components such as reduced coenzyme Q10, and by cooling the solution after the heating to a temperature lower than the heating temperature and a temperature within the range of 32 to 43° C.

Particularly, in the case where the organic solvent is a monohydric alcohol having 1 to 5 carbon atoms (preferably ethanol) that contains 8 wt % or less of water or contains no water, the total concentration of reduced coenzyme Q10, reduced demethoxy Q10, and reduced coenzyme Q9 in the solution is preferably 5 wt % or more, particularly preferably 10 wt % or more, preferably 50 wt % or less, more preferably 40 wt % or less, more preferably 25 wt % or less, and particularly preferably 20 wt % or less. When the concentration is within the range above, a supersaturated solution of reduced coenzyme Q10 and the like is easy to prepare, and by addition of a seed crystal, precipitation of the crystal is easy.

In the present embodiment, a crystal of reduced coenzyme Q10 is added as a seed crystal to the solution containing the organic solvent and reduced coenzyme Q10, reduced demethoxy Q10 and reduced coenzyme Q9, thereby preparing a mixed liquid to be subjected to the crystal precipitation step. The addition amount (seed crystal addition amount) of the reduced coenzyme Q10 crystal that is the seed crystal is not particularly limited, but is, with respect to the amount of reduced coenzyme Q10 in the solution before addition of the seed crystal, preferably 0.001 wt % or more, more preferably 0.005 wt % or more, particularly preferably 0.01 wt % or more, preferably 30 wt % or less, more preferably 20 wt % or less, and particularly preferably 5 wt % or less. The addition amount (seed crystal addition amount) of the reduced coenzyme Q10 crystal that is the seed crystal is not particularly limited, but is, with respect to the amount of reduced coenzyme Q10 in the solution before addition of the seed crystal, preferably 0.001 to 30 wt %, more preferably 0.005 to 20 wt %, and particularly preferably 0.01 to 5 wt %. Note that the reduced coenzyme Q10 crystal for use as a seed crystal is preferably one that includes the reduced coenzyme Q10 crystal of the Form I type, but it is also acceptable if the reduced coenzyme Q10 crystal of the Form II type or an amorphous form of the reduced coenzyme Q10 is included. A higher purity of the Form I type reduced coenzyme Q10 crystal is preferred, and, for example, the use of a seed crystal containing the Form I type reduced coenzyme Q10 crystal at a concentration of 50 wt % or more, preferably 75 wt % or more, further preferably 80 wt % or more, and more preferably 90 wt % or more, is preferred.

Furthermore, in the present embodiment, the temperature of the solution at the time of addition of the seed crystal is preferably 25° C. or higher, and is preferably 43° C. or lower. In the present embodiment, it is preferred that the temperature of the solution at the time of addition of the seed crystal is within the range of 25 to 43° C. More preferably, the temperature of the solution at the time of addition of the seed crystal is 27° C. or higher, and, particularly preferably, is 29° C. or higher. Further, it is preferred that the upper limit be 41° C. or lower. If the temperature of the solution at the time of addition of the seed crystal exceeds 43° C., there are cases where dissolution of the added seed crystal occurs and precipitation of the crystal does not occur. Further, if a seed crystal is added to a solution at the temperature of less than 25° C., the proportion of the crystal in the reduced coenzyme Q10 obtained by precipitation may become low, or precipitation of the crystal may not occur.

Next, one or more preferred embodiments of the crystal precipitation step that causes precipitation of a reduced coenzyme Q10 crystal in the mixed liquid will be described. If the temperature of the solution at the time of addition of the seed crystal is within the above range, the temperature of the crystal precipitation step after adding the seed crystal is not particularly limited, but the step preferably includes maintaining, for 1 hour or more after adding the seed crystal, the temperature of the mixed liquid at a temperature preferably −1° C. or higher, preferably 41° C. or lower. If the temperature of the solution at the time of addition of the seed crystal is within the above range, the temperature of the crystal precipitation step after adding the seed crystal is not particularly limited, but the step preferably includes maintaining, for 1 hour or more after adding the seed crystal, the temperature of the mixed liquid, preferably at −1° C. or higher, more preferably within the range of −1 to 41° C. The time to maintain the mixed liquid in the temperature range is not particularly limited, but is, preferably 1 hour or more, preferably 2 hours or more, more preferably 4 hours or more, and particularly preferably 10 hours or more. The upper limit of the time to maintain the mixed liquid in the temperature range is not particularly limited, but a sufficient effect can be obtained in about 24 hours. That is, the time to maintain the mixed liquid in the temperature range is preferably within the range of, for example, 1 to 24 hours. In this case, for example, a constant temperature in the range of −1 to 41° C. may be maintained, or, for example, in cooling crystallization or the like, after addition of the seed crystal, the mixed liquid may be allowed to reach −1° C. while being gradually cooled. Further, throughout the crystal precipitation step, it may be permissible to maintain a temperature of −1 to 41° C., or, after maintaining a temperature of −1 to 41° C. for, for example, a time of 1 hour or more, thereafter to perform cooling.

The endpoint temperature of the crystal precipitation step is not particularly limited, but, from the viewpoint of an increase in recovery amount, the crystal precipitation step is preferably carried out at an endpoint temperature of 35° C. or lower, more preferably 30° C. or lower, and particularly 25° C. or lower. The lower limit is the solidification temperature of the system, but is preferably −10° C. or higher, more preferably −1° C. or higher. Specifically, the endpoint temperature of the crystal precipitation step may be set within the range of −10 to 35° C., preferably −1 to 30° C., more preferably −1 to 25° C.

In the crystal precipitation step, it is preferred to control the precipitation amount of crystal per unit time and thereby control the formation of supersaturation. A preferred precipitation amount per unit time is, for example, equal to or less than a rate to precipitate about 50% of the total precipitation amount per unit time (that is, at most 50% amount/hour), and preferably equal to or less than a rate to precipitate 25% of the total precipitation amount per unit time (that is, at most 25% amount/hour).

In one or more embodiments of the present invention, the crystal precipitation step includes lowering the temperature of the mixed liquid over time, that is, a cooling crystallization step. In cooling crystallization, the mixed liquid is cooled to lower the solubility in the liquid phase of each component such as reduced coenzyme Q10 and to promote crystallization. The step of cooling the mixed liquid is preferably performed successively after the above step of maintaining the mixed liquid at a temperature of 25° C. or higher for a constant time to cause precipitation of the crystal capable of precipitation at this temperature. Lowering the temperature of the mixed liquid over time includes continuously lowering the temperature of the mixed liquid with elapsed time, stepwise lowering the temperature of the mixed liquid, and combinations thereof. When lowering the temperature of the mixed liquid over time, the cooling rate is not particularly limited, but, for example, the cooling rate is such that the temperature drop per hour is 30° C. or lower, preferably 25° C. or lower, more preferably 20° C. or lower, more preferably 15° C. or lower, more preferably 10° C. or lower, more preferably 0.5° C. or higher, and more preferably 1° C. or higher. The cooling rate in the case of lowering the temperature of the mixed liquid over time may be a constant, or may be subject to change. In particular, if the cooling rate is such that, as the temperature of the mixed liquid decreases, the cooling rate increases continuously or stepwise, that is, so that the magnitude of the temperature decrease per hour becomes larger, efficient crystallization of reduced coenzyme Q10, whose residual amount in the liquid phase decreases as the temperature of the mixed liquid decreases, can be carried out. For example, until the temperature of the mixed liquid reaches 25° C., cool the mixed liquid at a rate at which the temperature decrease per hour is preferably 5° C. or lower, more preferably 3° C. or lower, and, in a stage in which the mixed liquid is further cooled to a temperature of less than 25° C., it is possible to cool the mixed liquid at a rate at which the temperature decrease per hour is preferably 6° C. or higher, more preferably 8° C. or higher. In one or more embodiments of the present invention, the endpoint temperature that is reached by lowering the temperature of the mixed liquid over time is preferably 25° C. or lower, more preferably 20° C. or lower, more preferably 10° C. or lower, more preferably 7° C. or lower, and more preferably 5° C. or lower. The lower limit of the endpoint temperature is the solidification temperature of the system of the mixed liquid, and is preferably −10° C. or higher, preferably −7° C. or higher, preferably −5° C. or higher, and more preferably −1° C. or higher. For example, the endpoint temperature may be within the range of −10 to 25° C., preferably −10 to 20° C., more preferably −7 to 10° C., still more preferably −5 to 7° C., and particularly preferably −5 to 5° C.

It is preferred to carry out the precipitation of the crystal while causing forced flow of the mixed liquid after addition of the seed crystal. In order to suppress the formation of supersaturation and to carry out nucleation and crystal growth smoothly, or from the viewpoint of higher quality, as the agitation power requirement per unit volume, it is good to impart the mixed liquid a flowing of typically about 0.005 kW/m³ or more, preferably 0.01 kW/m³ or more, more preferably 0.015 kW/m³ or more, and still more preferably 0.03 kW/m³ or more. The above forced flow is typically imparted by rotation of a stirring blade, but if the above flowing is obtained, it is not necessarily required to use a stirring blade, and, for example, a method using circulation of a mixed liquid or the like may be utilized.

In the production method of one or more embodiments of the present invention, the step of separating, from the solution, a composition in solid form containing reduced coenzyme Q10, reduced demethoxy Q10, and reduced coenzyme Q9 is not particularly limited, and, in addition to the above cooling crystallization, methods such as poor solvent crystallization and concentration crystallization can be utilized. It is preferred that it be cooling crystallization, or a method combining cooling crystallization with another crystallization method. Poor solvent crystallization is a method that, by mixing a poor solvent with the mixed liquid, lowers the solubility and causes crystallization of reduced coenzyme Q10. Here, the term poor solvent refers to a solvent in which either dissolution of reduced coenzyme Q10 hardly occurs or dissolution of reduced coenzyme Q10 does not occur at all. It is preferred that the poor solvent mutually dissolve with an organic solvent used in a solution that contains reduced coenzyme Q10 and the like.

As methods to mix a poor solvent with a solution, one may add the poor solvent to the solution, or one may add the solution to the poor solvent. Examples of other crystallization methods to be combined with cooling crystallization include, in addition to the above-described poor solvent crystallization, for example, concentration crystallization that causes precipitation of the crystal by concentrating a solution, and the like.

The crystal or crystalline solid of reduced coenzyme Q10 obtained by the above method can be recovered, for example, by a conventionally known method, through the steps of solid-liquid separation and drying. For example, for solid-liquid separation, the use of pressure filtration, filtration by centrifugation, and the like is possible. Additionally, the crystal or the crystalline solid after drying can also be subjected to pulverization and classification as necessary, and recovered.

As one of the more preferred aspects, by performing, under heating, the drying of the crystal or crystalline solid of reduced coenzyme Q10 after the above solid-liquid separation, it is possible to improve the content ratio of the reduced coenzyme Q10 crystal. For this purpose, the drying temperature is preferably 33° C. or higher, more preferably 34° C. or higher, and still more preferably 35° C. or higher. The upper limit is typically 46° C. or lower, preferably 45° C. or lower. For example, the drying temperature can be set within the range of 33 to 46° C., preferably 34 to 46° C., and more preferably within the range of 35 to 45° C. When below 33° C., drying proceeds, but the content ratio of the reduced coenzyme Q10 crystal hardly increases. In addition, when exceeding 46° C., during drying the reduced coenzyme Q10 crystal may undergo melting. Also, the heating time in the case of performing drying under the above temperature conditions is not particularly limited, but is, preferably 4 hours or more, preferably 10 hours or more, and more preferably 20 hours or more.

Note that, in the crystal precipitation step, when the content ratio of the reduced coenzyme Q10 crystal for the purpose has already been achieved, it is not limited to the above, and, for example, the drying may be carried out at 20° C. or higher, preferably 25° C. or higher, more preferably 30° C. or higher.

In addition, in the production method of one or more embodiments of the present invention, it is preferred that each step, specifically the mixed liquid formation step, the crystal precipitation step, recovery steps such as solid-liquid separation and drying, described above, and other subsequent processing steps and the like, be carried out under a deoxygenated atmosphere. A deoxygenated atmosphere can be achieved by substitution of the atmosphere with an inert gas, by reduced pressure, by boiling, or by combining these. At least, substitution of the atmosphere with an inert gas, i.e., using an inert gas atmosphere, is preferred. Examples of the above inert gas include nitrogen gas, helium gas, argon gas, hydrogen gas, carbon dioxide gas, and the like, and nitrogen gas is preferred.

Whether the reduced coenzyme Q10 crystal of the Form I type or Form II type are contained in the obtained reduced coenzyme Q10 crystal or crystalline solid, and the content ratio thereof, can be determined, for example, by measuring with a differential scanning calorimeter (DSC).

The Form II type reduced coenzyme Q10 crystal, when measured by DSC at a heating rate of 1° C./min, shows an endothermic peak in the vicinity of 52±2° C., and the Form I type reduced coenzyme Q10 crystal, under the same conditions, shows an endothermic peak in the vicinity of 48±1° C. Even when a Form II type reduced coenzyme Q10 crystal is in a state mixed with a conventional Form I type reduced coenzyme Q10 crystal or its crystalline solid, it is possible to make a determination of the presence or absence of the Form II type reduced coenzyme Q10 crystal and its content ratio by the presence or absence of the peak around 52±2° C., the height of that endothermic peak, and the ratio of the heats of absorption.

When producing a composition in solid form through a step of separation of reduced coenzyme Q10, reduced demethoxy Q10, and reduced coenzyme Q9 from a solution, for example, in accordance with each form such as powder, granules, tablets, hard capsules, or the like, it is possible to produce the composition by conventionally known steps.

For example, one can produce a reduced coenzyme Q10 composition in powder form by mixing, as powder raw materials, reduced coenzyme Q10 and emulsified starch and, if necessary, substances other than the reduced coenzyme Q10 and the emulsified starch such as antioxidants, emulsifiers, binders, and excipients.

Further, one can produce a reduced coenzyme Q10 composition in granule form, for example, by mixing, as powder raw materials, reduced coenzyme Q10 and emulsified starch, a binder, and, as necessary, substances other than the reduced coenzyme Q10 and the emulsified starch such as an antioxidant and an emulsifier, adding water to the mixture, and going through steps of granulation, drying, classification, and pulverization.

Furthermore, one can produce a reduced coenzyme Q10 composition in tablet form by tableting the reduced coenzyme Q10 composition in the powder or granule form by a conventional method.

One can produce a reduced coenzyme Q10 composition in hard capsule form by filling a hard capsule with the reduced coenzyme Q10 composition in the powder or granule form by a conventional method.

The granulation method is not particularly limited, and, for example, may be appropriately selected from methods such as an extrusion granulation method, a stirring granulation method, a tumbling granulation method (a rotating granulation method), a dry granulation method, a compression granulation method, a powder bonding method, a fluidized bed granulation method, a coacervation method, a spray-drying method, a cold spray method, an evaporation method, and an in-liquid curing method. The granulation method is preferably carried out by a granulation method in which pressure is applied (for example, the extrusion granulation method, the stirring granulation method, the tumbling granulation method, the dry granulation method, the compression granulation method, the powder bonding method, and the like) or by the fluidized bed granulation method, more preferably by the extrusion granulation method, the stirring granulation method, or the fluidized bed granulation method, and particularly preferably by the extrusion granulation method or the stirring granulation method.

In the present embodiment, when the composition contains the reduced coenzyme Q10 crystal, in the granulation process the reduced coenzyme Q10 crystal is not completely dissolved or melted, and is mixed and granulated while basically maintaining the crystal state.

The components other than the liquid binder of the mixture to be subjected to granulation may be in the form of a powder mixture. The characteristics of the respective components other than the liquid binder of the mixture are as described above.

Examples of the liquid binder include water, ethanol, and the like. Additionally, the above-described binder may be caused to undergo dissolution in a liquid such as water or ethanol and used as a liquid binder. As the liquid binder, water is particularly preferred.

Granulation can be carried out by appropriately setting conditions so that a composition in solid form having dimensions according to the application for the purpose is obtained.

It is preferred to further include a drying step of drying, after granulation, the reduced coenzyme Q10 composition in solid form and removing, by drying, volatile components derived from each raw material component and the liquid binder. Additionally, as necessary, by sieving or the like, it is possible to perform separation and recovery of a composition in solid form having a desired particle size.

One can produce the reduced coenzyme Q10 composition in tablet form by tableting a composition in powder form, or a composition in granule form obtained through steps such as granulation and drying, but the tableting method is not particularly limited. For example, it is possible to appropriately select from methods such as the direct compression method, the semi-direct compression method, a dry granulation compression method, and a wet granulation compression method. The tableting method is preferably performed by the direct compression method or the semi-direct compression method, and the direct compression method is more preferred.

The reduced coenzyme Q10 composition according to the present embodiment can also be added to general food and beverage. The food and beverage to which the reduced coenzyme Q10 composition is added is not particularly limited, and, for example, the reduced coenzyme Q10 composition can be appropriately added to and used in beverages such as milk beverages, soft drinks, sports drinks, nutritional drinks, beauty drinks, liquid nutritional preparations, and the like, confectionery such as chewing gum, chocolate, candies, jelly, cakes, biscuits, crackers, and the like, frozen desserts such as ice cream, ice confections, and the like, noodles such as udon, Chinese noodles, spaghetti, instant noodles, and the like, fish paste products such as kamaboko, chikuwa, hanpen, and the like, seasonings such as dressings, mayonnaise, sauces, and the like, bread, ham, rice porridge, cooked rice, soups, various retort foods, various frozen foods, and the like. A food or beverage containing a reduced coenzyme Q10 composition according to the present embodiment can be used in applications such as so-called health food, supplement, functional food, Food with Function Claims, nutritional supplement, Food for Specified Health Uses, Nutrient Function Food, nursing care food, Smile Care Food, mastication and swallowing aid food, thick flowing food, and food for patients and the like. Alternatively, it can also be used as other food forms. Furthermore, the reduced coenzyme Q10 composition according to one or more embodiments of the present invention can also be added to general pharmaceuticals. Furthermore, it can also be used for pet food, livestock feed, and the like.

### <<Second Aspect (Disclosure of Japanese Patent Application No. 2025-058008)>>

The second aspect of one or more embodiments of the present invention relates to a method that improves the polymorphic stability of the reduced coenzyme Q10 crystal, and to a method for storing the reduced coenzyme Q10 crystal.

In the case of a crystal form other than the stable form, for example, during operations such as crystallization, drying, pulverization, or the like, or during storage, there is a possibility that a transition to the stable form may occur. Although this is a very natural phenomenon in which a substance attempts to undergo a change to an energetically stable state, such a transition can result in changes in the physical properties of the crystal, there is a possibility that quality variations occur in the crystal and in a composition in which the crystal is an active ingredient. In the product such as pharmaceutical or functional food or the like, crystal polymorphism should be strictly controlled, and therefore, it is desirable to prevent a transition of the reduced coenzyme Q10 crystal to the other crystal form, that is, to improve polymorphic stability of the reduced coenzyme Q10 crystal.

Accordingly, the second aspect of one or more embodiments of the present invention aims to provide a method for improving the polymorphic stability of a reduced coenzyme Q10 crystal, and a method for storing a reduced coenzyme Q10 crystal.

The present inventors have found that, by allowing coexistence of a reduced coenzyme Q10 crystal and analogs having a chemical structure similar to reduced coenzyme Q10 in a composition, and by controlling the peak area ratio in a chromatogram among peaks corresponding to the multiple kinds of analogs so as to be within a specific range, transition of the reduced coenzyme Q10 crystal to the other crystal form becomes less likely to occur, that is, the polymorphic stability of the reduced coenzyme Q10 crystal improves, and have thus completed the second aspect of one or more embodiments of the present invention.

The gist of the second aspect of one or more embodiments of the present invention is as follows.
(1) A method for improving a polymorphic stability of a reduced coenzyme Q10 crystal comprising
   a step of causing coexistence, in a composition, of the reduced coenzyme Q10 crystal with reduced demethoxy Q10 and reduced coenzyme Q9, wherein, in a chromatogram of the composition obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 mL/min, and
   a detection wavelength: 190 nm,
an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more.
(2) The method according to the above (1),
   wherein, in the chromatogram of the composition, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 1.0 or more.
(3) The method according to the above (1),
   wherein, in the chromatogram of the composition, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 2.0 or more.
(4) The method according to any one of the above (1) to (3),
   wherein the reduced coenzyme Q10 crystal is a Form I crystal.
(5) The method according to any one of the above (1) to (4),
   wherein the composition is in solid form.
(6) The method according to any one of the above (1) to (5),
   wherein the composition further contains an antioxidant.
(7) A method for storing a reduced coenzyme Q10 crystal comprising
   a step of storing the reduced coenzyme Q10 crystal while causing coexistence of the reduced coenzyme Q10 crystal with reduced demethoxy Q10 and reduced coenzyme Q9 in a composition, wherein, in a chromatogram of the composition obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 mL/min, and
   a detection wavelength: 190 nm,
an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more.
(8) The method according to the above (7),
   wherein, in the chromatogram of the composition, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 1.0 or more.
(9) The method according to the above (7),
   wherein, in the chromatogram of the composition, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 2.0 or more.
(10) The method according to any one of the above (7) to (9),
   wherein the reduced coenzyme Q10 crystal is a Form I crystal.
(11) The method according to any one of the above (7) to (10),
   wherein the composition is in solid form.
(12) The method according to any one of the above (7) to (11),
   wherein the composition further contains an antioxidant.

According to the second aspect of one or more embodiments of the present invention, there are provided a method for improving the polymorphic stability of a reduced coenzyme Q10 crystal, and a storage method for a reduced coenzyme Q10 crystal in which a polymorphic transition of the reduced coenzyme Q10 crystal is unlikely to occur.

### I a Method for Improving the Polymorphic Stability of a Reduced Coenzyme Q10 Crystal

According to one embodiment, a method for improving polymorphic stability of a reduced coenzyme Q10 crystal includes a step of causing coexistence of the reduced coenzyme Q10 crystal with reduced demethoxy Q10 and reduced coenzyme Q9 in a composition.

In the present specification, “coexistence” means that two or more substances are present simultaneously in the same system. Therefore, “to cause coexistence of a reduced coenzyme Q10 crystal with reduced demethoxy Q10 and reduced coenzyme Q9 in a composition” means that there is present, simultaneously in a composition, a reduced coenzyme Q10 crystal, reduced demethoxy Q10, and reduced coenzyme Q9. The obtained composition contains reduced coenzyme Q10 crystal, reduced demethoxy Q10, and reduced coenzyme Q9. A composition that contains reduced coenzyme Q10 crystal, reduced demethoxy Q10, and reduced coenzyme Q9 is hereinafter, as appropriate, referred to as “a composition containing reduced coenzyme Q10 crystal, and the like” or simply “composition.”

### <Coenzyme Q10>

The reduced coenzyme Q10 has the following chemical structure. The composition may contain oxidized coenzyme Q10 having the following chemical structure. In one embodiment, a main component of coenzyme Q10 contained in the composition is reduced coenzyme Q10. Here, the main component means that, with respect to the total weight of coenzyme Q10 in the composition, the content of reduced coenzyme Q10 is, for example, 50 wt % or more, typically 60 wt % or more, preferably 70 wt % or more, more preferably 80 wt % or more, still more preferably 90 wt % or more, particularly preferably 95 wt % or more, and especially 98 wt % or more.

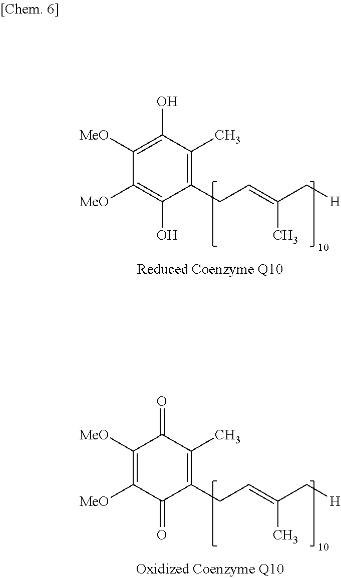

Reduced coenzyme Q10 can be obtained, for example, by known methods such as synthesis, fermentation, and extraction from a natural product, as well as by combining those with a reduction reaction as necessary. Preferably, it can be obtained by reducing oxidized coenzyme Q10, such as existing high-purity coenzyme Q10, or a mixture of oxidized coenzyme Q10 and reduced coenzyme Q10, using a general reducing agent, for example, sodium dithionite, sodium borohydride, ascorbic acids and the like.

The composition may further contain reduced coenzyme Q10 in the state of amorphous. The reduced coenzyme Q10 crystal in the composition may contain amorphous components therein. An amount of the reduced coenzyme Q10 crystal with respect to the total weight of reduced coenzyme Q10 in the composition is preferably 80 wt % or more, more preferably 90 wt % or more, even more preferably 95 wt % or more, and most preferably 98 wt % or more. Note that the degree of crystallization of each component such as reduced coenzyme Q10 and the like contained in the composition can be calculated, in accordance with the following formula, from the theoretical melting heat obtained from the content of each component in the composition and from the measured melting heat data obtained by measuring the crystal melting heat by a differential scanning calorimeter (DSC) analysis.

$$Degree of crystallization ( \% ) = ( measured melting enthalpy / theoretical melting enthalpy ) \times 100$$

In reduced coenzyme Q10, there are two crystal polymorphs, the Form I type and the Form II type. Specifically, the crystal form of reduced coenzyme Q10 having a melting point about 48° C. and showing characteristic peaks at diffraction angles (2θ±0.2°) of 3.1°, 18.7°, 19.0°, 20.2°, and 23.0° in powder X-ray (Cu-Kα) diffraction is a Form I crystal, and the crystal form of reduced coenzyme Q10 having a melting point about 52° C. and showing characteristic peaks at diffraction angles (2θ±0.2°) of 11.5°, 18.2°, 19.3°, 22.3°, 23.0°, and 33.3° in powder X-ray (Cu-Kα) diffraction is a Form II crystal.

The composition can contain, as reduced coenzyme Q10 (QH) crystal, Form I crystal and Form II crystal at any blending proportion. For example, with respect to the total weight of Form I crystal and Form II crystal, the content of Form I crystal can be preferably 90 wt % or more, more preferably 95 wt % or more, particularly preferably 98 wt % or more, and preferably 100 wt % or less. For example, with respect to the total weight of the Form I crystal and the Form II crystal, the content of the Form I crystal can be preferably 90 to 100 wt %, more preferably 95 to 100 wt %, particularly preferably 98 to 100 wt %. Because a transition of the reduced coenzyme Q10 crystal in the composition to the other crystal form is unlikely to occur, the composition can maintain over a long period the properties and quality of the composition at the initial time of manufacture.

In one embodiment, the content of reduced coenzyme Q10 in the composition is not particularly limited, but is, with respect to the total weight of the composition, typically 0.1 wt % or more, preferably 0.5 wt % or more, more preferably 1 wt % or more, particularly preferably 2 wt % or more, and still more preferably 5 wt % or more, and 50 wt % or less, preferably 49 wt % or less, more preferably 48 wt % or less, particularly preferably 47 wt % or less, and further preferably 46 wt % or less. That is, the content of reduced coenzyme Q10 in the composition can be, with respect to the total weight of the composition, within the range of 0.1 to 50 wt %, preferably 0.5 to 49 wt %, more preferably 1 to 48 wt %, particularly preferably 2 to 47 wt %, and still more preferably 5 to 46 wt %.

In another embodiment, the content of reduced coenzyme Q10 in the composition is not particularly limited, but is, with respect to the total weight of the composition, typically 51 wt % or more, preferably 56 wt % or more, more preferably 61 wt % or more, particularly preferably 66 wt % or more, and still more preferably 71 wt % or more, and 99 wt % or less, preferably 98 wt % or less, more preferably 97 wt % or less, particularly preferably 96 wt % or less, and even more preferably 95 wt % or less. That is, the content of reduced coenzyme Q10 in the composition can be, with respect to the total weight of the composition, within the range of 51 to 99 wt %, preferably 56 to 98 wt %, more preferably 61 to 97 wt %, particularly preferably 66 to 96 wt %, and still more preferably 71 to 95 wt %.

Reduced demethoxy Q10 (which may hereinafter be referred to as “RD”) and reduced coenzyme Q9 (which may hereinafter be referred to as “RQ9”) have the following chemical structure, respectively.

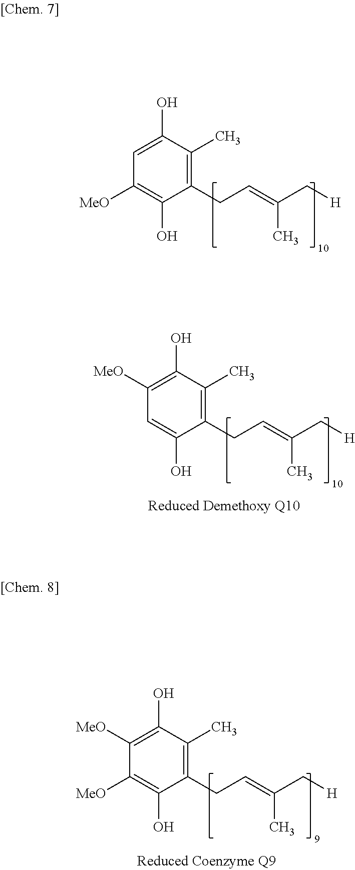

Reduced demethoxy Q10 (RD) corresponds to a compound in which the methoxy group in QH is missing. Reduced coenzyme Q9 (RQ9) corresponds to a compound in which the isoprene chain in QH is shorter by one unit. Note that, in the present specification, “reduced demethoxy Q10 (RD)” also includes analogs of reduced demethoxy Q10. Examples of the analogs of the reduced demethoxy Q10 include compounds in which the isoprene chain of the reduced demethoxy Q10 has undergone cyclization at one site or double-bond formation at one site, compounds in which cyclization has occurred at one site or at two or more sites of the isoprene chain of QH, and compounds in which the formation of a double bond has occurred at one site or at two or more sites of the isoprene chain of QH. RD and RQ9, as with QH, can be obtained by known methods such as synthesis, fermentation, or extraction from natural products and as necessary, that combined with a reduction reaction. Preferably, RD and RQ9, that are generated together as by-products when reduced coenzyme Q10 is produced by reducing a composition containing existing oxidized coenzyme Q10 with a general reducing agent, for example, sodium dithionite, sodium borohydride, ascorbic acids, or the like, may be used as they are.

RD and RQ9 in the composition may be in the crystal state, in the amorphous state, or the mixture of crystalline state and amorphous state. Amorphous components may be contained in the crystalline RD and RQ9. RD and RQ9 may contain, with respect to the total weight of RD and RQ9, preferably 80 wt % or more, more preferably 90 wt % or more, still more preferably 95 wt % or more, and most preferably 98 wt % or more, of crystal.

The total content of RD and RQ9 in the composition is not particularly limited, but is, with respect to the total weight of the composition, typically 0.001 wt % or more, preferably 0.005 wt % or more, more preferably 0.01 wt % or more, particularly preferably 0.015 wt % or more, and further preferably 0.02 wt % or more, and 10 wt % or less, preferably 8 wt % or less, more preferably 6 wt % or less, particularly preferably 4 wt % or less, and still more preferably 2 wt % or less. That is, the total content of RD and RQ9 can be, with respect to the total weight of the composition, typically within the range of 0.001 to 10 wt %, preferably 0.005 to 8 wt %, more preferably 0.01 to 6 wt %, particularly preferably 0.015 to 4 wt %, and further preferably 0.02 to 2 wt %. The content of RD in the composition is, with respect to the total weight of the composition, within the range of preferably 0.0005 to 5 wt %, more preferably 0.001 to 3 wt %, and the content of RQ9 in the composition is, with respect to the total weight of the composition, within the range of preferably 0.002 to 4 wt %, more preferably 0.005 to 2 wt %.

The content of RD in the composition is, with respect to the total weight of the composition, for example, 0.0005 wt % or more, in one embodiment, 0.001 wt % or more and, for example, 5 wt % or less, in one embodiment, 3 wt % or less, and, for example, within the range of 0.0005 wt % to 5 wt %, in one embodiment, 0.001 wt % to 3 wt %, and the content of RQ9 in the composition is, with respect to the total weight of the composition, for example, 0.002 wt % or more, in one embodiment, 0.005 wt % or more, and, for example, 4 wt % or less, in one embodiment, 2 wt % or less, and, for example, within the range of 0.002 wt % to 4 wt %, in one embodiment, 0.005 wt % to 2 wt %.

The present inventors have found that, by adjusting the blending ratio of RD and RQ9 so that a peak area ratio in the chromatogram of RD and RQ9 in the composition is within a specific range, although the reason is not clear, a polymorphic transition of the reduced coenzyme Q10 (QH) crystal is unlikely to occur (that is, the polymorphic stability of QH crystal improves). Specifically, in a chromatogram of a composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more. The above peak area ratio (RD/RQ9) may be 0.5 or more, 0.8 or more, 1.0 or more, 1.5 or more, or 2.0 or more and may be 10.0 or less, 8.0 or less, 6.0 or less, 4.0 or less, or 3.0 or less. The above peak area ratio (RD/RQ9) may be within the range of 0.3 to 10.0, 0.5 to 10.0, 0.5 to 3.0, 0.8 to 8.0, 1.0 to 6.0, 1.0 to 2.0, 1.5 to 4.0, or 2.0 to 3.0.

In another embodiment, specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more. The above peak area ratio (RD/RQ9) may be, for example, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, or in one embodiment, 2.0 or more. The upper limit of the above peak area ratio (RD/RQ9) is not particularly limited, but, it may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.0 or less, in one embodiment, 2.8 or less, in one embodiment, 2.6 or less, in one embodiment, 2.4 or less, in one embodiment, 2.2 or less, or in one embodiment, 2.1 or less. That is, the above peak area ratio may be 0.3 or more, and, for example, may be within the range of 0.3 to 10.0, in one embodiment, 0.3 to 9.0, in one embodiment, 0.3 to 8.0, in one embodiment, 0.3 to 7.0, in one embodiment, 0.3 to 6.0, in one embodiment, 0.3 to 5.0, in one embodiment, 0.3 to 4.0, in one embodiment, 0.3 to 3.0, in one embodiment, 0.3 to 2.8, in one embodiment, 0.3 to 2.6, in one embodiment, 0.3 to 2.4, in one embodiment, 0.3 to 2.2, in one embodiment, 0.3 to 2.1, in one embodiment, 0.4 to 2.1, in one embodiment, 0.5 to 2.1, in one embodiment, 0.6 to 2.1, in one embodiment, 0.7 to 2.1, in one embodiment, 0.8 to 2.1, in one embodiment, 0.9 to 2.1, in one embodiment, 1.0 to 2.1, in one embodiment, 1.1 to 2.1, in one embodiment, 1.2 to 2.1, in one embodiment, 1.3 to 2.1, in one embodiment, 1.4 to 2.1, in one embodiment, 1.5 to 2.1, in one embodiment, 1.6 to 2.1, in one embodiment, 1.7 to 2.1, in one embodiment, 1.8 to 2.1, in one embodiment, 1.9 to 2.1, in one embodiment, 2.0 to 2.1, in one embodiment, 0.5 to 10.0, in one embodiment, 0.5 to 3.0, in one embodiment, 0.8 to 8.0, in one embodiment, 1.0 to 6.0, in one embodiment, 1.0 to 2.0, in one embodiment, 1.5 to 4.0, or in one embodiment, 2.0 to 3.0.

Here, the peak area of each compound in the chromatogram can be calculated by an automatic integration method (in which an integrator automatically calculates from the signal intensity of the detector). In addition, the identification of each peak is performed by mass spectrometry. The peak area of each compound in the chromatogram can be appropriately changed by adjusting the content of each compound in the composition.

In the chromatogram obtained by high performance liquid chromatography under the above conditions, the area of the peak containing each of reduced coenzyme Q9 (RQ9), reduced demethoxy Q10 (RD), reduced coenzyme Q10 (QH), decaprenylphenol and reduced coenzyme Q11 (RQ11) to be described later, and oxidized coenzyme Q10 (QX) refers to the area of the peak detected at the retention time (RT) shown in the table below, or at the relative retention time (RRT) based on the retention time (RT, 14.72 min in Table 2) of the peak top of QH.

TABLE 2

| | Reduced | Reduced | Reduced |
|---|---|---|---|
| | Coenzyme Q9 | Demethoxy Q10 | Coenzyme Q10 |
| | (RQ9) | (RD) | (QH) |
| RT(min) | 10.10 to 10.75 | 12.90 to 13.90 | 14.00 to 15.75 |
| RRT | 0.69 to 0.73 | 0.88 to 0.94 | 0.95 to 1.07 |

| | | Reduced | Oxidized |
|---|---|---|---|
| | | Coenzyme Q11 | Coenzyme Q10 |
| | Decaprenylphenol | (RQ11) | (QX) |
| RT(min) | 19.75 to 20.7 | 21.30 to 22.0 | 24.75 to 26.25 |
| RRT | 1.34 to 1.41 | 1.45 to 1.49 | 1.68 to 1.78 |

In the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but, for example, may be 0.0005 or more, in one embodiment, 0.001 or more, in one embodiment, 0.002 or more, in one embodiment, 0.003 or more, in one embodiment, 0.004 or more, in one embodiment, 0.005 or more, and in one embodiment, 0.01 or less, in one embodiment, 0.008 or less, in one embodiment, 0.006 or less.

In the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but is within the range of preferably 0.0005 to 0.01, more preferably 0.001 to 0.008, still more preferably 0.002 to 0.008, and particularly preferably 0.004 to 0.006.

In the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but, for example, is within the range of 0.0005 to 0.01, in one embodiment, 0.001 to 0.008, in one embodiment, 0.001 to 0.006, in one embodiment, 0.002 to 0.006, in one embodiment, 0.003 to 0.006, in one embodiment, 0.004 to 0.006, in one embodiment, 0.005 to 0.006, and in one embodiment 0.002 to 0.008.

In the chromatogram obtained by high performance liquid chromatography under the above conditions, the area ratio (RQ9/QH) of the peak containing reduced coenzyme Q9 (RQ9) to the peak containing reduced coenzyme Q10 (QH) is not particularly limited, but, for example, may be 0.001 or more, in one embodiment, may be 0.002 or more, in one embodiment, may be 0.01 or less, in one embodiment, may be 0.008 or less, in one embodiment, may be 0.007 or less, in one embodiment, may be 0.006 or less, in one embodiment, may be 0.005 or less, in one embodiment, may be 0.004 or less, and in one embodiment may be 0.003 or less.

In the chromatogram obtained by high performance liquid chromatography under the above conditions, the area ratio (RQ9/QH) of the peak containing reduced coenzyme Q9 (RQ9) to the peak containing reduced coenzyme Q10 (QH) is not particularly limited, but is within the range of preferably 0.001 to 0.01, more preferably 0.001 to 0.006, still more preferably 0.001 to 0.005, and particularly preferably 0.002 to 0.005.

In the chromatogram obtained by high performance liquid chromatography under the above conditions, the area ratio (RQ9/QH) of the peak containing reduced coenzyme Q9 (RQ9) to the peak containing reduced coenzyme Q10 (QH) is not particularly limited, but, for example, is within the range of 0.001 to 0.01, in one embodiment, 0.001 to 0.008, in one embodiment, 0.001 to 0.007, in one embodiment, 0.001 to 0.006, in one embodiment, 0.001 to 0.005, in one embodiment, 0.001 to 0.004, in one embodiment, 0.001 to 0.003, in one embodiment, 0.002 to 0.003, in one embodiment, 0.002 to 0.005.

In the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but, for example, may be 0.001 or more, in one embodiment, may be 0.005 or more, in one embodiment, may be 0.007 or more, in one embodiment, may be 0.008 or more, in one embodiment, may be 0.009 or more, in one embodiment, may be 0.01 or more, and in one embodiment may be 0.03 or less, in one embodiment, may be 0.02 or less, in one embodiment, may be 0.015 or less, in one embodiment, may be 0.012 or less, and in one embodiment may be 0.01 or less.

In the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but is within the range of preferably 0.001 to 0.03, more preferably 0.005 to 0.02, still more preferably 0.007 to 0.015, and particularly preferably 0.007 to 0.01.

In the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but, for example, is within the range of 0.001 to 0.03, in one embodiment, 0.005 to 0.03, in one embodiment, 0.007 to 0.03, in one embodiment, 0.007 to 0.02, in one embodiment, 0.007 to 0.015, in one embodiment, 0.007 to 0.012, in one embodiment, 0.008 to 0.012, in one embodiment, 0.009 to 0.012, in one embodiment, 0.01 to 0.012, in one embodiment, 0.005 to 0.02, in one embodiment, 0.007 to 0.01, in one embodiment, 0.008 to 0.01, and in one embodiment, 0.009 to 0.01.

The composition, in addition to the above RD and RQ9, may contain, as necessary, a QH analog. Examples of the QH analogs, including decaprenylphenol and reduced coenzyme Q11 (hereinafter may be referred to as “RQ11”) as shown below, can be given.

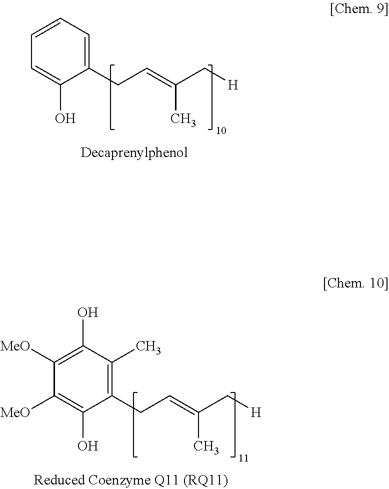

QH analogs such as the above decaprenylphenol and RQ11 can, similarly to QH, be obtained by known methods such as synthesis, fermentation, or extraction from natural products and as necessary, that combined with a reduction reaction. Preferably, QH analogs, such as decaprenylphenol and RQ11, that are generated together as by-products when reduced coenzyme Q10 is produced by reducing a composition containing existing oxidized coenzyme Q10 with a general reducing agent, for example, sodium dithionite, sodium borohydride, ascorbic acids, or the like, may be used as they are.

The total content of QH analog (RD, RQ11, RQ9 and decaprenylphenol) in the composition is not particularly limited, but is, with respect to the total weight of the composition, typically 8 wt % or less, preferably 7 wt % or less, more preferably 6 wt % or less, particularly preferably 5 wt % or less, still more preferably 4 wt % or less, and is typically 0.006 wt % or more, preferably 0.008 wt % or more, more preferably 0.01 wt % or more. The total content of QH analogs (RD, RQ11, RQ9, and decaprenylphenol) in the composition is typically 0.006 to 8 wt %, preferably 0.006 to 7 wt %, more preferably 0.008 to 6 wt %, still more preferably 0.008 to 5 wt %, and particularly preferably 0.01 to 4 wt %.

The content of decaprenylphenol in the composition is, with respect to the total weight of the composition, in one embodiment, 0.001 wt % or more, in one embodiment, 0.002 wt % or more, in one embodiment, 3 wt % or less, in one embodiment, 2 wt % or less, and the content of RQ11 is, with respect to the total weight of the composition, in one embodiment, 0.005 wt % or more, in one embodiment, 0.01 wt % or more, preferably 5 wt % or less, and more preferably 3 wt % or less.

The content of decaprenylphenol in the composition is, with respect to the total weight of the composition, preferably within the range of 0.001 to 3 wt %, more preferably 0.002 to 2 wt %. The content of RQ11 in the composition is, with respect to the total weight of the composition, preferably within the range of 0.005 to 5 wt %, more preferably 0.01 to 3 wt %.

In the chromatogram obtained by high performance liquid chromatography under the above conditions, the area ratio (decaprenylphenol/QH) of the peak containing decaprenylphenol to the peak containing QH is not particularly limited, but, for example, may be 0.001 or more, in one embodiment, 0.002 or more, in one embodiment, 0.003 or more, in one embodiment, 0.004 or more, in one embodiment, 0.005 or more, in one embodiment, 0.006 or more, in one embodiment, 0.007 or more, in one embodiment, 0.008 or more, in one embodiment, 0.009 or more, in one embodiment, 0.05 or less, in one embodiment, 0.03 or less, and in one embodiment, 0.01 or less.

In the chromatogram obtained by high performance liquid chromatography under the above conditions, the area ratio (decaprenylphenol/QH) of the peak containing decaprenylphenol to the peak containing QH is not particularly limited, but is preferably within the range of 0.001 to 0.05, more preferably 0.003 to 0.03, and still more preferably 0.005 to 0.01.

In the chromatogram obtained by high performance liquid chromatography under the above conditions, the area ratio (decaprenylphenol/QH) of the peak containing decaprenylphenol to the peak containing QH is not particularly limited, but is, for example, within the range of 0.001 to 0.05, in one embodiment, 0.001 to 0.03, in one embodiment, 0.001 to 0.01, in one embodiment, 0.002 to 0.01, in one embodiment, 0.003 to 0.01, in one embodiment, 0.004 to 0.01, in one embodiment, 0.005 to 0.01, in one embodiment, 0.006 to 0.01, in one embodiment, 0.007 to 0.01, in one embodiment, 0.008 to 0.01, in one embodiment, 0.009 to 0.01, and in one embodiment, 0.003 to 0.03.

In the chromatogram obtained by high performance liquid chromatography under the above conditions, the area ratio (RQ11/QH) of the peak containing RQ11 to the peak containing QH is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.01 or more, in one embodiment, 0.015 or more, in one embodiment, 0.016 or more, in one embodiment, 0.017 or more, in one embodiment, 0.018 or more, in one embodiment, 0.019 or more, in one embodiment, 0.02 or more, in one embodiment, 0.05 or less, in one embodiment, 0.04 or less, in one embodiment, 0.03 or less, in one embodiment, 0.025 or less, and in one embodiment, 0.02 or less.

In the chromatogram obtained by high performance liquid chromatography under the above conditions, the area ratio (RQ11/QH) of the peak containing RQ11 to the peak containing QH is not particularly limited, but is preferably within the range of 0.001 to 0.05, more preferably 0.005 to 0.04, and even more preferably 0.01 to 0.03.

In the chromatogram obtained by high performance liquid chromatography under the above conditions, the area ratio (RQ11/QH) of the peak containing RQ11 to the peak containing QH is not particularly limited, but, for example, is within the range of 0.001 to 0.05, in one embodiment, 0.005 to 0.05, in one embodiment, 0.006 to 0.05, in one embodiment, 0.006 to 0.04, in one embodiment, 0.006 to 0.03, in one embodiment, 0.006 to 0.025, in one embodiment, 0.007 to 0.025, in one embodiment, 0.008 to 0.025, in one embodiment, 0.009 to 0.025, in one embodiment, 0.01 to 0.025, in one embodiment, 0.015 to 0.025, in one embodiment, 0.016 to 0.025, in one embodiment, 0.017 to 0.025, in one embodiment, 0.018 to 0.025, in one embodiment, 0.019 to 0.025, in one embodiment, 0.02 to 0.025, in one embodiment, 0.016 to 0.02, in one embodiment, 0.005 to 0.04, and in one embodiment 0.01 to 0.03.

In the chromatogram obtained by high performance liquid chromatography under the above conditions, the area ratio (decaprenylphenol/RQ9) of the peak containing decaprenylphenol to the peak containing RQ9 is not particularly limited, but, it may be, for example, 0.1 or more, in one embodiment, 0.2 or more, in one embodiment, 0.3 or more, in one embodiment, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.2 or more, in one embodiment, 1.4 or more, in one embodiment, 1.6 or more, in one embodiment, 1.8 or more, in one embodiment, 2.0 or more, in one embodiment, 2.2 or more, in one embodiment, 2.3 or more, in one embodiment, 2.4 or more, in one embodiment, 2.6 or more, in one embodiment, 2.8 or more, in one embodiment, 3.0 or more, in one embodiment, 3.2 or more, in one embodiment, 3.4 or more, or in one embodiment, 3.6 or more, and the upper limit may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.8 or less, or in one embodiment, 3.7 or less.

In the chromatogram obtained by high performance liquid chromatography under the above conditions, the area ratio (decaprenylphenol/RQ9) of the peak containing decaprenylphenol to the peak containing RQ9 is not particularly limited, but may be 0.1 to 10.0, 0.1 to 8.0, 0.1 to 6.0, 0.1 to 5.0, 0.1 to 4.0, 0.5 to 4.0, or 1.0 to 4.0.

In the chromatogram obtained by high performance liquid chromatography under the above conditions, the area ratio (decaprenylphenol/RQ9) of the peak containing decaprenylphenol to the peak containing RQ9 is not particularly limited, but may be, for example, within the range of 0.1 to 10.0, in one embodiment, 0.1 to 9.0, in one embodiment, 0.1 to 8.0, in one embodiment, 0.1 to 7.0, in one embodiment, 0.1 to 6.0, in one embodiment, 0.1 to 5.0, in one embodiment, 0.1 to 4.0, in one embodiment, 0.2 to 4.0, in one embodiment, 0.3 to 4.0, in one embodiment, 0.4 to 4.0, in one embodiment, 0.5 to 4.0, in one embodiment, 0.6 to 4.0, in one embodiment, 0.7 to 4.0, in one embodiment, 0.8 to 4.0, in one embodiment, 0.9 to 4.0, in one embodiment, 1.0 to 4.0, in one embodiment, 1.2 to 4.0, in one embodiment, 1.4 to 4.0, in one embodiment, 1.6 to 4.0, in one embodiment, 1.8 to 4.0, in one embodiment, 2.0 to 4.0, in one embodiment, 2.2 to 4.0, in one embodiment, 2.3 to 4.0, in one embodiment, 2.4 to 4.0, in one embodiment, 2.6 to 4.0, in one embodiment, 2.8 to 4.0, in one embodiment, 3.0 to 4.0, in one embodiment, 3.2 to 4.0, in one embodiment, 3.4 to 4.0, in one embodiment, 3.6 to 4.0, in one embodiment, 3.6 to 3.8, or in one embodiment, 3.6 to 3.7.

In a chromatogram obtained by high performance liquid chromatography under the above conditions, the area ratio (RQ11/RQ9) of a peak containing RQ11 to a peak containing RQ9 is not particularly limited, but may be, for example, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, in one embodiment, 2.0 or more, in one embodiment, 2.2 or more, in one embodiment, 2.4 or more, in one embodiment, 2.6 or more, in one embodiment, 2.8 or more, in one embodiment, 3.0 or more, in one embodiment, 3.2 or more, in one embodiment, 3.3 or more, in one embodiment, 3.4 or more, in one embodiment, 3.6 or more, in one embodiment, 3.8 or more, in one embodiment, 4.0 or more, in one embodiment, 4.2 or more, in one embodiment, 4.4 or more, in one embodiment, 4.6 or more, in one embodiment, 4.8 or more, in one embodiment, 5.0 or more, in one embodiment, 5.5 or more, in one embodiment, 6.0 or more, in one embodiment, 6.5 or more, in one embodiment, 7.0 or more, in one embodiment, 7.5 or more, or in one embodiment 8.0 or more, and may be, for example, 15.0 or less, in one embodiment, 12.0 or less, in one embodiment, 10.0 or less, in one embodiment, 9.5 or less, in one embodiment, 9.0 or less, in one embodiment, 8.5 or less, in one embodiment, 8.4 or less, in one embodiment, 8.3 or less, in one embodiment, 8.2 or less, or in one embodiment, 8.1 or less.

In the chromatogram obtained by high performance liquid chromatography under the above conditions, the area ratio (RQ11/RQ9) of the peak containing RQ11 to the peak containing RQ9 is not particularly limited, but may be within the range of 1.0 to 15.0, 1.2 to 15.0, 1.4 to 12.0, 1.5 to 10.0, 1.8 to 9.5, or 2.0 to 9.0.

The area ratio (RQ11/RQ9) of the peak containing RQ11 to the peak containing RQ9 in the chromatogram obtained by high performance liquid chromatography under the above conditions is not particularly limited, but, for example, may be within the range of 1.0 to 15.0, in one embodiment, 1.0 to 12.0, in one embodiment, 1.0 to 10.0, in one embodiment, 1.0 to 9.5, in one embodiment, 1.0 to 9.0, in one embodiment, 1.0 to 8.5, in one embodiment, 1.0 to 8.4, in one embodiment, 1.0 to 8.3, in one embodiment, 1.0 to 8.2, in one embodiment, 1.0 to 8.1, in one embodiment, 1.1 to 8.1, in one embodiment, 1.2 to 8.1, in one embodiment, 1.3 to 8.1, in one embodiment, 1.4 to 8.1, in one embodiment, 1.5 to 8.1, in one embodiment, 1.6 to 8.1, in one embodiment, 1.7 to 8.1, in one embodiment, 1.8 to 8.1, in one embodiment, 1.9 to 8.1, in one embodiment, 2.0 to 8.1, in one embodiment, 2.2 to 8.1, in one embodiment, 2.4 to 8.1, in one embodiment, 2.6 to 8.1, in one embodiment, 2.8 to 8.1, in one embodiment, 3.0 to 8.1, in one embodiment, 3.2 to 8.1, in one embodiment, 3.3 to 8.1, in one embodiment, 3.4 to 8.1, in one embodiment, 3.6 to 8.1, in one embodiment, 3.8 to 8.1, in one embodiment, 4.0 to 8.1, in one embodiment, 4.2 to 8.1, in one embodiment, 4.4 to 8.1, in one embodiment, 4.6 to 8.1, in one embodiment, 4.8 to 8.1, in one embodiment, 5.0 to 8.1, in one embodiment, 5.5 to 8.1, in one embodiment, 6.0 to 8.1, in one embodiment, 6.5 to 8.1, in one embodiment, 7.0 to 8.1, in one embodiment, 7.5 to 8.1, in one embodiment, 8.0 to 8.1, in one embodiment, 1.2 to 15.0, in one embodiment, 1.4 to 12.0, in one embodiment, 1.5 to 10.0, in one embodiment, 1.8 to 9.5, or in one embodiment 2.0 to 9.0.

In the chromatogram obtained by high performance liquid chromatography under the above conditions, the area ratio (decaprenylphenol/RD) of the peak containing decaprenylphenol to the peak containing RD is not particularly limited and may be, for example, 0.1 or more, in one embodiment, 0.5 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, and may be, in one embodiment, 10 or less, in one embodiment, 5.0 or less, in one embodiment, 3.0 or less, in one embodiment, 2.5 or less, and in one embodiment, 2.0 or less.

In the chromatogram obtained by high performance liquid chromatography under the above conditions, the area ratio (decaprenylphenol/RD) of the peak containing decaprenylphenol to the peak containing RD is not particularly limited, but is within the range of preferably 0.1 to 10, more preferably 0.5 to 5.0, and still more preferably 1.0 to 3.0.

In the chromatogram obtained by high performance liquid chromatography under the above conditions, the area ratio (decaprenylphenol/RD) of the peak containing decaprenylphenol to the peak containing RD is not particularly limited, but, for example, is within the range of 0.1 to 10, in one embodiment, 0.5 to 10, in one embodiment, 0.7 to 10, in one embodiment, 0.7 to 5.0, in one embodiment, 0.7 to 3.0, in one embodiment, 0.7 to 2.5, in one embodiment, 0.7 to 2.0, in one embodiment, 0.8 to 2.0, in one embodiment, 0.9 to 2.0, in one embodiment, 1.0 to 2.0, in one embodiment, 1.1 to 2.0, in one embodiment, 1.2 to 2.0, in one embodiment, 1.3 to 2.0, in one embodiment, 1.4 to 2.0, in one embodiment, 1.5 to 2.0, in one embodiment, 1.6 to 2.0, in one embodiment, 1.7 to 2.0, in one embodiment, 1.8 to 2.0, in one embodiment, 0.5 to 5.0, and in one embodiment, 1.0 to 3.0.

In the chromatogram obtained by high performance liquid chromatography under the above conditions, the area ratio (RD/RQ11) of the peak containing RD to the peak containing RQ11 is not particularly limited, but, for example, it is 0.1 or more, in one embodiment, 0.2 or more, specifically, 0.19 or more, in one embodiment, 0.20 or more, in one embodiment, 0.21 or more, and in one embodiment, 1.0 or less, in one embodiment, 0.8 or less, in one embodiment, 0.5 or less, specifically, 0.50 or less, in one embodiment, 0.49 or less, in one embodiment, 0.48 or less, and in one embodiment, 0.47 or less.

In the chromatogram obtained by high performance liquid chromatography under the above conditions, the area ratio (RD/RQ11) of the peak containing RD to the peak containing RQ11 is not particularly limited, but is within the range of preferably 0.1 to 1.0, more preferably 0.2 to 0.8, and still more preferably 0.2 to 0.5.

In the chromatogram obtained by high performance liquid chromatography under the above conditions, the area ratio (RD/RQ11) of the peak containing RD to the peak containing RQ11 is not particularly limited, but is, for example, within the range of 0.1 to 1.0, in one embodiment, 0.2 to 0.8, in one embodiment, 0.2 to 0.5, specifically, 0.19 to 0.50, in one embodiment, 0.20 to 0.49, in one embodiment, 0.21 to 0.48, and in one embodiment, 0.21 to 0.47.

In the chromatogram obtained by high performance liquid chromatography under the above conditions, the area ratio (decaprenylphenol/RQ11) of the peak containing decaprenylphenol to the peak containing RQ11 is not particularly limited, but may be, for example, 0.01 or more, in one embodiment, 0.03 or more, in one embodiment, 0.05 or more, in one embodiment, 0.07 or more, in one embodiment, 0.08 or more, in one embodiment, 0.1 or more, in one embodiment, 0.2 or more, in one embodiment, 0.25 or more, in one embodiment, 0.3 or more, in one embodiment, 0.35 or more, in one embodiment, 0.4 or more, or in one embodiment, 0.45 or more, and may be, for example, 1.5 or less, in one embodiment, 1.1 or less, in one embodiment, 1.0 or less, in one embodiment 0.8 or less, in one embodiment, 0.7 or less, in one embodiment, 0.6 or less, or in one embodiment 0.5 or less.

In the chromatogram obtained by high performance liquid chromatography under the above conditions, the area ratio (decaprenylphenol/RQ11) of the peak containing decaprenylphenol to the peak containing RQ11 is not particularly limited, but may be within the range of 0.01 to 1.5, 0.03 to 1.0, 0.05 to 0.8, 0.07 to 0.7, 0.08 to 0.6, or 0.1 to 0.5.

In the chromatogram obtained by high performance liquid chromatography under the above conditions, the area ratio (decaprenylphenol/RQ11) of the peak containing decaprenylphenol to the peak containing RQ11 is not particularly limited, but may be, for example, within the range of 0.01 to 1.5, in one embodiment, 0.03 to 1.5, in one embodiment, 0.05 to 1.5, in one embodiment, 0.07 to 1.5, in one embodiment, 0.08 to 1.5, in one embodiment, 0.1 to 1.5, in one embodiment, 0.2 to 1.5, in one embodiment, 0.2 to 1.1, in one embodiment, 0.2 to 1.0, in one embodiment, 0.2 to 0.8, in one embodiment, 0.2 to 0.7, in one embodiment, 0.2 to 0.6, in one embodiment, 0.2 to 0.5, in one embodiment, 0.25 to 0.5, in one embodiment, 0.3 to 0.5, in one embodiment, 0.35 to 0.5, in one embodiment, 0.4 to 0.5, in one embodiment, 0.45 to 0.5, in one embodiment, 0.03 to 1.0, in one embodiment, 0.05 to 0.8, in one embodiment, 0.07 to 0.7, in one embodiment, 0.08 to 0.6, or in one embodiment 0.1 to 0.5.

In the chromatogram obtained by high performance liquid chromatography under the above conditions, the ratio ((decaprenylphenol+RQ9)/QH) of the total area of the peak containing decaprenylphenol and the peak containing reduced coenzyme Q9 (RQ9) to the area of the peak containing reduced coenzyme Q10 (QH) is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.025 or more, in one embodiment, 0.030 or more, in one embodiment, 0.10 or less, in one embodiment, 0.050 or less, in one embodiment, 0.040 or less, in one embodiment, 0.030 or less, in one embodiment, 0.020 or less, in one embodiment, 0.025 or less, in one embodiment, 0.020 or less, and in one embodiment, 0.015 or less.

In the chromatogram obtained by high performance liquid chromatography under the above conditions, the ratio ((decaprenylphenol+RQ9)/QH) of the total area of the peak containing decaprenylphenol and the peak containing reduced coenzyme Q9 (RQ9) to the area of the peak containing reduced coenzyme Q10 (QH) is not particularly limited, but is, for example, within the range of 0.001 to 0.10, in one embodiment, 0.005 to 0.050, in one embodiment, 0.007 to 0.040, in one embodiment, 0.008 to 0.030, in one embodiment, 0.009 to 0.025, in one embodiment, 0.010 to 0.020, and in one embodiment 0.010 to 0.015.

In the chromatogram obtained by high performance liquid chromatography under the above conditions, the ratio ((decaprenylphenol+RQ11)/QH) of the total area of the peak containing decaprenylphenol and the peak containing reduced coenzyme Q11 (RQ11) to the area of the peak containing reduced coenzyme Q10 (QH) is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.025 or more, in one embodiment, 0.030 or more, in one embodiment, 0.10 or less, in one embodiment, 0.050 or less, in one embodiment, 0.040 or less, in one embodiment, 0.035 or less, in one embodiment, 0.032 or less, and in one embodiment, 0.031 or less.

In the chromatogram obtained by high performance liquid chromatography under the above conditions, the ratio ((decaprenylphenol+RQ11)/QH) of the total area of the peak containing decaprenylphenol and the peak containing reduced coenzyme Q11 (RQ11) to the peak containing reduced coenzyme Q10 (QH) is not particularly limited but is, for example, within the range of 0.001 to 0.10, in one embodiment, 0.005 to 0.050, in one embodiment, 0.007 to 0.040, in one embodiment, 0.008 to 0.035, in one embodiment, 0.009 to 0.032, in one embodiment, 0.010 to 0.031, in one embodiment, 0.012 to 0.031, in one embodiment, 0.013 to 0.031, in one embodiment, 0.014 to 0.031, in one embodiment, 0.015 to 0.031, in one embodiment, 0.020 to 0.031, and in one embodiment, 0.025 to 0.031.

In the chromatogram obtained by high performance liquid chromatography under the above conditions, the ratio ((RQ9+RQ11)/QH) of the total area of the peaks containing reduced coenzyme Q9 (RQ9) and the peaks containing reduced coenzyme Q11 (RQ11) to the area of the peak containing reduced coenzyme Q10 (QH) is not particularly limited, but, for example, may be 0.001 or more, in one embodiment, 0.005 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.021 or more, in one embodiment, 0.022 or more, and may be, in one embodiment, 0.10 or less, in one embodiment, 0.050 or less, in one embodiment, 0.040 or less, in one embodiment, 0.035 or less, in one embodiment, 0.030 or less, in one embodiment, 0.029 or less, in one embodiment, 0.028 or less, in one embodiment, 0.027 or less, in one embodiment, 0.026 or less, and in one embodiment 0.025 or less.

In the chromatogram obtained by high performance liquid chromatography under the above conditions, the ratio ((RQ9+RQ11)/QH) of the total area of the peak containing reduced coenzyme Q9 (RQ9) and the peak containing reduced coenzyme Q11 (RQ11) to the peak containing reduced coenzyme Q10 (QH) is not particularly limited, but is, for example, within the range of 0.001 to 0.10, in one embodiment, 0.005 to 0.050, in one embodiment, 0.010 to 0.040, in one embodiment, 0.011 to 0.035, in one embodiment, 0.012 to 0.030, in one embodiment, 0.013 to 0.029, in one embodiment, 0.014 to 0.028, in one embodiment, 0.014 to 0.027, in one embodiment, 0.015 to 0.026, in one embodiment, 0.016 to 0.026, in one embodiment, 0.017 to 0.026, in one embodiment, 0.018 to 0.026, in one embodiment, 0.020 to 0.026, and in one embodiment, 0.022 to 0.026.

In the present embodiment, in a chromatogram obtained by high performance liquid chromatography under the above conditions, the area ratio (total value of the peak areas of the QH analogs/peak area of QH) of peaks containing QH analogs (RD, RQ11, RQ9, and decaprenylphenol) to a peak containing QH is not particularly limited, but, for example, may be 0.001 or more, in one embodiment, 0.006 or more, in one embodiment, 0.007 or more, in one embodiment, 0.012 or more, in one embodiment, 0.02 or more, in one embodiment, 0.022 or more, in one embodiment, 0.024 or more, in one embodiment, 0.03 or more, in one embodiment, 0.033 or more, in one embodiment, 0.035 or more, in one embodiment, 0.038 or more, in one embodiment, 0.10 or less, in one embodiment, 0.08 or less, in one embodiment, 0.06 or less, in one embodiment, 0.05 or less, and in one embodiment 0.04 or less.

In the present embodiment, in the chromatogram obtained by high performance liquid chromatography under the above conditions, the area ratio (total value of the peak areas of QH analogs/peak area of QH) of the peaks containing QH analogs (RD, RQ11, RQ9, and decaprenylphenol) to the peak containing QH is not particularly limited, but is within the range of preferably 0.001 to 0.10, more preferably 0.006 to 0.08, still more preferably 0.007 to 0.06, particularly preferably 0.012 to 0.05, and most preferably 0.02 to 0.04.

In the present embodiment, in the chromatogram obtained by high performance liquid chromatography under the above conditions, the area ratio (total value of the peak areas of QH analogs/peak area of QH) of the peaks containing QH analogs (RD, RQ11, RQ9, and decaprenylphenol) to the peak containing QH is not particularly limited, but is, for example, within the range of 0.001 to 0.10, in one embodiment, 0.001 to 0.08, in one embodiment, 0.001 to 0.06, in one embodiment, 0.001 to 0.05, in one embodiment, 0.001 to 0.04, in one embodiment, 0.006 to 0.04, in one embodiment, 0.007 to 0.04, in one embodiment, 0.012 to 0.04, in one embodiment, 0.02 to 0.04, in one embodiment, 0.022 to 0.04, in one embodiment, 0.024 to 0.04, in one embodiment, 0.03 to 0.04, in one embodiment, 0.033 to 0.04, in one embodiment, 0.035 to 0.04, in one embodiment, 0.038 to 0.04, in one embodiment, 0.006 to 0.08, in one embodiment, 0.007 to 0.06, and in one embodiment 0.012 to 0.05.

The compound may further contain substances other than reduced coenzyme Q10 or its analogs, reduced demethoxy Q10, or reduced coenzyme Q9. Examples of such substances include an excipient, a disintegrant, a lubricant, a binder, an emulsifier, a colorant, an anti-caking agent, an absorption enhancer, a dissolution aid, a stabilizer, a flavoring agent, fats and oils, a surfactant, a higher fatty acid, ethanol, water, an active ingredient other than reduced coenzyme Q10, an antioxidant.

The form of the composition is not particularly limited, and can be appropriately selected as necessary. Specifically, examples thereof include oral preparations such as tablets, powders, chewable tablets, pills, capsules, granules, fine granules, sustained-release preparations, suspensions, emulsion preparations, syrups, elixirs, and the like, and non-oral preparations such as injections, suppositories, topical preparations, patch preparations, and the like. Since reduced coenzyme Q10 has excellent oral absorbability, the form of an oral preparation is particularly preferred. Among these, the composition in solid form is preferred. Hereinafter, the case where reduced coenzyme Q10 crystal is caused to coexist with reduced demethoxy Q10 and reduced coenzyme Q9 in the composition in solid form such as granules or tablets will be described.

The form of the composition in solid form is not particularly limited, and examples thereof include forms such as powder, granules, tablet, hard capsule, and the like.

The type of emulsifier to be added to the composition in solid form is not particularly limited, but the use of an emulsifier that is acceptable for food, cosmetic, and/or pharmaceutical use is particularly preferred. For example, the use of an emulsifier having an HLB of 1 or more and 17 or less, preferably 2 or more and 16 or less, is possible. The use of two or more emulsifiers in combination is allowed. The preferably used specific examples of emulsifiers include one or two or more emulsifiers selected from the group consisting of glycerin fatty acid ester, sucrose fatty acid ester, retinol fatty acid ester, propylene glycol fatty acid ester, sorbitan fatty acid ester, and polysorbate.

From the viewpoint of suppressing the oxidation of reduced coenzyme Q10, among the above emulsifiers, emulsifiers that are neither in powder nor flake form are preferably used. Examples of the emulsifiers that are neither in powder nor flake form include emulsifiers that are in a liquid-to-sol form, a gel form, or a soft solid, and emulsifiers that are liquid, viscid liquid, viscous liquid, paste, pellets, waxy mass, wax, soft solid, or semi-solid are more preferred. As the emulsifier, an emulsifier having the above physical properties at 50° C. is preferred, and an emulsifier having the above physical properties at 25° C. is most preferred.

Examples of the emulsifiers that are neither in powder nor in flake form include emulsifiers in which the melting onset point in a measurement by a differential scanning calorimeter (DSC) with the heating rate set to 1° C./min or more and 20° C./min or less is 50° C. or lower, preferably 40° C. or lower, more preferably 25° C. or lower.

From another viewpoint, examples of the emulsifiers include emulsifiers in which the viscosity, in a measurement with the use of a B-type viscometer at a rotational speed of 10 rpm and a sample temperature of 50° C., is, for example, 150,000 mPa·s or less, preferably 100,000 mPa·s or less, more preferably 50,000 mPa·s or less, more preferably 30,000 mPa·s or less, and most preferably 25,000 mPa·s or less. The lower limit of the viscosity is not particularly limited as long as the viscosity is greater than 0 mPa·s, the viscosity is more preferably 1 mPa·s or more, particularly preferably 5 mPa·s or more, and most preferably 10 mPa·s or more. That is, the viscosity can be, for example, within the range of 1 to 150,000 mPa·s, preferably within the range of 5 to 30,000 mPa·s, and particularly preferably within the range of 10 to 25,000 mPa·s.

The specific examples of the emulsifier include one or more selected from an ester compound of a polyol selected from monoglycerin, polyglycerin, sorbitan, polyoxyethylene sorbitan, sucrose, propylene glycol, polypropylene glycol, ethylene glycol, and polyethylene glycol, and a fatty acid which may have a substituent, and lecithin.

In polyglycerin, the number of units of glycerin may be 2 or more, and is preferably 2 or more and 10 or less. For example, diglycerin, triglycerin, tetraglycerin, pentaglycerin, hexaglycerin, and decaglycerin can be exemplified.

In polyoxyethylene sorbitan, the number of units of oxyethylene may be 2 or more, preferably 10 or more and 30 or less, more preferably 15 or more and 25 or less.

In polypropylene glycol, the number of units of propylene glycol may be 2 or more, and is preferably 2 or more and 10 or less.

In polyethylene glycol, the number of units of ethylene glycol may be 2 or more, and preferably is 2 or more and 10 or less.

Examples of the fatty acids that may have a substituent include straight-chain or branched-chain monocarboxylic or dicarboxylic fatty acids having 4 or more and 24 or less carbon atoms. As substituents, a hydroxy group and an acetoxy group can be exemplified. It is preferred that the number of substituents is 2 or less. Examples of the fatty acids that may have a substituent include lauric acid, oleic acid, caprylic acid, stearic acid, behenic acid, ricinoleic acid, succinic acid, and diacetyl tartaric acid.

In the ester compound of the polyol and the fatty acid, the number of bonds of the fatty acid to one molecule of the polyol is not particularly limited, and can be appropriately adjusted according to the HLB of the emulsifier for the intended purpose.

From the viewpoint of suppressing oxidation of reduced coenzyme Q10, the number of bonds of the fatty acid to one molecule of the polyol can be, for example, 12 or less, preferably 10 or less, more preferably 7 or less, more preferably 6 or less, more preferably 5 or less, more preferably 3 or less, more preferably 2 or less, and more preferably, is 1.

Specific examples of the ester compound of the polyol and the fatty acid include diglyceryl monooleate, monoglyceryl monocaprylate, diglyceryl monocaprylate, decaglyceryl pentaoleate, tetraglyceryl pentaoleate, pentaglyceryl trioleate, decaglyceryl monolaurate, hexaglyceryl monocaprylate, hexaglyceryl monooleate, pentaglyceryl monostearate, tetraglyceryl tristearate, decaglyceryl monobehenate, monostearate of mono- and diglycerides, monoglyceryl monooleate, glyceryl monostearate succinate, monoglyceryl succinate, glyceryl monostearate diacetyl tartrate, propylene glycol monooleate, sorbitan monooleate, sorbitan monostearate, sorbitan tristearate, monoglyceryl monolaurate, diglyceryl monolaurate, diglyceryl monomyristate, tetraglyceryl pentastearate, polyoxyethylene sorbitan monooleate, polyoxyethylene sorbitan monolaurate, polyoxyethylene sorbitan monostearate, condensed ricinoleic acid pentaglyceride, sucrose stearate, sucrose erucate, and sucrose oleate.

From the viewpoint of suppressing oxidation of reduced coenzyme Q10, the fatty acid in the ester compound of the polyol and the fatty acid is more preferably an unsaturated fatty acid. Examples of the unsaturated fatty acid include, for example, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, alpha-linolenic acid, gamma-linolenic acid, pinolenic acid, alpha-eleostearic acid, beta-eleostearic acid, Mead acid, dihomo-gamma-linolenic acid, eicosatrienoic acid, stearidonic acid, arachidonic acid, eicosatetraenoic acid, adrenic acid, boseopentaenoic acid, eicosapentaenoic acid, Osbond acid, clupanodonic acid, tetracosapentaenoic acid, docosahexaenoic acid, cetoleic acid, ricinoleic acid, and condensed ricinoleic acid, and oleic acid, condensed ricinoleic acid, linoleic acid, and erucic acid are more preferred, and oleic acid is particularly preferred.

Specific examples of the ester compounds of the polyol and the unsaturated fatty acid include monoglyceryl monooleate, monoglyceryl mono- and dioleate, monoglyceryl dioleate, mono- and diglyceryl monooleate, diglyceryl monooleate, diglyceryl mono- and dioleate, diglyceryl dioleate, diglyceryl trioleate, triglyceryl monooleate, triglyceryl dioleate, triglyceryl trioleate, triglyceryl tetraoleate, tetraglyceryl monooleate, tetraglyceryl dioleate, tetraglyceryl trioleate, tetraglyceryl tetraoleate, tetraglyceryl pentaoleate, pentaglyceryl monooleate, pentaglyceryl dioleate, pentaglyceryl trioleate, pentaglyceryl tetraoleate, pentaglyceryl pentaoleate, pentaglyceryl hexaoleate, hexaglyceryl monooleate, hexaglyceryl dioleate, hexaglyceryl trioleate, hexaglyceryl tetraoleate, hexaglyceryl pentaoleate, hexaglyceryl hexaoleate, hexaglyceryl heptaoleate, decaglyceryl monooleate, decaglyceryl dioleate, decaglyceryl trioleate, decaglyceryl tetraoleate, decaglyceryl pentaoleate, decaglyceryl hexaoleate, decaglyceryl heptaoleate, decaglyceryl octaoleate, decaglyceryl nonaoleate, decaglyceryl decaoleate, decaglyceryl dodecaoleate, diacetylglycerin monooleate, glyceryl monooleate lactate, glyceryl monooleate succinate, glyceryl monooleate citrate, glyceryl monooleate diacetyl tartrate, sucrose oleate, propylene glycol monooleate, sorbitan monooleate, sorbitan dioleate, sorbitan trioleate, polyoxyethylene sorbitan monooleate, phosphatidylcholine monopalmitate monooleate, phosphatidylcholine dilinoleate, monoglyceryl monoerucate, monoglyceryl mono- and dierucate, monoglyceryl dierucate, mono- and diglyceryl monoerucate, diglyceryl monoerucate, diglyceryl mono- and dierucate, diglyceryl dierucate, diglyceryl trierucate, triglyceryl monoerucate, triglyceryl dierucate, triglyceryl trierucate, triglyceryl tetraerucate, tetraglyceryl monoerucate, tetraglyceryl dierucate, tetraglyceryl trierucate, tetraglyceryl tetraerucate, tetraglyceryl pentaerucate, pentaglyceryl monoerucate, pentaglyceryl dierucate, pentaglyceryl trierucate, pentaglyceryl tetraerucate, pentaglyceryl pentaerucate, pentaglyceryl hexaerucate, hexaglyceryl monoerucate, hexaglyceryl dierucate, hexaglyceryl trierucate, hexaglyceryl tetraerucate, hexaglyceryl pentaerucate, hexaglyceryl hexaerucate, hexaglyceryl heptaerucate, decaglyceryl monoerucate, decaglyceryl dierucate, decaglyceryl trierucate, decaglyceryl tetraerucate, decaglyceryl pentaerucate, decaglyceryl hexaerucate, decaglyceryl heptaerucate, decaglyceryl octaerucate, decaglyceryl nonaerucate, decaglyceryl decaerucate, decaglyceryl dodecaerucate, diacetylglycerin monoerucate, glyceryl monoerucate lactate, glyceryl monoerucate succinate, glyceryl monoerucate citrate, glyceryl monoerucate diacetyl tartrate, sucrose erucate, propylene glycol monoerucate, sorbitan monoerucate, sorbitan dierucate, sorbitan trierucate, condensed ricinoleic acid monoglyceride, condensed ricinoleic acid diglyceride, condensed ricinoleic acid triglyceride, condensed ricinoleic acid tetraglyceride, condensed ricinoleic acid pentaglyceride, condensed ricinoleic acid hexaglyceride, condensed ricinoleic acid heptaglyceride, and condensed ricinoleic acid decaglyceride.

From the viewpoint of suppression of oxidation of reduced coenzyme Q10, as the ester compound of the polyol and the fatty acid, a polyoxyethylene sorbitan fatty acid ester is also preferably used. Specific examples of the polyoxyethylene sorbitan fatty acid ester include polyoxyethylene sorbitan monooleate, polyoxyethylene sorbitan monolaurate, and polyoxyethylene sorbitan monostearate.

The composition preferably contains an antioxidant in order to ensure oxidative stability, particularly in a solid dosage form. Here, the type of the antioxidant is not particularly limited. The use of two or more kinds of antioxidants in combination is permitted. Specific examples of antioxidants preferably include those that are solid at room temperature, and examples thereof include one or more antioxidants selected from the group consisting of ascorbic acid, ascorbate, erythorbic acid, and erythorbate.

The counterion of each of ascorbate and erythorbate is not limited, but can be one or more metal salts, each independently selected from the group consisting of sodium salts, potassium salts, calcium salts, and magnesium salts.

As an antioxidant, the use of an antioxidant acceptable in foods or pharmaceuticals is particularly preferred. As the antioxidant, an ascorbate is preferred, and it is particularly preferred that the ascorbate be one or more selected from sodium ascorbate and calcium ascorbate.

The composition preferably further contains a binder. The binder can be utilized to bind respective components such as reduced coenzyme Q10, an emulsifier, an antioxidant, and the like, and to form a composition in a solid form.

The type of binder is not limited. Use may be made of two or more binders in combination. Specific examples of binders include one or more selected from celluloses and starches.

Examples of the celluloses include hydroxypropyl cellulose, hydroxypropyl methylcellulose, hydroxymethyl cellulose, carboxymethyl cellulose, crystalline cellulose, cellulose powder, methyl cellulose, ethyl cellulose, and salts thereof and the like. A particularly preferred binder is one or more selected from the group consisting of hydroxypropyl cellulose, hydroxypropyl methylcellulose, and sodium carboxymethyl cellulose.

Examples of the starches include wheat starch, potato starch, sweet potato starch, corn starch, dextrin, hydroxypropyl starch, acetate starch, oxidized starch, partially alpha-converted starch, and the like.

As a binder, the use of a binder that is acceptable as a food or a pharmaceutical is particularly preferred.

The blending proportion of each component in the composition can be appropriately set according to the dosage form and the like.

In a case where the composition contains an antioxidant, the content of the antioxidant is, for example, 1 part by weight or more and 9900 parts by weight or less, with respect to 100 parts by weight of reduced coenzyme Q10. The content of the antioxidant in the composition is, with respect to 100 parts by weight of reduced coenzyme Q10, preferably 20 parts by weight or more, more preferably 50 parts by weight or more, more preferably 55 parts by weight or more, and preferably 5000 parts by weight or less, more preferably 1000 parts by weight or less, more preferably 500 parts by weight or less, more preferably 200 parts by weight or less, and more preferably 120 parts by weight or less. The content of the antioxidant is, with respect to 100 parts by weight of reduced coenzyme Q10, with the range of preferably 20 to 5000 parts by weight, more preferably 50 to 1000 parts by weight, more preferably 55 to 500 parts by weight, more preferably 55 to 200 parts by weight and particularly preferably 55 to 120 parts by weight.

In the case where the composition contains an emulsifier, the content of the emulsifier is, for example, from 1 part by weight or more to 9900 parts by weight or less, with respect to 100 parts by weight of reduced coenzyme Q10. The content of the emulsifier in the composition is, with respect to 100 parts by weight of reduced coenzyme Q10, preferably 3 parts by weight or more, more preferably 5 parts by weight or more, and more preferably 8 parts by weight or more, and preferably 5000 parts by weight or less, more preferably 1000 parts by weight or less, more preferably 500 parts by weight or less, more preferably 200 parts by weight or less, more preferably 150 parts by weight or less, more preferably 100 parts by weight or less, more preferably 50 parts by weight or less, more preferably 30 parts by weight or less. The content of an emulsifier is, with respect to 100 parts by weight of reduced coenzyme Q10, within the range of preferably 3 to 5000 parts by weight, more preferably 5 to 1000 parts by weight, more preferably 8 to 500 parts by weight, more preferably 8 to 200 parts by weight, more preferably 8 to 150 parts by weight, more preferably 8 to 100 parts by weight, more preferably 8 to 50 parts by weight, and particularly preferably 8 to 30 parts by weight.

In the case where the composition contains a binder, the composition contains, with respect to 100 parts by weight of reduced coenzyme Q10, a binder in a proportion of, for example, 1 part by weight or more, preferably 3 parts by weight or more, more preferably 5 parts by weight or more, particularly preferably 8 parts by weight or more, and still more preferably 10 parts by weight or more. Also, the composition contains, with respect to 100 parts by weight of reduced coenzyme Q10, a binder in a proportion of, for example, 600 parts by weight or less, preferably 550 parts by weight or less, more preferably 500 parts by weight or less, more preferably 450 parts by weight or less, and still more preferably 400 parts by weight or less. The content of a binder is, with respect to 100 parts by weight of reduced coenzyme Q10, for example, within the range of 1 to 600 parts by weight, preferably 3 to 550 parts by weight, more preferably 5 to 500 parts by weight, particularly preferably 8 to 450 parts by weight, and still preferably 10 to 400 parts by weight.

The content of the antioxidant in the composition is, with respect to the total weight of the composition, for example, 1 wt % or more, preferably 2 wt % or more, more preferably 4 wt % or more, more preferably 5 wt % or more, and for example, 99 wt % or less, preferably 90 wt % or less, preferably 80 wt % or less, more preferably 70 wt % or less, more preferably 60 wt % or less. The content of the antioxidant can be, with respect to the total weight of the composition, within the range of, for example, 1 to 99 wt %, preferably 2 to 90 wt %, more preferably 4 to 80 wt %, more preferably 5 to 70 wt %, and particularly preferably 5 to 60 wt %.

The content of the emulsifier in the composition is, with respect to the total weight of the composition, for example, 0.5 wt % or more, preferably 0.7 wt % or more, more preferably 0.9 wt % or more, and for example, 99 wt % or less, preferably 50 wt % or less, more preferably 30 wt % or less, more preferably 25 wt % or less, more preferably 20 wt % or less, more preferably 15 wt % or less, more preferably 10 wt % or less. The content of the emulsifier can be, with respect to the total weight of the composition, within the range of, for example, 0.5 to 99 wt %, preferably 0.7 to 50 wt %, more preferably 0.9 to 30 wt %, more preferably 0.9 to 25 wt %, more preferably 0.9 to 20 wt %, more preferably 0.9 to 15 wt %, and more preferably 0.9 to 10 wt %.

The content of the binder in the composition is, with respect to the total weight of the composition, for example, 1 wt % or more, preferably 10 wt % or more, more preferably 20 wt % or more, and for example, 80 wt % or less, preferably 70 wt % or less, more preferably 60 wt % or less. The content of the binder can be, with respect to the total weight of the composition, within the range of, for example, 1 to 80 wt %, preferably 10 to 70 wt %, more preferably 20 to 60 wt %.

The step of causing coexistence, in a composition, of the reduced coenzyme Q10 crystal with reduced demethoxy Q10 and reduced coenzyme Q9 can comprise separating a composition in solid form that contains reduced coenzyme Q10, reduced demethoxy Q10, and reduced coenzyme Q9 from a solution containing the reduced coenzyme Q10, reduced demethoxy Q10, and reduced coenzyme Q9. By controlling the blending ratio of each component in the solution and the conditions of the separation step so that the peak area ratio in the chromatogram for RD and RQ9 in the composition to be separated is within the specific range, the polymorphic stability of the reduced coenzyme Q10 (QH) crystal can improve. Specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   the content of each component in the solution and the conditions of the separation step are preferably controlled so that an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may become 0.3 or more. The content of each component in the solution and the conditions of the separation step are preferably controlled so that the above peak area ratio (RD/RQ9) may become 0.5 or more, 0.8 or more, 1.0 or more, 1.5 or more, or 2.0 or more. Furthermore, the content of each component in the solution and the conditions of the separation step may be controlled so that the above peak area ratio (RD/RQ9) may become 10.0 or less, 8.0 or less, 6.0 or less, 4.0 or less, or 3.0 or less. The content of each component in the solution and the conditions of the separation step may be controlled so that the above peak area ratio (RD/RQ9) may become within the range of 0.3 to 10.0, 0.5 to 10.0, 0.5 to 3.0, 0.8 to 8.0, 1.0 to 6.0, 1.0 to 2.0, 1.5 to 4.0, or 2.0 to 3.0.

In another embodiment, specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   the content of each component in the solution and the conditions of the separation step are controlled so that an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may become 0.3 or more. The above peak area ratio (RD/RQ9) may be, for example, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, or in one embodiment, 2.0 or more. The upper limit of the above peak area ratio (RD/RQ9) is not particularly limited, but may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.0 or less, in one embodiment, 2.8 or less, in one embodiment, 2.6 or less, in one embodiment, 2.4 or less, in one embodiment, 2.2 or less, or in one embodiment, 2.1 or less. That is, the above peak area ratio may be 0.3 or more, and, for example, may be within the range of 0.3 to 10.0, in one embodiment, 0.3 to 9.0, in one embodiment, 0.3 to 8.0, in one embodiment, 0.3 to 7.0, in one embodiment, 0.3 to 6.0, in one embodiment, 0.3 to 5.0, in one embodiment, 0.3 to 4.0, in one embodiment, 0.3 to 3.0, in one embodiment, 0.3 to 2.8, in one embodiment, 0.3 to 2.6, in one embodiment, 0.3 to 2.4, in one embodiment, 0.3 to 2.2, in one embodiment, 0.3 to 2.1, in one embodiment, 0.4 to 2.1, in one embodiment, 0.5 to 2.1, in one embodiment, 0.6 to 2.1, in one embodiment, 0.7 to 2.1, in one embodiment, 0.8 to 2.1, in one embodiment, 0.9 to 2.1, in one embodiment, 1.0 to 2.1, in one embodiment, 1.1 to 2.1, in one embodiment, 1.2 to 2.1, in one embodiment, 1.3 to 2.1, in one embodiment, 1.4 to 2.1, in one embodiment, 1.5 to 2.1, in one embodiment, 1.6 to 2.1, in one embodiment, 1.7 to 2.1, in one embodiment, 1.8 to 2.1, in one embodiment, 1.9 to 2.1, in one embodiment, 2.0 to 2.1, in one embodiment, 0.5 to 10.0, in one embodiment, 0.5 to 3.0, in one embodiment, 0.8 to 8.0, in one embodiment, 1.0 to 6.0, in one embodiment, 1.0 to 2.0, in one embodiment, 1.5 to 4.0, or in one embodiment, 2.0 to 3.0.

In one embodiment, the composition in solid form containing reduced coenzyme Q10 crystal, reduced demethoxy Q10, and reduced coenzyme Q9 can be separated from a solution by adding reduced coenzyme Q10 crystal as a seed crystal to a solution containing reduced coenzyme Q10, reduced demethoxy Q10, and reduced coenzyme Q9, and then precipitating the composition in solid form from the solution. That is, in the method according to an embodiment, the step of causing coexistence, in a composition, of the reduced coenzyme Q10 crystal with reduced demethoxy Q10 and reduced coenzyme Q9 may comprise adding reduced coenzyme Q10 crystal as a seed crystal to a solution containing reduced coenzyme Q10, reduced demethoxy Q10, and reduced coenzyme Q9, and precipitating the composition in solid form from the solution in which the seed crystal is added.

A solution containing reduced coenzyme Q10, reduced demethoxy Q10, and reduced coenzyme Q9 (which may hereinafter be referred to as “reduced coenzyme Q10 and the like”) may be a homogeneous solution state with dissolution of reduced coenzyme Q10 and the like in an organic solvent, or may be a slurry state with a part remaining without dissolution, however, a homogeneous solution state is preferred. In addition, the solution may contain various compounds in addition to the above reduced coenzyme Q10, reduced demethoxy Q10, and reduced coenzyme Q9, and examples include QH analogs such as reduced coenzyme Q11 and decaprenylphenol.

It is to be noted that, as for the reduced coenzyme Q10 for use in the above solution, there is no limitation as to whether it is in a crystal state or an amorphous state, and its crystal polymorphism is also not limited. Therefore, the use of conventionally known Form I type reduced coenzyme Q10 is also possible. Also, since it is possible to increase its purity in crystal precipitation, reduced coenzyme Q10 having analogs, or unpurified or crudely purified reduced coenzyme Q10 can be used. Furthermore, an extract solution of reduced coenzyme Q10 obtained by conventionally known methods, or a reaction solution that contains reduced coenzyme Q10 obtained from oxidized coenzyme Q10 by a known reduction method, as is, or, if necessary, after purification and/or solvent substitution, can be used as the solution containing reduced coenzyme Q10 and the like. For example, in a solution in which oxidized coenzyme Q10 and reduced coenzyme Q10 are present together, a solution obtained by subjecting only oxidized coenzyme Q10 to reduction treatment by a known reduction method, or, in a solution containing oxidized coenzyme Q10 and not containing reduced coenzyme Q10 nor components other than coenzyme Q10, a solution obtained by subjecting oxidized coenzyme Q10 to reduction treatment by a known reduction method, or, in a solution in which oxidized coenzyme Q10 and components other than coenzyme Q10 are present together, a solution obtained by subjecting only oxidized coenzyme Q10 to reduction treatment by a known reduction method, or the like, can be used as a solution containing reduced coenzyme Q10 and the like.

As a solvent for use in a solution containing reduced coenzyme Q10, reduced demethoxy Q10, and reduced coenzyme Q9, an organic solvent can be used, it is preferred to use at least one organic solvent selected from the group consisting of alcohol, hydrocarbon, fatty acid ester, and nitrogen compound.

As the alcohol, regardless of whether cyclic alcohols or acyclic alcohols and, further, regardless of whether saturated alcohols or unsaturated alcohols, it is not particularly limited, but, in general, saturated alcohols are preferably used. For example, typically, a monohydric alcohol having 1 or more carbon atoms, in one embodiment, having 2 or more carbon atoms, typically a monohydric alcohol having 20 or less carbon atoms, in one embodiment, having 12 or less carbon atoms, in one embodiment, having 6 or less carbon atoms, in one embodiment, having 5 or less carbon atoms, in one embodiment, having 4 or less carbon atoms, in one embodiment, having 3 or less carbon atoms is preferred, or typically, a dihydric alcohol having 2 or more carbon atoms, typically having 5 or less carbon atoms is preferred, or a trihydric alcohol having 3 carbon atoms is preferred. For example, a monohydric alcohol having 1 to 20 carbon atoms, 1 to 12 carbon atoms, particularly 1 to 6 carbon atoms, more particularly 1 to 5 carbon atoms, still more particularly 1 to 4 carbon atoms, even more particularly 1 to 3 carbon atoms, and still further having 2 to 3 carbon atoms is preferred, or a dihydric alcohol having 2 to 5 carbon atoms is preferred, or a trihydric alcohol having 3 carbon atoms is preferred. Among these, a monohydric alcohol of 1 to 5 carbon atoms is an alcohol having high compatibility with water and is preferably used in the case of use as a mixed solvent with water.

Examples of the monohydric alcohols can include methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol, 4-methylcyclohexanol, and the like.

The monohydric alcohol is preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, and cyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, and neopentyl alcohol, still more preferably methanol, ethanol, 1-propanol, 2-propanol, 1-butanol, 2-butanol, isobutyl alcohol, 2-methyl-1-butanol, and isopentyl alcohol, in particular, methanol, ethanol, 1-propanol, and 2-propanol, further preferably, ethanol, 1-propanol, and 2-propanol, and most preferably ethanol.

Examples of the dihydric alcohols include 1,2-ethanediol, 1,2-propanediol, 1,3-propanediol, 1,2-butanediol, 1,3-butanediol, 1,4-butanediol, 2,3-butanediol, 1,5-pentanediol, and the like. Preferably, the dihydric alcohol is 1,2-ethanediol, 1,2-propanediol, or 1,3-propanediol, most preferably, 1,2-ethanediol.

As a trihydric alcohol, glycerin or the like can preferably be used.

The hydrocarbon is not particularly limited, and examples thereof include an aliphatic hydrocarbon, an aromatic hydrocarbon, a halogenated hydrocarbon, and the like.

As the aliphatic hydrocarbon, regardless of whether cyclic aliphatic hydrocarbons or acyclic aliphatic hydrocarbons, and regardless of whether saturated aliphatic hydrocarbons or unsaturated aliphatic hydrocarbons, it is not particularly limited, but typically the aliphatic hydrocarbons having 3 or more carbon atoms, in one embodiment the aliphatic hydrocarbons having 5 or more carbon atoms, typically the aliphatic hydrocarbons having 20 or less carbon atoms, and in one embodiment the aliphatic hydrocarbons having 12 or less carbon atoms are used. The aliphatic hydrocarbon is not particularly limited regardless of whether cyclic aliphatic hydrocarbons or acyclic aliphatic hydrocarbons, and regardless of whether saturated aliphatic hydrocarbons or unsaturated aliphatic hydrocarbons, and typically the aliphatic hydrocarbons having 3 to 20 carbon atoms, preferably 5 to 12 carbon atoms, are used. Specific examples include propane, butane, isobutane, pentane, 2-methylbutane, cyclopentane, 2-pentene, hexane, 2-methylpentane, 2,2-dimethylbutane, 2,3-dimethylbutane, methylcyclopentane, cyclohexane, 1-hexene, cyclohexene, heptane, 2-methylhexane, 3-methylhexane, 2,3-dimethylpentane, 2,4-dimethylpentane, methylcyclohexane, 1-heptene, octane, 2,2,3-trimethylpentane, isooctane, ethylcyclohexane, 1-octene, nonane, 2,2,5-trimethylhexane, 1-nonene, decane, 1-decene, p-menthane, undecane, dodecane, and the like. Preferable examples are pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, nonane, 2,2,5-trimethylhexane, decane, dodecane, cyclopentane, methylcyclopentane, cyclohexane, methylcyclohexane, ethylcyclohexane, p-menthane, and the like. More preferable examples are pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, cyclopentane, methylcyclopentane, cyclohexane, methylcyclohexane, ethylcyclohexane, and the like, even more preferable examples are pentane, hexane, cyclohexane, methylcyclohexane, and the like, particularly preferable examples are heptane, hexane, methylcyclohexane, and most preferable examples are heptane and hexane.

The aromatic hydrocarbon is not particularly limited, but typically the aromatic hydrocarbons having 6 or more carbon atoms, in one embodiment, the aromatic hydrocarbons having 7 or more carbon atoms, typically the aromatic hydrocarbons having 20 or less carbon atoms, in one embodiment, the aromatic hydrocarbons having 12 or less carbon atoms, and in one embodiment the aromatic hydrocarbons having 10 or less carbon atoms are used. The aromatic hydrocarbon is not particularly limited, but typically the aromatic hydrocarbons having 6 to 20 carbon atoms, preferably 6 to 12 carbon atoms, more preferably 7 to 10 carbon atoms are used. Specific examples include benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene, styrene, and the like.

The halogenated hydrocarbon is not particularly limited regardless of whether cyclic halogenated hydrocarbons or acyclic halogenated hydrocarbons and regardless of whether saturated halogenated hydrocarbons or unsaturated halogenated hydrocarbons, but acyclic ones are preferably used. Chlorinated hydrocarbons and fluorinated hydrocarbons are more preferred, and chlorinated hydrocarbons are further preferred.

As the halogenated hydrocarbon, preferably the halogenated hydrocarbons having 1 or more carbon atoms, preferably the halogenated hydrocarbons having 6 or less carbon atoms, more preferably the halogenated hydrocarbons having 4 or less carbon atoms, and more preferably the halogenated hydrocarbons having 2 or less carbon atoms are used. Further, as the halogenated hydrocarbon, preferably the halogenated hydrocarbons having 1 to 6 carbon atoms, more preferably the halogenated hydrocarbons having 1 to 4 carbon atoms, and more preferably the halogenated hydrocarbons having 1 to 2 carbon atoms are used. Specific examples include dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2-tetrachloroethane, pentachloroethane, hexachloroethane, 1,1-dichloroethylene, 1,2-dichloroethylene, trichloroethylene, tetrachloroethylene, 1,2-dichloropropane, 1,2,3-trichloropropane, chlorobenzene, and 1,1,1,2-tetrafluoroethane, and the like.

The fatty acid esters are not particularly limited, and examples thereof include propionic acid esters (propionate), acetic acid esters (acetate), formic acid esters (formate), and the like. Acetic acid esters and formic acid esters are preferred, and acetic acid esters are more preferred.

The ester group is not particularly limited, and examples thereof include an alkyl ester having 1 or more carbon atoms, an alkyl ester having 8 or less carbon atoms, an aralkyl ester having 1 or more carbon atoms, and, typically, an aralkyl ester having 8 or less carbon atoms, preferably having 6 or less carbon atoms, more preferably having 4 or less carbon atoms, and the like. The ester group is not particularly limited, and examples thereof include an alkyl ester having 1 to 8 carbon atoms, an aralkyl ester having 1 to 8 carbon atoms, and the like. The ester group is preferably an alkyl ester having 1 to 6 carbon atoms, and more preferably an alkyl ester having 1 to 4 carbon atoms.

Examples of the propionic acid esters include methyl propionate, ethyl propionate, butyl propionate, isopentyl propionate, and the like.

Examples of the acetic acid esters include methyl acetate, ethyl acetate, propyl acetate, isopropyl acetate, butyl acetate, isobutyl acetate, sec-butyl acetate, pentyl acetate, isopentyl acetate, sec-hexyl acetate, cyclohexyl acetate, benzyl acetate, and the like. Preferred examples include methyl acetate, ethyl acetate, propyl acetate, isopropyl acetate, butyl acetate, isobutyl acetate, and the like, and most preferred is ethyl acetate.

Examples of the formic acid esters include methyl formate, ethyl formate, propyl formate, isopropyl formate, butyl formate, isobutyl formate, sec-butyl formate, pentyl formate, and the like.

As a nitrogen compound, for example, a nitrile can be used. As for the nitrile, the nitrile is not particularly limited regardless of whether cyclic nitriles or acyclic nitriles, and regardless of whether saturated nitriles or unsaturated nitriles, but saturated nitriles are preferably used. Typically, nitriles having 2 or more carbon atoms, typically nitriles having 20 or less carbon atoms, preferably having 12 or less carbon atoms, more preferably having 8 or less carbon atoms, are used. Typically, nitriles having 2 to 20 carbon atoms, preferably having 2 to 12 carbon atoms, more preferably having 2 to 8 carbon atoms, are used. Specific examples of the nitrile include acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chloropropionitrile, bromopropionitrile, methoxyacetonitrile, methyl cyanoacetate, ethyl cyanoacetate, tolunitrile, benzonitrile, chlorobenzonitrile, bromobenzonitrile, cyanobenzoic acid, nitrobenzonitrile, anisnitrile, phthalonitrile, bromotolunitrile, methyl cyanobenzoate, methoxybenzonitrile, acetylbenzonitrile, naphthonitrile, biphenylcarbonitrile, phenylpropionitrile, phenylbutyronitrile, methyl phenylacetonitrile, diphenylacetonitrile, naphthylacetonitrile, nitrophenylacetonitrile, chlorobenzyl cyanide, cyclopropanecarbonitrile, cyclohexanecarbonitrile, cycloheptanecarbonitrile, phenylcyclohexanecarbonitrile, and tolylcyclohexanecarbonitrile. Preferable examples are acetonitrile, propionitrile, succinonitrile, butyronitrile, isobutyronitrile, valeronitrile, methyl cyanoacetate, ethyl cyanoacetate, benzonitrile, tolunitrile, or chloropropionitrile, more preferable examples are acetonitrile, propionitrile, butyronitrile, or isobutyronitrile, and a most preferable example is acetonitrile.

Examples of the nitrogen compounds other than the above nitrile include nitromethane, triethylamine, pyridine, formamide, N-methylformamide, N,N-dimethylformamide, N,N-dimethylacetamide, N-methylpyrrolidone, and the like.

Among the above organic solvents, an alcohol or a hydrocarbon is preferred, and an alcohol is particularly preferred.

As the solvent for use in a solution that contains reduced coenzyme Q10 or the like, the ones exemplified above may be used alone, or in order to improve conditions that affect crystal precipitation conditions such as the solubility of each component of reduced coenzyme Q10 or the like, crystal precipitation concentration, yield, slurry physical properties, and crystal physical properties, two or more solvents may also be mixed in an appropriate proportion according to the properties of each solvent and used.

Further, as long as the use of at least one organic solvent selected from the group consisting of the above alcohol, hydrocarbon, fatty acid ester and nitrogen compound is made, the auxiliary use of organic solvents other than those is acceptable.

The solvent of the solution containing reduced coenzyme Q10 and the like may contain water in addition to the above organic solvent. The water content in the solvent, based on the total weight of the solvent, is preferably 0.01 wt % or more, more preferably 0.1 wt % or more, still more preferably 0.5 wt % or more, preferably 50 wt % or less, more preferably 40 wt % or less, still more preferably 34 wt % or less, and particularly preferably 15 wt % or less. The water content in the solvent, based on the total weight of the solvent, is preferably 0.01 to 50 wt %, more preferably 0.1 to 40 wt %, still more preferably 0.5 to 34 wt %, and particularly preferably 0.5 to 15 wt %. When the water content exceeds 50 wt %, there are cases where separation of the organic solvent and water occurs, the solubility of reduced coenzyme Q10 and the like in the solvent is greatly reduced, and it may be difficult to obtain the desired composition in solid form. It is preferred that the water in the solvent is homogeneously mixed with the organic solvent, and, from this viewpoint as well, alcohols such as ethanol or fatty acid esters such as ethyl acetate are preferably selected as the organic solvent.

The solvent is preferably a monohydric alcohol having 1 or more carbon atom that contains 15 wt % or less of water or contains no water, preferably a monohydric alcohol typically having 5 or less carbon atoms that contains 15 wt % or less of water or contains no water, preferably a monohydric alcohol having 1 or more carbon atom that contains 8 wt % or less of water or contains no water, and preferably a monohydric alcohol typically having 5 or less carbon atoms that contains 8 wt % or less of water or contains no water.

The solvent is preferably a monohydric alcohol having 1 to 5 carbon atoms that contains 15 wt % or less of water or contains no water, more preferably a monohydric alcohol having 1 to 5 carbon atoms that contains 8 wt % or less of water or contains no water, particularly preferably ethanol that contains 8 wt % or less of water or contains no water, and further preferably ethanol that contains 0.1 wt % or more and 8 wt % or less of water.

The concentrations of reduced coenzyme Q10, reduced demethoxy Q10, and reduced coenzyme Q9 in a solution can be appropriately adjusted according to the solvent for use and are not particularly limited. The concentration of the total of reduced coenzyme Q10, reduced demethoxy Q10, and reduced coenzyme Q9 in a solution is, with respect to the total weight of the solution, for example 80 wt % or less, preferably, 70 wt % or less, more preferably 60 wt % or less, further preferably 50 wt % or less, and particularly preferably 40 wt % or less. Also, from the standpoint of production efficiency, it is preferred that the concentration of reduced coenzyme Q10 and the like in the solution be adjusted to a relatively high concentration. The concentration of reduced coenzyme Q10 and the like in a solution is, with respect to the total weight of the solution, for example 1 wt % or more, preferably 5 wt % or more, and more preferably 10 wt % or more. The concentration of the total of reduced coenzyme Q10, reduced demethoxy Q10, and reduced coenzyme Q9 in a solution is, with respect to the total weight of the solution, for example 1 to 80 wt %, preferably 5 to 70 wt %, more preferably 10 to 60 wt %, further preferably 10 to 50 wt %, and particularly preferably 10 to 40 wt %.

The solution containing reduced coenzyme Q10 and the like is more preferably a supersaturated solution with dissolution at a concentration equal to or higher than the saturation concentration of each component of reduced coenzyme Q10 and the like, typically at a temperature of 32° C. or higher, typically at a temperature of 43° C. or lower. The solution containing reduced coenzyme Q10 and the like is more preferably a supersaturated solution with dissolution at a concentration equal to or higher than the saturation concentration of each component of reduced coenzyme Q10 and the like at a temperature within the range of 32 to 43° C. Such a supersaturated solution can be prepared by heating a raw material mixture that contains the solvent and the respective components of reduced coenzyme Q10 and the like to a temperature of 42° C. or higher, 45° C. or higher, more preferably 49° C. or higher, further preferably 70° C. or lower, and particularly preferably 55° C. or lower to effect dissolution of the respective components of reduced coenzyme Q10 and the like to obtain a solution, and by cooling the obtained solution after the heating to a temperature lower than the heating temperature and a temperature within the range of 32 to 43° C.

Particularly, in the case where the solvent is a monohydric alcohol having 1 to 5 carbon atoms (preferably ethanol) that contains 8 wt % or less of water or contains no water, the total concentration of reduced coenzyme Q10, reduced demethoxy Q10, and reduced coenzyme Q9 in the solution is preferably 5 wt % or more, particularly preferably 10 wt % or more, preferably 50 wt % or less, more preferably 40 wt % or less, more preferably 25 wt % or less, and particularly preferably 20 wt % or less. When the concentration is within the range above, a supersaturated solution of reduced coenzyme Q10 or the like is easy to prepare, and by addition of a seed crystal, precipitation of the crystal is easy.

The amount of reduced coenzyme Q10 crystal added as a seed crystal to the solution containing reduced coenzyme Q10, reduced demethoxy Q10 and reduced coenzyme Q9 (that is, seed crystal addition amount) is not particularly limited, but is, with respect to the amount of reduced coenzyme Q10 in the solution before addition of the seed crystal, preferably 0.001 wt % or more, more preferably 0.005 wt % or more, particularly preferably 0.01 wt % or more, preferably 30 wt % or less, more preferably 20 wt % or less, and particularly preferably 5 wt % or less. The amount of reduced coenzyme Q10 crystal added as a seed crystal to the solution containing reduced coenzyme Q10, reduced demethoxy Q10 and reduced coenzyme Q9 (that is, seed crystal addition amount) is not particularly limited, but is, with respect to the amount of reduced coenzyme Q10 in the solution before addition of the seed crystal, preferably 0.001 to 30 wt %, more preferably 0.005 to 20 wt %, and particularly preferably 0.01 to 5 wt %. Note that the reduced coenzyme Q10 crystal for use as a seed crystal is preferably one that includes the reduced coenzyme Q10 crystal of the Form I type, but it is also acceptable if the reduced coenzyme Q10 crystal of the Form II type or an amorphous form of the reduced coenzyme Q10 is included. A higher purity of the Form I type reduced coenzyme Q10 crystal is preferred, and, for example, the use of a seed crystal containing the Form I type reduced coenzyme Q10 crystal at a concentration of 50 wt % or more, preferably 75 wt % or more, more preferably 80 wt % or more, and still more preferably 90 wt % or more, is preferred.

The temperature of the solution at the time of addition of the seed crystal is preferably 25° C. or higher, and is preferably 43° C. or lower. It is preferred that the temperature of the solution at the time of addition of the seed crystal is within the range of 25 to 43° C. More preferably, the temperature of the solution at the time of addition of the seed crystal is 27° C. or higher, and, particularly preferably, is 29° C. or higher. Further, it is preferred that the temperature of the solution at the time of addition of the seed crystal be 41° C. or lower. If the temperature of the solution at the time of addition of the seed crystal exceeds 43° C., there are cases where dissolution of the added seed crystal occurs and precipitation of the crystal does not occur. Further, if a seed crystal is added to a solution at the temperature of less than 25° C., the proportion of the crystal in the composition in solid form obtained by precipitation may become low, or precipitation of the crystal may not occur.

After adding the seed crystal to the solution, when precipitating the composition in solid form from the solution (hereinafter, sometimes referred to as a “precipitation step”), the temperature of the solution is not particularly limited as long as the temperature of the solution at the time of addition of the seed crystal is within the above range, the temperature of the solution after adding the seed crystal may be maintained at preferably the temperature of −1° C. or higher, preferably the temperature of 41° C. or lower. After adding the seed crystal to the solution, when precipitating the composition in solid form from the solution (hereinafter, sometimes referred to as a “precipitation step”), the temperature of the solution is not particularly limited as long as the temperature of the solution at the time of addition of the seed crystal is within the above range, the temperature of the solution after adding the seed crystal may be maintained at preferably the temperature of −1° C. or higher, more preferably the temperature of with the range of −1 to 41° C. The time to maintain the solution in the temperature range is not particularly limited, but is, preferably 1 hour or more, more preferably 2 hours or more, even more preferably 4 hours or more, and particularly preferably 10 hours or more. The upper limit of the time to maintain the solution in the temperature range is not particularly limited, but a sufficient effect can be obtained in about 24 hours. That is, the time to maintain the solution in the temperature range can be within the range of, preferably 1 to 24 hours, more preferably 2 to 24 hours, even more preferably 4 to 24 hours, and particularly preferably 10 to 24 hours. In this case, for example, the solution may be maintained at a constant temperature in the range of −1 to 41° C. or, after addition of the seed crystal, the solution may be allowed to reach −1° C. while being gradually cooled. Further, throughout the precipitation step, it may be permissible to maintain the solution at a temperature of −1 to 41° C., or, after maintaining the solution at a temperature of −1 to 41° C. for, for example, a time of 1 hour or more, thereafter to perform cooling the solution.

The endpoint temperature of the precipitation step is not particularly limited, but, from the viewpoint of an increase in recovery amount, 35° C. or lower, more preferably 30° C. or lower, and particularly 25° C. or lower. The lower limit of the endpoint temperature of the precipitation step is the solidification temperature of the system, but the endpoint temperature is preferably −10° C. or higher, more preferably −1° C. or higher. Specifically, the endpoint temperature of the precipitation step may be set within the range of −10 to 35° C., preferably −1 to 30° C., more preferably −1 to 25° C.

In the precipitation step, it is preferred to control the precipitation amount of the composition in solid form per unit time and thereby control the formation of supersaturation. A preferred precipitation amount per unit time is, for example, equal to or less than a rate to precipitate about 50% of the total precipitation amount per unit time (that is, at most 50% amount/hour), and preferably equal to or less than a rate to precipitate 25% of the total precipitation amount per unit time (that is, at most 25% amount/hour).

In one embodiment, the precipitation step includes lowering the temperature of the mixed liquid over time, that is, a cooling crystallization. In cooling crystallization, the solution is cooled to lower the solubility in the liquid phase of each component such as reduced coenzyme Q10 and to promote precipitation. The cooling precipitation is preferably performed successively after the above step of maintaining the solution at a temperature of 25° C. or higher for a constant time to cause precipitation of the composition in solid form capable of precipitation at this temperature. Lowering the temperature of the solution over time includes continuously lowering the temperature of the solution with elapsed time, stepwise lowering the temperature of the solution, and combinations thereof. When lowering the temperature of the solution over time, the cooling rate is not particularly limited, but, for example, the cooling rate is such that the temperature drop per hour is 30° C. or lower, preferably 25° C. or lower, more preferably 20° C. or lower, more preferably 15° C. or lower, more preferably 10° C. or lower, more preferably 0.5° C. or higher, and more preferably 1° C. or higher. The cooling rate in the case of lowering the temperature of the solution over time may be a constant, or may be subject to change. In particular, if the cooling rate is such that, as the temperature of the solution decreases, the cooling rate increases continuously or stepwise, that is, so that the magnitude of the temperature decrease per hour becomes larger, efficient precipitation of reduced coenzyme Q10 and the like, whose residual amount in the liquid phase decreases as the temperature of the solution decreases, can be carried out. For example, until the temperature of the solution reaches 25° C., cool the solution at a rate at which the temperature decrease per hour is preferably 5° C. or lower, more preferably 3° C. or lower, and, in a stage in which the solution is further cooled to a temperature of less than 25° C., it is possible to cool the solution at a rate at which the temperature decrease per hour is preferably 6° C. or higher, more preferably 8° C. or higher. In one embodiment, the endpoint temperature that is reached by lowering the temperature of the solution over time is preferably 25° C. or lower, more preferably 20° C. or lower, more preferably 10° C. or lower, more preferably 7° C. or lower, and more preferably 5° C. or lower. The lower limit of the endpoint temperature is the solidification temperature of the system of the solution, and the endpoint temperature is preferably −10° C. or higher, preferably −7° C. or higher, preferably −5° C. or higher, and more preferably −1° C. or higher. For example, the endpoint temperature may be within the range of −10 to 25° C., preferably −10 to 20° C., more preferably −7 to 10° C., still more preferably −5 to 7° C., and particularly preferably −5 to 5° C.

It is preferred to carry out the precipitation step while causing forced flow of the solution after addition of the seed crystal. In order to suppress the formation of supersaturation and to carry out nucleation and crystal growth smoothly, or from the viewpoint of higher quality, as the agitation power requirement per unit volume, it is good to impart the solution a flowing of typically about 0.005 kW/m³ or more, preferably 0.01 kW/m³ or more, more preferably 0.015 kW/m³ or more, and still more preferably 0.03 kW/m³ or more. The above forced flow is typically imparted by rotation of a stirring blade, but if the above flowing is obtained, it is not necessarily required to use a stirring blade, and, for example, a method using circulation of a solution or the like may be utilized.

The step of causing coexistence of the reduced coenzyme Q10 crystal with reduced demethoxy Q10 and reduced coenzyme Q9 in a composition is not particularly limited, and, in addition to the above cooling crystallization, methods such as poor solvent crystallization and concentration crystallization can be utilized. It is preferred that it be cooling crystallization, or a method combining cooling crystallization with another crystallization method. Poor solvent crystallization is a method that, by mixing a poor solvent with the solution, lowers the solubility and causes precipitation of the composition in solid form. Here, the term poor solvent refers to a solvent in which either dissolution of reduced coenzyme Q10 hardly occurs or dissolution of reduced coenzyme Q10 does not occur at all. It is preferred that the poor solvent mutually dissolve with a solvent used in a solution that contains reduced coenzyme Q10 or the like.

As methods to mix a poor solvent with a solution, one may add the poor solvent to the solution, or one may add the solution to the poor solvent. Examples of other crystallization methods to be combined with cooling crystallization include, in addition to the above-described poor solvent crystallization, for example, concentration crystallization that causes precipitation of the crystal by concentrating a solution, and the like.

The composition in solid form obtained by the precipitation step can be recovered, for example, by a conventionally known method, through the steps of solid-liquid separation and drying. For example, for solid-liquid separation, the use of pressure filtration, filtration by centrifugation, and the like is possible. Additionally, the composition in solid form after drying can also be subjected to pulverization and/or classification as necessary.

By performing, under heating, the drying of the composition in solid form after the solid-liquid separation, it is possible to improve the content ratio of reduced coenzyme Q10 crystal in the composition in solid form. For this purpose, the drying temperature is preferably 33° C. or higher, more preferably 34° C. or higher, and still more preferably 35° C. or higher. Furthermore, the drying temperature is typically 46° C. or lower, preferably 45° C. or lower. For example, the drying temperature can be set within the range of preferably 33 to 46° C., more preferably 34 to 46° C., and still more preferably 35 to 45° C. When below 33° C., drying proceeds, but the content ratio of reduced coenzyme Q10 crystal in the composition in solid form hardly increases. In addition, when exceeding 46° C., during drying the composition in solid form may undergo melting. Also, the heating time in the case of performing drying under the above temperature conditions is not particularly limited, but is, preferably 4 hours or more, preferably 10 hours or more, and more preferably 20 hours or more.

Note that, in the precipitation step, when the content ratio of the reduced coenzyme Q10 crystal for the purpose has already been achieved, it is not limited to the above, and, for example, the drying may be carried out at 20° C. or higher, preferably 25° C. or higher, more preferably 30° C. or higher.

It is preferred that the solution preparation, the addition of the seed crystal, precipitation, recovery, described above, and subsequent processing and the like be carried out under a deoxygenated atmosphere. A deoxygenated atmosphere can be achieved by substitution of the atmosphere with an inert gas, by reduced pressure, by boiling, or by combining these. At least, substitution of the atmosphere with an inert gas, i.e., using an inert gas atmosphere, is preferred. Examples of the above inert gas include nitrogen gas, helium gas, argon gas, hydrogen gas, carbon dioxide gas, and the like, and nitrogen gas is preferred.

Whether the reduced coenzyme Q10 crystal of the Form I type or Form II type are contained in the obtained composition in solid form, and the content ratio thereof, can be determined, for example, by measuring with a differential scanning calorimeter (DSC).

The Form II type reduced coenzyme Q10 crystal, when measured by DSC at a heating rate of 1° C./min, shows an endothermic peak of 52±2° C., and the Form I type reduced coenzyme Q10 crystal, under the same conditions, shows an endothermic peak of 48±1° C. Whether the composition in solid form contains the reduced coenzyme Q10 crystal of the Form I type or Form II type can be determined by the presence or absence of the endothermic peak of 48±1° C. or 52±2° C., respectively. Furthermore, the content ratio of the Form I type to Form II type of the reduced coenzyme Q10 crystal in the composition in solid form can be determined by the ratio of the height of that endothermic peak thereof and/or the heats of absorption thereof.

In a case where the obtained composition in solid form contains Form I crystal, when Form II type reduced coenzyme Q10 crystal in an amount of 10 wt % with respect to the Form I crystal contained in the composition and 99.5% ethanol are added to the composition, and the mixture is stirred and then allowed to stand at 37° C. for a time of 6 hours, the proportion of the Form II crystal in the mixture, in a measurement by a differential scanning calorimeter (DSC), as the proportion (%) of the endothermic peak area of the Form II crystal to the total of the endothermic peak area of the Form I crystal and the endothermic peak area of the Form II crystal, is typically 25% or less, in one embodiment, 24% or less, in one embodiment, 23% or less, in one embodiment, 22% or less, in one embodiment, 21% or less, in one embodiment, 20% or less, in one embodiment, 19% or less, in one embodiment, 18% or less, in one embodiment, 17% or less, in one embodiment, 16% or less, in one embodiment, 15% or less, in one embodiment, 14% or less, in one embodiment, 13% or less, in one embodiment, 12% or less, in one embodiment, 11% or less, in one embodiment, 10% or less, in one embodiment, 9% or less, and in one embodiment, 8% or less. The lower limit value of the proportion is not limited, but the proportion is typically 1% or more.

In a case where the obtained composition in solid form contains Form I crystal, when Form II type reduced coenzyme Q10 crystal in an amount of 10 wt % with respect to the Form I crystal contained in the composition and 99.5% ethanol are added to the composition, and the mixture is stirred and then allowed to stand at 37° C. for a time of 6 hours, the fact that the proportion of Form II crystal in the mixture is small means that the transition rate from Form I crystal to Form II crystal of the reduced coenzyme Q10 composition according to one or more embodiments of the present invention is small, and therefore means that the polymorphic stability of the crystal of the reduced coenzyme Q10 composition according to one or more embodiments of the present invention is high. When the polymorphic stability of the crystal of the reduced coenzyme Q10 composition is high, quality change due to transition of crystal polymorphism is less likely to occur, and it is more preferred in terms of quality control.

When producing a composition in solid form through the separation of reduced coenzyme Q10, reduced demethoxy Q10, and reduced coenzyme Q9 from the solution containing reduced coenzyme Q10 and the like, for example, in accordance with each form such as powder, granules, tablets, hard capsules, or the like, it is possible to produce the composition by conventionally known steps.

For example, one can produce a composition in powder form by mixing the composition in solid form separated from the solution with emulsified starch and, if necessary, antioxidants, emulsifiers, binders, excipients and the like.

Further, one can produce a composition in granule form, for example, by mixing the composition in solid form separated from the solution with emulsified starch, a binder, and, as necessary, an antioxidant an emulsifier and the like, adding a liquid binder to the mixture, and going through steps of granulation, drying, classification, and pulverization.

Furthermore, one can produce a composition in tablet form by tableting the composition in the powder or granule form by a conventional method.

One can produce a composition in hard capsule form by filling a hard capsule with the composition in the powder or granule form by a conventional method.

The granulation method is not particularly limited, and, for example, may be appropriately selected from methods such as an extrusion granulation method, a stirring granulation method, a tumbling granulation method (a rotating granulation method), a dry granulation method, a compression granulation method, a powder bonding method, a fluidized bed granulation method, a coacervation method, a spray-drying method, a cold spray method, an evaporation method, and an in-liquid curing method. The granulation method is preferably carried out by a granulation method in which pressure is applied (for example, the extrusion granulation method, the stirring granulation method, the tumbling granulation method, the dry granulation method, the compression granulation method, the powder bonding method, and the like) or by the fluidized bed granulation method, more preferably by the extrusion granulation method, the stirring granulation method, or the fluidized bed granulation method, and particularly preferably by the extrusion granulation method or the stirring granulation method.

In the granulation process the reduced coenzyme Q10 crystal is not completely dissolved or melted, and is mixed and granulated while basically maintaining the crystal state.

The components other than the liquid binder of the mixture to be subjected to granulation may be in the form of a powder mixture. The characteristics of the respective components other than the liquid binder of the mixture are as described above.

Examples of the liquid binder include water, ethanol, and the like. Additionally, the above-described binder may be caused to undergo dissolution in a liquid such as water or ethanol and used as a liquid binder. As the liquid binder, water is particularly preferred.

Granulation can be carried out by appropriately setting conditions so that a composition in solid form having dimensions according to the application for the purpose is obtained.

It is preferred to further include a drying step of drying, after granulation, the composition in solid form and removing, by drying, volatile components derived from each raw material component and the liquid binder. Additionally, as necessary, by sieving or the like, it is possible to perform separation and recovery of a composition in solid form having a desired particle size.

One can produce the composition in tablet form by tableting a composition in powder form, or a composition in granule form obtained through steps such as granulation and drying, but the tableting method is not particularly limited. For example, it is possible to appropriately select from methods such as the direct compression method, the semi-direct compression method, a dry granulation compression method, and a wet granulation compression method. The tableting method is preferably performed by the direct compression method or the semi-direct compression method, and the direct compression method is more preferred.

The composition containing reduced coenzyme Q10 crystal and the like can also be added to general food and beverage. The food and beverage to which the composition containing reduced coenzyme Q10 crystal and the like is added is not particularly limited, and, for example, the composition containing reduced coenzyme Q10 crystal and the like can be appropriately added to and used in beverages such as milk beverages, soft drinks, sports drinks, nutritional drinks, beauty drinks, liquid nutritional preparations, and the like, confectionery such as chewing gum, chocolate, candies, jelly, cakes, biscuits, crackers, and the like, frozen desserts such as ice cream, ice confections, and the like, noodles such as udon, Chinese noodles, spaghetti, instant noodles, and the like, fish paste products such as kamaboko, chikuwa, hanpen, and the like, seasonings such as dressings, mayonnaise, sauces, and the like, bread, ham, rice porridge, cooked rice, soups, various retort foods, various frozen foods, and the like. A food or beverage containing the composition containing reduced coenzyme Q10 crystal and the like can be used in applications such as so-called health food, supplement, functional food, Food with Function Claims, nutritional supplement, Food for Specified Health Uses, Nutrient Function Food, nursing care food, Smile Care Food, mastication and swallowing aid food, thick flowing food, and food for patients and the like. Alternatively, it can also be used as other food forms. Furthermore, the composition containing reduced coenzyme Q10 crystal and the like according to one or more embodiments of the present invention can also be added to general pharmaceuticals. Furthermore, it can also be used for pet food, livestock feed, and the like.

### II a Method for Storing a Reduced Coenzyme Q10 Crystal

A method for storing a reduced coenzyme Q10 crystal according to one embodiment include the step of storing the reduced coenzyme Q10 crystal while causing coexistence of the reduced coenzyme Q10 crystal with reduced demethoxy Q10 and reduced coenzyme Q9 in a composition. The components and their contents in the composition, the form of the composition, the procedure for causing coexistence of the reduced coenzyme Q10 crystal with reduced demethoxy Q10 and reduced coenzyme Q9 in the composition, and the uses of the composition are the same as those in the above embodiments, so redundant explanations will be omitted.

The environment for storing the reduced coenzyme Q10 crystal (that is, environment for storing the compositions containing the reduced coenzyme Q10 crystal and the like) may have the controlled relative humidity. For example, the relative humidity may be controlled at 0% or more, and may be controlled at 60% or below, 50% or below, 40% or below, 30% or below, 20% or below, or 10% or below.

The environment for storing the reduced coenzyme Q10 crystal (that is, environment for storing the compositions containing the reduced coenzyme Q10 crystal and the like) may have the controlled relative humidity. For example, the relative humidity may be controlled at 0% to 60%, 0% to 50%, 0% to 40%, 0% to 30%, 0% to 20%, or 0% to 10%.

The environment for storing the reduced coenzyme Q10 crystal can be a deoxygenated atmosphere, and this can prevent or reduce the oxidation of reduced coenzyme Q10. The deoxygenated atmosphere can be achieved by substitution with an inert gas, by reduced pressure, or by combining these. Examples of the deoxygenated atmosphere include nitrogen atmosphere, helium atmosphere, argon atmosphere, hydrogen atmosphere, carbon dioxide atmosphere, and nitrogen atmosphere is preferred.

The temperature of the environment for storing the reduced coenzyme Q10 crystal is not particularly limited, but is typically 60° C. or lower, preferably 40° C. or lower, more preferably 30° C. or lower, and even more preferably 25° C. or lower, and typically 0° C. or higher, preferably 5° C. or higher, more preferably 10° C. or higher, and even more preferably 15° C. or higher, and typically room temperature. That is, the temperature of the environment for storing the reduced coenzyme Q10 crystal is typically 0 to 60° C., preferably 5 to 40° C., more preferably 10 to 30° C., even more preferably 15 to 25° C., and typically room temperature.

The environment for storing the reduced coenzyme Q10 crystal can be under normal pressure, elevated pressure, or reduced pressure and typically normal pressure.

The composition containing reduced coenzyme Q10 crystal and the like can be packaged in a packaging material. The packaging material is preferably substantially gas-tight. Examples of the packaging materials include films, jars and bottles, made of plastics such as polyethylene and polyethylene terephthalate (PET) or glass. Plastic films laminated with metal films such as aluminum or the like can also be used as the packaging materials.

The composition containing reduced coenzyme Q10 crystal and the like can be packaged together with moisture-proofing agent in a packaging material. Examples of the moisture-proofing agents include silica gel, calcium chloride, calcium oxide, and molecular sieves. Furthermore, the composition containing reduced coenzyme Q10 crystal and the like and the packaging material for packaging the composition may be placed in an outer packaging such as a steel drum, a fiber drum, or a cardboard box.

### <<Third Aspect (Disclosure of Japanese Patent Application No. 2025-058139)>>

The third aspect of one or more embodiments of the present invention relates to a method for improving an in vivo absorbability of reduced coenzyme Q10, a method for improving a digestive fluid solubility of reduced coenzyme Q10 and an oral composition of reduced coenzyme Q10.

The third aspect of one or more embodiments of the present invention aims to provide a method for improving an in vivo absorbability of reduced coenzyme Q10, a method for improving a digestive fluid solubility of reduced coenzyme Q10 and an oral composition of reduced coenzyme Q10.

The present inventors have found that, by allowing coexistence of reduced coenzyme Q10 and analogs having chemical structures similar to reduced coenzyme Q10 and by controlling the peak area ratio in a chromatogram among the plurality of analogs relative to each other to be within a specific range, thereby the in vivo absorbability of reduced coenzyme Q10 improves, and have completed the third aspect of one or more embodiments of the present invention.

The gist of a third aspect of one or more embodiments of the present invention is as follows.
(1) A method for improving an in vivo absorbability of reduced coenzyme Q10 comprising
   a step of causing coexistence of the reduced coenzyme Q10 with reduced demethoxy Q10 and reduced coenzyme Q9, wherein, in a chromatogram of a coexisting state obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 ml/min, and
   a detection wavelength: 190 nm,
an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more.
(2) The method according to the above (1),
   wherein, in the chromatogram, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 1.0 or more.
(3) The method according to the above (1),
   wherein, in the chromatogram, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 2.0 or more.
(4) The method according to any one of the above (1) to (3), further comprising causing coexistence with an antioxidant.
(5) The method according to any one of the above (1) to (4),
   wherein the reduced coenzyme Q10 is caused to coexist with the reduced demethoxy Q10 and the reduced coenzyme Q9 in the composition in solid form.
(6) A method for improving a digestive fluid solubility of reduced coenzyme Q10 comprising a step of causing coexistence of the reduced coenzyme Q10 with reduced demethoxy Q10 and reduced coenzyme Q9, wherein, in a chromatogram of a coexisting state obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 mL/min, and
   a detection wavelength: 190 nm,
an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more.
(7) The method according to the above (6),
   wherein, in the chromatogram, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 1.0 or more.
(8) The method according to the above (6),
   wherein, in the chromatogram, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 2.0 or more.
(9) The method according to any one of the above (6) to (8) above, further comprising causing coexistence with an antioxidant.
(10) The method according to any one of the above (6) to (9),

wherein the reduced coenzyme Q10 is caused to coexist with the reduced demethoxy Q10 and the reduced coenzyme Q9 in the composition in solid form.
(11) An oral composition containing reduced coenzyme Q10, reduced demethoxy Q10, and reduced coenzyme Q9, wherein, in a chromatogram of the oral composition obtained by high performance liquid chromatography under the following conditions:
   a column: a C18 column (150 mm in length and 4.6 mm in inner diameter),
   a mobile phase composition: CH₃OH:C₆H₁₄=9:1 (v/v),
   a mobile phase flow rate: 1 mL/min, and
   a detection wavelength: 190 nm,
an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more.
(12) The oral composition according to the above (11),
   wherein, in the chromatogram, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 1.0 or more.
(13) The oral composition according to the above (11),
   wherein, in the chromatogram, the area ratio (RD/RQ9) of the peak containing the reduced demethoxy Q10 (RD) having the relative retention time (RRT) of 0.88 to 0.94 to the peak containing the reduced coenzyme Q9 (RQ9) having the relative retention time (RRT) of 0.69 to 0.73 may be 2.0 or more.
(14) The oral composition according to any one of the above (11) to (13), further containing an antioxidant.
(15) The oral composition according to any one of the above (11) to (14),
   wherein the oral composition is in solid form.

The third aspect of one or more embodiments of the present invention provides a method for improving an in vivo absorbability of reduced coenzyme Q10, a method for improving a digestive fluid solubility of reduced coenzyme Q10 and an oral composition of reduced coenzyme Q10.

A method for improving an in vivo absorbability of reduced coenzyme Q10 and a method for improving a digestive fluid solubility of reduced coenzyme Q10, which are one aspect of one or more embodiments of the present invention (hereinafter sometimes simply referred to as “the method”), are characterized, in one embodiment, by the coexistence of reduced coenzyme Q10 with reduced demethoxy Q10 and reduced coenzyme Q9. Hereinafter, the state of coexistence of reduced coenzyme Q10 with reduced demethoxy Q10 and reduced coenzyme Q9 may simply be referred to as “coexisting state”. An oral composition of reduced coenzyme Q10 of one or more embodiments of the present invention, is characterized, in one embodiment, by containing reduced coenzyme Q10, reduced demethoxy Q10 and reduced coenzyme Q9.

In the present specification, the term “in vivo” means inside the body, inside a tissue, or inside a cell, of an organism. The organism is not particularly limited, but mammals, birds, fish, or the like are preferred. The above-described tissue is not particularly limited, and examples thereof include the skin, the digestive system, and the like. The digestive system is not particularly limited, and examples thereof include the digestive tract, the pancreas, the liver, the gallbladder, and the like. The digestive tract is not particularly limited, and examples thereof include the esophagus, stomach, duodenum, small intestine, and large intestine, and the like.

In the present specification, the term “in vivo absorption” means that the reduced coenzyme Q10 that is the subject is taken up in vivo. In one embodiment, “in vivo absorption” means that the reduced coenzyme Q10 that is the subject is taken up in a state in which it has been decomposed by a digestive enzyme or the like.

In the present specification, the term “improvement of in vivo absorbability” means that the reduced coenzyme Q10 that is the subject becomes more easily absorbed in vivo, as compared with reduced coenzyme Q10 alone.

In the present specification, the term “digestive fluid” means a generic term for liquids containing digestive enzymes secreted from digestive glands in the digestive system. The digestive fluid is not particularly limited, and examples thereof include saliva, gastric juice, bile, pancreatic juice, and intestinal juice and the like.

In the present specification, the term “the improvement in digestive fluid solubility” means that the reduced coenzyme Q10 that is the subject becomes more readily subject to dissolution in digestive fluid, as compared with reduced coenzyme Q10 alone. Digestive fluid solubility can be evaluated, for example, by the solubility with respect to digestive fluid.

In one embodiment, in vivo absorbability improves as digestive fluid solubility improves.

In the present specification, the term “coexistence” means that two or more substances are present simultaneously in the same system, for example, reduced coenzyme Q10 is present simultaneously with reduced demethoxy Q10 and reduced coenzyme Q9 within a specific region. Within the specific region is not particularly limited, and examples thereof include in a liquid, in an emulsion, in a solid, or in a suspension containing a liquid and a solid.

### <Coenzyme Q10>

The reduced coenzyme Q10 according to one or more embodiments of the present invention is the reduced coenzyme Q10 having the following chemical structure. Furthermore, in the method and the oral composition of one or more embodiments of the present invention, oxidized coenzyme Q10 having the following chemical structure may be caused to coexist or contained (hereinafter sometimes simply referred to as “containing”). In one embodiment, a main component of coenzyme Q10 in the coexisting state or the oral composition is reduced coenzyme Q10. Here, the main component means that, with respect to the total weight of coenzyme Q10 in the coexisting state or the oral composition, the coexisting content or content (hereinafter, “coexisting content or content” is sometimes simply referred to as “content”) of reduced coenzyme Q10 is, for example, 50 wt % or more, typically 60 wt % or more, preferably 70 wt % or more, more preferably 80 wt % or more, still more preferably 90 wt % or more, particularly preferably 95 wt % or more, and especially 98 wt % or more.

### [Chem. 11]

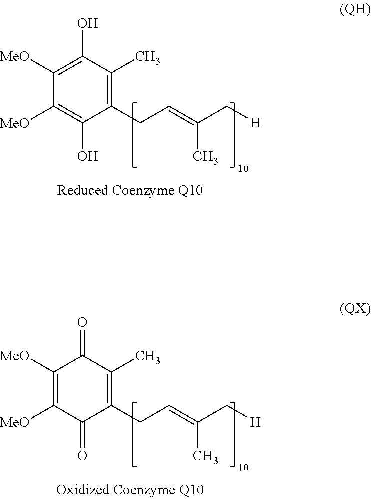

Reduced coenzyme Q10 can be obtained, for example, by known methods such as synthesis, fermentation, and extraction from a natural product, as well as by combining those with a reduction reaction as necessary. Preferably, it can be obtained by reducing oxidized coenzyme Q10, such as existing high-purity coenzyme Q10, or a mixture of oxidized coenzyme Q10 and reduced coenzyme Q10, using a general reducing agent, for example, sodium dithionite, sodium borohydride, ascorbic acids and the like.

In one or more embodiments of the present invention, the reduced coenzyme Q10 in the coexisting state or the oral composition is caused to coexist or is contained in solid form. In this case, the reduced coenzyme Q10 may be caused to coexist or be contained in the state of an amorphous solid, may be caused to coexist or be contained in the state of a crystal, or may be caused to coexist or be contained in the state of a crystalline solid. In the present specification, the term “amorphous solid” means a solid containing an amorphous component that does not have a crystal structure. An amorphous solid may include a portion having a crystal structure, but in order to be an amorphous solid, the amorphous component of QH is, with respect to the total weight of QH, preferably 80 wt % or more, more preferably 90 wt % or more, more preferably 95 wt % or more, and more preferably 98 wt % or more. In the present specification, the term “crystalline solid” means a solid that contains therein, together with a portion having a crystal structure, an amorphous component that does not have a crystal structure. Specifically, in order to be a crystalline solid, the crystal of QH is, with respect to the total weight of QH, preferably 80 wt % or more, more preferably 90 wt % or more, even more preferably 95 wt % or more, and more preferably 98 wt % or more. In one embodiment, the reduced coenzyme Q10 in the coexisting state or the oral composition is preferably caused to coexist or is contained in the state of crystal or crystalline solid. Specifically, the crystal of QH is preferably, with respect to the total weight of QH, preferably 80 wt % or more, more preferably 90 wt % or more, more preferably 95 wt % or more, and more preferably 98 wt % or more. Note that the degree of crystallization of each component such as reduced coenzyme Q10 and the like in the coexisting state or the oral composition can be calculated, in accordance with the following formula, from the theoretical melting heat obtained from the content of each component such as reduced coenzyme Q10 and the like in the coexisting state or the oral composition and from the measured melting heat data obtained by measuring the crystal melting heat by DSC analysis.

Degree of crystallization (%)=(measured melting enthalpy/theoretical melting enthalpy)×100

In the reduced coenzyme Q10, there are two crystal polymorphs, the Form I type and the Form II type, and both are applicable. Specifically, the crystal form of reduced coenzyme Q10 having a melting point around 48° C. and showing characteristic peaks at diffraction angles (2θ±0.2°) of 3.1°, 18.7°, 19.0°, 20.2°, and 23.0° in powder X-ray (Cu-Kα) diffraction is a Form I crystal, and the crystal form of reduced coenzyme Q10 having a melting point around 52° C. and showing characteristic peaks at diffraction angles (2θ±0.2°) of 11.5°, 18.2°, 19.3°, 22.3°, 23.0°, and 33.3° in powder X-ray (Cu-Kα) diffraction is a Form II crystal.

In one or more embodiments of the present invention, the reduced coenzyme Q10 in the coexisting state or the oral composition may be caused to coexist or be contained in a state of a liquid.

In one or more embodiments of the present invention, the reduced coenzyme Q10 in the coexisting state or the oral composition may be caused to coexist or be contained in a state of an emulsion.

In one or more embodiments of the present invention, the reduced coenzyme Q10 in the coexisting state or the oral composition may be caused to coexist or be contained in a state of a suspension containing a solid and a liquid.

In a method for improving an in vivo absorbability of reduced coenzyme Q10 and an oral composition of reduced coenzyme Q10 according to one or more embodiments of the present invention, the content of reduced coenzyme Q10 in the coexisting state or the oral composition (weight of reduced coenzyme Q10/total weight of total components of coexisting state or total weight of oral composition) is not particularly limited, but is typically about 0.1 wt % or more, preferably about 0.5 wt % or more, more preferably about 1 wt % or more, more preferably about 2 wt % or more, and more preferably about 5 wt % or more. The upper limit is not particularly limited, but is 50 wt % or less, preferably 49 wt % or less, more preferably 48 wt % or less, more preferably 47 wt % or less, and more preferably 46 wt % or less. That is, the content of reduced coenzyme Q10 in the coexisting state or the oral composition can be within the range of 0.1 to 50 wt %, preferably 0.5 to 49 wt %, more preferably 1 to 48 wt %, more preferably 2 to 47 wt %, and more preferably 5 to 46 wt %.

In a method for improving an in vivo absorbability of reduced coenzyme Q10 and an oral composition of reduced coenzyme Q10 according to one or more embodiments of the present invention, the content of reduced coenzyme Q10 in the coexisting state or the oral composition (weight of reduced coenzyme Q10/total weight of total components of coexisting state or total weight of oral composition) is typically 51 wt % or more, preferably 56 wt % or more, more preferably 61 wt % or more, more preferably 66 wt % or more, and more preferably 71 wt % or more. The upper limit is not particularly limited, but is 99 wt % or less, preferably 98 wt % or less, more preferably 97 wt % or less, more preferably 96 wt % or less, and more preferably 95 wt % or less. That is, the content of reduced coenzyme Q10 in the coexisting state or the oral composition can be within the range of typically 51 to 99 wt %, preferably 56 to 98 wt %, more preferably 61 to 97 wt %, more preferably 66 to 96 wt %, and more preferably 71 to 95 wt %.

In a method for improving an in vivo absorbability of reduced coenzyme Q10 and an oral composition of reduced coenzyme Q10 according to one or more embodiments of the present invention, in addition to the above reduced coenzyme Q10, reduced demethoxy Q10 (which may hereinafter be referred to as “RD”) and reduced coenzyme Q9 (which may hereinafter be referred to as “RQ9”), having the following chemical structure, are caused to coexist or are contained.

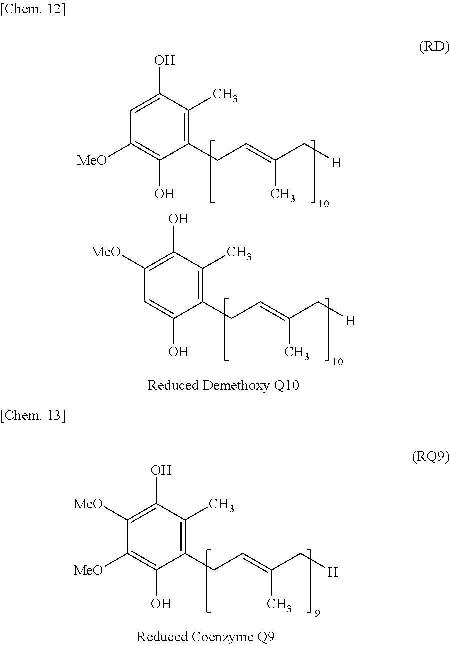

Reduced demethoxy Q10 (RD) corresponds to a compound in which the methoxy group in QH is missing, and reduced coenzyme Q9 (RQ9) corresponds to a compound in which the isoprene chain in QH is shorter by one unit. Note that, in the present specification, “reduced demethoxy Q10 (RD)” also includes analogs of reduced demethoxy Q10. Examples of the analogs of the reduced demethoxy Q10 include compounds in which the isoprene chain of the reduced demethoxy Q10 has undergone cyclization at one site or double-bond formation at one site, compounds in which cyclization has occurred at one site or at two or more sites of the isoprene chain of QH, and compounds in which the formation of a double bond has occurred at one site or at two or more sites of the isoprene chain of QH. These RD and RQ9, as with QH, can be obtained by known methods such as synthesis, fermentation, and extraction from natural products, as well as by combining those with a reduction reaction, as necessary. Preferably, RD and RQ9, that are generated together as by-products when reduced coenzyme Q10 is produced by reducing a composition containing existing oxidized coenzyme Q10 with a general reducing agent, for example, sodium dithionite, sodium borohydride, ascorbic acids, or the like, may be used as they are.

In one or more embodiments of the present invention, RD and RQ9 in the coexisting state or in the oral composition are caused to coexist or are contained in solid form. In this case, RD and RQ9 may be caused to coexist or be contained in a state of an amorphous solid, may be caused to coexist or be contained in a state of a crystal, or may be caused to coexist or be contained in a state of a crystalline solid. In one embodiment, RD and RQ9 in the coexisting state or in the oral composition preferably are caused to coexist or are contained in the state of a crystal or crystalline solid, and specifically, the crystal is, with respect to the total weight of RD and RQ9, preferably 80 wt % or more, more preferably 90 wt % or more, more preferably 95 wt % or more, more preferably 98 wt % or more.

In one or more embodiments of the present invention, RD and RQ9 in the coexisting state or in the oral composition may be caused to coexist or be contained in a state of a liquid.

In one or more embodiments of the present invention, RD and RQ9 in the coexisting state or in the oral composition may be caused to coexist or be contained in a state of a suspension containing a solid and a liquid.

In one or more embodiments of the present invention, RD and RQ9 in the coexisting state or in the oral composition may be caused to coexist or be contained in a state of an emulsion.

The total content of RD and RQ9 (RD and RQ9 total weight/total weight of total components of coexisting state or total weight of oral composition) in the coexisting state or in the oral composition is not particularly limited, but is typically 0.001 wt % or more, preferably 0.005 wt % or more, more preferably 0.01 wt % or more, more preferably 0.015 wt % or more, and more preferably 0.02 wt % or more. The upper limit is not particularly limited, but is 10 wt % or less, preferably 8 wt % or less, more preferably 6 wt % or less, more preferably 4 wt % or less, and more preferably 2 wt % or less. That is, in the coexisting state or in the oral composition, the total content of RD and RQ9 (RD and RQ9 total weight/total weight of total components of coexisting state or total weight of oral composition) can typically be in the range of 0.001 to 10 wt %, preferably 0.005 to 8 wt %, more preferably 0.01 to 6 wt %, more preferably 0.015 to 4 wt %, and more preferably 0.02 to 2 wt %. The content of RD (RD weight/total weight of total components of coexisting state or total weight of oral composition) in the coexisting state or in the oral composition is within the range of preferably 0.0005 to 5 wt %, more preferably 0.001 to 3 wt %, and the content of RQ9 (RQ9 weight/total weight of total components of coexisting state or total weight of oral composition) is within the range of preferably 0.002 to 4 wt %, more preferably 0.005 to 2 wt %.

The content of RD (RD weight/total weight of total components of coexisting state or total weight of oral composition) in the coexisting state or in the oral composition is, for example, 0.0005 wt % or more, in one embodiment, 0.001 wt % or more and, for example, 5 wt % or less, in one embodiment, 3 wt % or less, and, for example, within the range of 0.0005 wt % to 5 wt %, in one embodiment, 0.001 wt % to 3 wt %, and the content of RQ9 (RQ9 weight/total weight of total components of coexisting state or total weight of oral composition) is, for example, 0.002 wt % or more, in one embodiment, 0.005 wt % or more, and, for example, 4 wt % or less, in one embodiment, 2 wt % or less, and, for example, within the range of 0.002 wt % to 4 wt %, in one embodiment, 0.005 wt % to 2 wt %.

And, in the method for improving an in vivo absorbability of reduced coenzyme Q10 and an oral composition of the reduced coenzyme Q10 composition according to one or more embodiments of the present invention, by adjusting a peak area ratio in the chromatogram of RD to RQ9 in the coexisting state or in the oral composition within a specific range, although the reason is not clear, it has become clear that the in vivo absorbability, particularly the digestive fluid solubility, for example the intestinal fluid solubility, of reduced coenzyme Q10 (QH) has been improved. Specifically, in the chromatogram of the coexisting state or in the oral composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more. The above peak area ratio (RD/RQ9) may be 0.5 or more, 0.8 or more, 1.0 or more, 1.5 or more, or 2.0 or more. The upper limit of the above peak area ratio (RD/RQ9) is not particularly limited, but may be 10.0 or less, 8.0 or less, 6.0 or less, 4.0 or less, or 3.0 or less. That is, the above peak area ratio may be within the range of 0.3 to 10.0, 0.5 to 10.0, 0.5 to 3.0, 0.8 to 8.0, 1.0 to 6.0, 1.0 to 2.0, 1.5 to 4.0, or 2.0 to 3.0.

In another embodiment, specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be 0.3 or more. The above peak area ratio (RD/RQ9) may be, for example, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, or in one embodiment, 2.0 or more. The upper limit of the above peak area ratio (RD/RQ9) is not particularly limited, but may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.0 or less, in one embodiment, 2.8 or less, in one embodiment, 2.6 or less, in one embodiment, 2.4 or less, in one embodiment, 2.2 or less, or in one embodiment, 2.1 or less. That is, the above peak area ratio may be 0.3 or more, and, for example, may be within the range of 0.3 to 10.0, in one embodiment, 0.3 to 9.0, in one embodiment, 0.3 to 8.0, in one embodiment, 0.3 to 7.0, in one embodiment, 0.3 to 6.0, in one embodiment, 0.3 to 5.0, in one embodiment, 0.3 to 4.0, in one embodiment, 0.3 to 3.0, in one embodiment, 0.3 to 2.8, in one embodiment, 0.3 to 2.6, in one embodiment, 0.3 to 2.4, in one embodiment, 0.3 to 2.2, in one embodiment, 0.3 to 2.1, in one embodiment, 0.4 to 2.1, in one embodiment, 0.5 to 2.1, in one embodiment, 0.6 to 2.1, in one embodiment, 0.7 to 2.1, in one embodiment, 0.8 to 2.1, in one embodiment, 0.9 to 2.1, in one embodiment, 1.0 to 2.1, in one embodiment, 1.1 to 2.1, in one embodiment, 1.2 to 2.1, in one embodiment, 1.3 to 2.1, in one embodiment, 1.4 to 2.1, in one embodiment, 1.5 to 2.1, in one embodiment, 1.6 to 2.1, in one embodiment, 1.7 to 2.1, in one embodiment, 1.8 to 2.1, in one embodiment, 1.9 to 2.1, in one embodiment, 2.0 to 2.1, in one embodiment, 0.5 to 10.0, in one embodiment, 0.5 to 3.0, in one embodiment, 0.8 to 8.0, in one embodiment, 1.0 to 6.0, in one embodiment, 1.0 to 2.0, in one embodiment, 1.5 to 4.0, or in one embodiment, 2.0 to 3.0.

Here, the peak area of each compound in the chromatogram can be calculated by an automatic integration method (in which an integrator automatically calculates from the signal intensity of the detector). In addition, the identification of each peak is performed by mass spectrometry. Furthermore, the peak area of each compound in the chromatogram can be appropriately changed by adjusting the content of each compound in the composition.

In the present embodiment, in the chromatogram obtained by high performance liquid chromatography under the above conditions, the area of the peak containing each of reduced coenzyme Q9 (RQ9), reduced demethoxy Q10 (RD), reduced coenzyme Q10 (QH), decaprenylphenol and reduced coenzyme Q11 (RQ11) to be described later, and oxidized coenzyme Q10 (QX) refers to the area of the peak detected at the retention time (RT) shown in the table below, or at the relative retention time (RRT) based on the retention time (RT, 14.72 min in Table 3) of the peak top of QH.

TABLE 3

| | Reduced | Reduced | Reduced |
|---|---|---|---|
| | Coenzyme Q9 | Demethoxy Q10 | Coenzyme Q10 |
| | (RQ9) | (RD) | (QH) |
| RT(min) | 10.10 to 10.75 | 12.90 to 13.90 | 14.00 to 15.75 |
| RRT | 0.69 to 0.73 | 0.88 to 0.94 | 0.95 to 1.07 |

| | | Reduced | Oxidized |
|---|---|---|---|
| | | Coenzyme Q11 | Coenzyme Q10 |
| | Decaprenylphenol | (RQ11) | (QX) |
| RT(min) | 19.75 to 20.7 | 21.30 to 22.0 | 24.75 to 26.25 |
| RRT | 1.34 to 1.41 | 1.45 to 1.49 | 1.68 to 1.78 |

In the present embodiment, in the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but, for example, may be 0.0005 or more, in one embodiment, 0.001 or more, in one embodiment, 0.002 or more, in one embodiment, 0.003 or more, in one embodiment, 0.004 or more, in one embodiment, 0.005 or more, and in one embodiment, 0.01 or less, in one embodiment, 0.008 or less, in one embodiment, 0.006 or less.

In the present embodiment, in the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but is within the range of preferably 0.0005 to 0.01, more preferably 0.001 to 0.008, more preferably 0.002 to 0.008, and more preferably 0.004 to 0.006.

In the present embodiment, in the chromatogram obtained by high performance liquid chromatography under the above conditions, the area ratio (RD/QH) of the peak containing reduced demethoxy Q10 (RD) to the peak containing reduced coenzyme Q10 (QH) is not particularly limited, but, for example, is within the range of 0.0005 to 0.01, in one embodiment, 0.001 to 0.008, in one embodiment, 0.001 to 0.006, in one embodiment, 0.002 to 0.006, in one embodiment, 0.003 to 0.006, in one embodiment, 0.004 to 0.006, in one embodiment, 0.005 to 0.006, and in one embodiment 0.002 to 0.008.

In the present embodiment, in the chromatogram obtained by high performance liquid chromatography under the above conditions, the area ratio (RQ9/QH) of the peak containing reduced coenzyme Q9 (RQ9) to the peak containing QH is not particularly limited, but, for example, may be 0.001 or more, in one embodiment, may be 0.002 or more, in one embodiment, may be 0.01 or less, in one embodiment, may be 0.008 or less, in one embodiment, may be 0.007 or less, in one embodiment, may be 0.006 or less, in one embodiment, may be 0.005 or less, in one embodiment, may be 0.004 or less, and in one embodiment may be 0.003 or less.

In the present embodiment, in the chromatogram obtained by high performance liquid chromatography under the above conditions, the area ratio (RQ9/QH) of the peak containing reduced coenzyme Q9 (RQ9) to the peak containing QH is not particularly limited, but is within the range of preferably 0.001 to 0.01, more preferably 0.001 to 0.006, more preferably 0.001 to 0.005, and more preferably 0.002 to 0.005.

In the present embodiment, in the chromatogram obtained by high performance liquid chromatography under the above conditions, the area ratio (RQ9/QH) of the peak containing reduced coenzyme Q9 (RQ9) to the peak containing QH is not particularly limited, but, for example, is within the range of 0.001 to 0.01, in one embodiment, 0.001 to 0.008, in one embodiment, 0.001 to 0.007, in one embodiment, 0.001 to 0.006, in one embodiment, 0.001 to 0.005, in one embodiment, 0.001 to 0.004, in one embodiment, 0.001 to 0.003, in one embodiment, 0.002 to 0.003, in one embodiment, 0.002 to 0.005.

In the present embodiment, in the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but, for example, may be 0.001 or more, in one embodiment, may be 0.005 or more, in one embodiment, may be 0.007 or more, in one embodiment, may be 0.008 or more, in one embodiment, may be 0.009 or more, in one embodiment, may be 0.01 or more, and in one embodiment may be 0.03 or less, in one embodiment, may be 0.02 or less, in one embodiment, may be 0.015 or less, in one embodiment, may be 0.012 or less, and in one embodiment may be 0.01 or less.

In the present embodiment, in the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but is within the range of preferably 0.001 to 0.03, more preferably 0.005 to 0.02, still more preferably 0.007 to 0.015, and particularly preferably 0.007 to 0.01.

In the present embodiment, in the chromatogram obtained by high performance liquid chromatography under the above conditions, the ratio ((RD+RQ9)/QH) of the total area of the peak containing reduced demethoxy Q10 (RD) and the peak containing reduced coenzyme Q9 (RQ9) to the area of the peak containing reduced coenzyme Q10 (QH) is not particularly limited, but, for example, is within the range of 0.001 to 0.03, in one embodiment, 0.005 to 0.03, in one embodiment, 0.007 to 0.03, in one embodiment, 0.007 to 0.02, in one embodiment, 0.007 to 0.015, in one embodiment, 0.007 to 0.012, in one embodiment, 0.008 to 0.012, in one embodiment, 0.009 to 0.012, in one embodiment, 0.01 to 0.012, in one embodiment, 0.005 to 0.02, in one embodiment, 0.007 to 0.01, in one embodiment, 0.008 to 0.01, and in one embodiment, 0.009 to 0.01.

Furthermore, in the method for improving an in vivo absorbability of reduced coenzyme Q10 and an oral composition of the reduced coenzyme Q10 of the present embodiment, in addition to the above RD and RQ9, as necessary, a QH analog may be caused to coexist or be contained. Examples of the QH analogs, including decaprenylphenol and reduced coenzyme Q11 (hereinafter may be referred to as “RQ11”) as shown below, can be given.

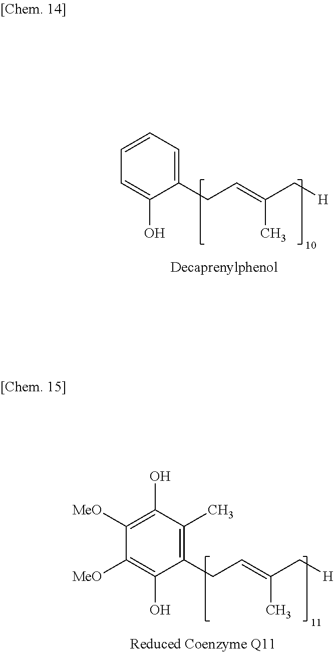

QH analogs such as the above decaprenylphenol and RQ11 can, similarly to QH, be obtained by known methods such as synthesis, fermentation, and extraction from natural products, as well as by combining those with a reduction reaction as needed. Preferably, QH analogs, such as decaprenylphenol and RQ11, that are generated together as by-products when reduced coenzyme Q10 is produced by reducing a composition containing existing oxidized coenzyme Q10 with a general reducing agent, for example, sodium dithionite, sodium borohydride, ascorbic acids, or the like, may be used as they are.

In the method for improving an in vivo absorbability of reduced coenzyme Q10 or an oral composition of the reduced coenzyme Q10, the total content of QH analog (RD, RQ11, RQ9 and decaprenylphenol) (total weight of QH analogs/total weight of total components of coexisting state or total weight of oral composition) is not particularly limited, but is typically 8 wt % or less, preferably 7 wt % or less, more preferably 6 wt % or less, more preferably 5 wt % or less, more preferably 4 wt % or less, and is typically 0.006 wt % or more, preferably 0.008 wt % or more, more preferably 0.01 wt % or more. In the method for improving an in vivo absorbability of reduced coenzyme Q10 or an oral composition of the reduced coenzyme Q10, the total content of QH analogs (RD, RQ11, RQ9, and decaprenylphenol) is typically 0.006 to 8 wt %, preferably 0.006 to 7 wt %, more preferably 0.008 to 6 wt %, still more preferably 0.008 to 5 wt %, and particularly preferably 0.01 to 4 wt %.

In the method for improving an in vivo absorbability of reduced coenzyme Q10 or an oral composition of the reduced coenzyme Q10, the content of decaprenylphenol (decaprenylphenol weight/total weight of total components of coexisting state or total weight of oral composition) in the reduced coenzyme Q10 composition is, in one embodiment, 0.001 wt % or more, in one embodiment, 0.002 wt % or more, in one embodiment, 3 wt % or less, in one embodiment, 2 wt % or less, and the content of RQ11 (RQ11 weight/total weight of total components of coexisting state or total weight of oral composition) is, in one embodiment, 0.005 wt % or more, in one embodiment, 0.01 wt % or more, in one embodiment, 5 wt % or less, and in one embodiment, 3 wt % or less.

In the method for improving an in vivo absorbability of reduced coenzyme Q10 or an oral composition of the reduced coenzyme Q10, the content of decaprenylphenol (decaprenylphenol weight/total weight of total components of coexisting state or total weight of oral composition) in the reduced coenzyme Q10 composition is within the range of preferably 0.001 to 3 wt %, more preferably 0.002 to 2 wt %, and the content of RQ11 (RQ11 weight/total weight of total components of coexisting state or total weight of oral composition) is within the range of preferably 0.005 to 5 wt %, more preferably 0.01 to 3 wt %.

In the present embodiment, in the chromatogram obtained by high performance liquid chromatography under the above conditions, the area ratio (decaprenylphenol/QH) of the peak containing decaprenylphenol to the peak containing reduced coenzyme Q10 (QH) is not particularly limited, but, for example, may be 0.001 or more, in one embodiment, 0.002 or more, in one embodiment, 0.003 or more, in one embodiment, 0.004 or more, in one embodiment, 0.005 or more, in one embodiment, 0.006 or more, in one embodiment, 0.007 or more, in one embodiment, 0.008 or more, in one embodiment, 0.009 or more, in one embodiment, 0.05 or less, in one embodiment, 0.03 or less, and in one embodiment, 0.01 or less.

In the present embodiment, in the chromatogram obtained by high performance liquid chromatography under the above conditions, the area ratio (decaprenylphenol/QH) of the peak containing decaprenylphenol to the peak containing reduced coenzyme Q10 (QH) is not particularly limited, but is within the range of preferably 0.001 to 0.05, more preferably 0.003 to 0.03, and more preferably 0.005 to 0.01.

In the present embodiment, in the chromatogram obtained by high performance liquid chromatography under the above conditions, the area ratio (decaprenylphenol/QH) of the peak containing decaprenylphenol to the peak containing QH is not particularly limited, but is, for example, within the range of 0.001 to 0.05, in one embodiment, 0.001 to 0.03, in one embodiment, 0.001 to 0.01, in one embodiment, 0.002 to 0.01, in one embodiment, 0.003 to 0.01, in one embodiment, 0.004 to 0.01, in one embodiment, 0.005 to 0.01, in one embodiment, 0.006 to 0.01, in one embodiment, 0.007 to 0.01, in one embodiment, 0.008 to 0.01, in one embodiment, 0.009 to 0.01, and in one embodiment, 0.003 to 0.03.

In the present embodiment, in the chromatogram obtained by high performance liquid chromatography under the above conditions, the area ratio (RQ11/QH) of the peak containing RQ11 to the peak containing QH is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.01 or more, in one embodiment, 0.015 or more, in one embodiment, 0.016 or more, in one embodiment, 0.017 or more, in one embodiment, 0.018 or more, in one embodiment, 0.019 or more, in one embodiment, 0.02 or more, in one embodiment, 0.05 or less, in one embodiment, 0.04 or less, in one embodiment, 0.03 or less, in one embodiment, 0.025 or less, and in one embodiment, 0.02 or less.

In the present embodiment, in the chromatogram obtained by high performance liquid chromatography under the above conditions, the area ratio (RQ11/QH) of the peak containing RQ11 to the peak containing QH is not particularly limited, but is within the range of preferably 0.001 to 0.05, more preferably 0.005 to 0.04, and even more preferably 0.01 to 0.03.

In the present embodiment, in the chromatogram obtained by high performance liquid chromatography under the above conditions, the area ratio (RQ11/QH) of the peak containing RQ11 to the peak containing QH is not particularly limited, but, for example, is within the range of 0.001 to 0.05, in one embodiment, 0.005 to 0.05, in one embodiment, 0.006 to 0.05, in one embodiment, 0.006 to 0.04, in one embodiment, 0.006 to 0.03, in one embodiment, 0.006 to 0.025, in one embodiment, 0.007 to 0.025, in one embodiment, 0.008 to 0.025, in one embodiment, 0.009 to 0.025, in one embodiment, 0.01 to 0.025, in one embodiment, 0.015 to 0.025, in one embodiment, 0.016 to 0.025, in one embodiment, 0.017 to 0.025, in one embodiment, 0.018 to 0.025, in one embodiment, 0.019 to 0.025, in one embodiment, 0.02 to 0.025, in one embodiment, 0.016 to 0.02, in one embodiment, 0.005 to 0.04, and in one embodiment 0.01 to 0.03.

In the present embodiment, in the chromatogram obtained by high performance liquid chromatography under the above conditions, the area ratio (decaprenylphenol/RQ9) of the peak containing decaprenylphenol to the peak containing RQ9 is not particularly limited, but may be, for example, 0.1 or more, in one embodiment, 0.2 or more, in one embodiment, 0.3 or more, in one embodiment, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.2 or more, in one embodiment, 1.4 or more, in one embodiment, 1.6 or more, in one embodiment, 1.8 or more, in one embodiment, 2.0 or more, in one embodiment, 2.2 or more, in one embodiment, 2.3 or more, in one embodiment, 2.4 or more, in one embodiment, 2.6 or more, in one embodiment, 2.8 or more, in one embodiment, 3.0 or more, in one embodiment, 3.2 or more, in one embodiment, 3.4 or more, or in one embodiment, 3.6 or more, and the upper limit may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.8 or less, or in one embodiment, 3.7 or less.

In the present embodiment, in the chromatogram obtained by high performance liquid chromatography under the above conditions, the area ratio (decaprenylphenol/RQ9) of the peak containing decaprenylphenol to the peak containing RQ9 is not particularly limited, but may be within the range of 0.1 to 10.0, 0.1 to 8.0, 0.1 to 6.0, 0.1 to 5.0, 0.1 to 4.0, 0.5 to 4.0, or 1.0 to 4.0.

In the present embodiment, in the chromatogram obtained by high performance liquid chromatography under the above conditions, the area ratio (decaprenylphenol/RQ9) of the peak containing decaprenylphenol to the peak containing RQ9 is not particularly limited, but may be, for example, within the range of 0.1 to 10.0, in one embodiment, 0.1 to 9.0, in one embodiment, 0.1 to 8.0, in one embodiment, 0.1 to 7.0, in one embodiment, 0.1 to 6.0, in one embodiment, 0.1 to 5.0, in one embodiment, 0.1 to 4.0, in one embodiment, 0.2 to 4.0, in one embodiment, 0.3 to 4.0, in one embodiment, 0.4 to 4.0, in one embodiment, 0.5 to 4.0, in one embodiment, 0.6 to 4.0, in one embodiment, 0.7 to 4.0, in one embodiment, 0.8 to 4.0, in one embodiment, 0.9 to 4.0, in one embodiment, 1.0 to 4.0, in one embodiment, 1.2 to 4.0, in one embodiment, 1.4 to 4.0, in one embodiment, 1.6 to 4.0, in one embodiment, 1.8 to 4.0, in one embodiment, 2.0 to 4.0, in one embodiment, 2.2 to 4.0, in one embodiment, 2.3 to 4.0, in one embodiment, 2.4 to 4.0, in one embodiment, 2.6 to 4.0, in one embodiment, 2.8 to 4.0, in one embodiment, 3.0 to 4.0, in one embodiment, 3.2 to 4.0, in one embodiment, 3.4 to 4.0, in one embodiment, 3.6 to 4.0, in one embodiment, 3.6 to 3.8, or in one embodiment, 3.6 to 3.7.

In the present embodiment, in a chromatogram obtained by high performance liquid chromatography under the above conditions, the area ratio (RQ11/RQ9) of a peak containing RQ11 to a peak containing RQ9 is not particularly limited, but may be, for example, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, in one embodiment, 2.0 or more, in one embodiment, 2.2 or more, in one embodiment, 2.4 or more, in one embodiment, 2.6 or more, in one embodiment, 2.8 or more, in one embodiment, 3.0 or more, in one embodiment, 3.2 or more, in one embodiment, 3.3 or more, in one embodiment, 3.4 or more, in one embodiment, 3.6 or more, in one embodiment, 3.8 or more, in one embodiment, 4.0 or more, in one embodiment, 4.2 or more, in one embodiment, 4.4 or more, in one embodiment, 4.6 or more, in one embodiment, 4.8 or more, in one embodiment, 5.0 or more, in one embodiment, 5.5 or more, in one embodiment, 6.0 or more, in one embodiment, 6.5 or more, in one embodiment, 7.0 or more, in one embodiment, 7.5 or more, or in one embodiment 8.0 or more, and may be, for example, 15.0 or less, in one embodiment, 12.0 or less, in one embodiment, 10.0 or less, in one embodiment, 9.5 or less, in one embodiment, 9.0 or less, in one embodiment, 8.5 or less, in one embodiment, 8.4 or less, in one embodiment, 8.3 or less, in one embodiment, 8.2 or less, or in one embodiment, 8.1 or less.

In the present embodiment, in the chromatogram obtained by high performance liquid chromatography under the above conditions, the area ratio (RQ11/RQ9) of the peak containing RQ11 to the peak containing RQ9 is not particularly limited, but may be within the range of 1.0 to 15.0, 1.2 to 15.0, 1.4 to 12.0, 1.5 to 10.0, 1.8 to 9.5, or 2.0 to 9.0.

In the present embodiment, the area ratio (RQ11/RQ9) of the peak containing RQ11 to the peak containing RQ9 in the chromatogram obtained by high performance liquid chromatography under the above conditions is not particularly limited, but, for example, may be within the range of 1.0 to 15.0, in one embodiment, 1.0 to 12.0, in one embodiment, 1.0 to 10.0, in one embodiment, 1.0 to 9.5, in one embodiment, 1.0 to 9.0, in one embodiment, 1.0 to 8.5, in one embodiment, 1.0 to 8.4, in one embodiment, 1.0 to 8.3, in one embodiment, 1.0 to 8.2, in one embodiment, 1.0 to 8.1, in one embodiment, 1.1 to 8.1, in one embodiment, 1.2 to 8.1, in one embodiment, 1.3 to 8.1, in one embodiment, 1.4 to 8.1, in one embodiment, 1.5 to 8.1, in one embodiment, 1.6 to 8.1, in one embodiment, 1.7 to 8.1, in one embodiment, 1.8 to 8.1, in one embodiment, 1.9 to 8.1, in one embodiment, 2.0 to 8.1, in one embodiment, 2.2 to 8.1, in one embodiment, 2.4 to 8.1, in one embodiment, 2.6 to 8.1, in one embodiment, 2.8 to 8.1, in one embodiment, 3.0 to 8.1, in one embodiment, 3.2 to 8.1, in one embodiment, 3.3 to 8.1, in one embodiment, 3.4 to 8.1, in one embodiment, 3.6 to 8.1, in one embodiment, 3.8 to 8.1, in one embodiment, 4.0 to 8.1, in one embodiment, 4.2 to 8.1, in one embodiment, 4.4 to 8.1, in one embodiment, 4.6 to 8.1, in one embodiment, 4.8 to 8.1, in one embodiment, 5.0 to 8.1, in one embodiment, 5.5 to 8.1, in one embodiment, 6.0 to 8.1, in one embodiment, 6.5 to 8.1, in one embodiment, 7.0 to 8.1, in one embodiment, 7.5 to 8.1, in one embodiment, 8.0 to 8.1, in one embodiment, 1.2 to 15.0, in one embodiment, 1.4 to 12.0, in one embodiment, 1.5 to 10.0, in one embodiment, 1.8 to 9.5, or in one embodiment 2.0 to 9.0.

In the present embodiment, in the chromatogram obtained by high performance liquid chromatography under the above conditions, the area ratio (decaprenylphenol/RD) of the peak containing decaprenylphenol to the peak containing RD is not particularly limited and may be, for example, 0.1 or more, in one embodiment, 0.5 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, and may be, in one embodiment, 10 or less, in one embodiment, 5.0 or less, in one embodiment, 3.0 or less, in one embodiment, 2.5 or less, and in one embodiment, 2.0 or less.

In the present embodiment, in the chromatogram obtained by high performance liquid chromatography under the above conditions, the area ratio (decaprenylphenol/RD) of the peak containing decaprenylphenol to the peak containing RD is not particularly limited, but is within the range of preferably 0.1 to 10, more preferably 0.5 to 5.0, and more preferably 1.0 to 3.0.

In the present embodiment, in the chromatogram obtained by high performance liquid chromatography under the above conditions, the area ratio (decaprenylphenol/RD) of the peak containing decaprenylphenol to the peak containing RD is not particularly limited, but, for example, is within the range of 0.1 to 10, in one embodiment, 0.5 to 10, in one embodiment, 0.7 to 10, in one embodiment, 0.7 to 5.0, in one embodiment, 0.7 to 3.0, in one embodiment, 0.7 to 2.5, in one embodiment, 0.7 to 2.0, in one embodiment, 0.8 to 2.0, in one embodiment, 0.9 to 2.0, in one embodiment, 1.0 to 2.0, in one embodiment, 1.1 to 2.0, in one embodiment, 1.2 to 2.0, in one embodiment, 1.3 to 2.0, in one embodiment, 1.4 to 2.0, in one embodiment, 1.5 to 2.0, in one embodiment, 1.6 to 2.0, in one embodiment, 1.7 to 2.0, in one embodiment, 1.8 to 2.0, in one embodiment, 0.5 to 5.0, and in one embodiment, 1.0 to 3.0.

In the present embodiment, in the chromatogram obtained by high performance liquid chromatography under the above conditions, the area ratio (RD/RQ11) of the peak containing RD to the peak containing RQ11 is not particularly limited, but, for example, it is 0.1 or more, in one embodiment, 0.2 or more, specifically, 0.19 or more, in one embodiment, 0.20 or more, in one embodiment, 0.21 or more, and in one embodiment, 1.0 or less, in one embodiment, 0.8 or less, in one embodiment, 0.5 or less, specifically, 0.50 or less, in one embodiment, 0.49 or less, in one embodiment, 0.48 or less, and in one embodiment, 0.47 or less.

In the present embodiment, in the chromatogram obtained by high performance liquid chromatography under the above conditions, the area ratio (RD/RQ11) of the peak containing RD to the peak containing RQ11 is not particularly limited, but is within the range of preferably 0.1 to 1.0, more preferably 0.2 to 0.8, and more preferably 0.2 to 0.5.

In the present embodiment, in the chromatogram obtained by high performance liquid chromatography under the above conditions, the area ratio (RD/RQ11) of the peak containing RD to the peak containing RQ11 is not particularly limited, but is, for example, within the range of 0.1 to 1.0, in one embodiment, 0.2 to 0.8, in one embodiment, 0.2 to 0.5, specifically, 0.19 to 0.50, in one embodiment, 0.20 to 0.49, in one embodiment, 0.21 to 0.48, and in one embodiment, 0.21 to 0.47.

In the present embodiment, in the chromatogram obtained by high performance liquid chromatography under the above conditions, the area ratio (decaprenylphenol/RQ11) of the peak containing decaprenylphenol to the peak containing RQ11 is not particularly limited, but may be, for example, 0.01 or more, in one embodiment, 0.03 or more, in one embodiment, 0.05 or more, in one embodiment, 0.07 or more, in one embodiment, 0.08 or more, in one embodiment, 0.1 or more, in one embodiment, 0.2 or more, in one embodiment, 0.25 or more, in one embodiment, 0.3 or more, in one embodiment, 0.35 or more, in one embodiment, 0.4 or more, or in one embodiment, 0.45 or more, and may be, for example, 1.5 or less, in one embodiment, 1.1 or less, in one embodiment, 1.0 or less, in one embodiment 0.8 or less, in one embodiment, 0.7 or less, in one embodiment, 0.6 or less, or in one embodiment 0.5 or less.

In the present embodiment, in the chromatogram obtained by high performance liquid chromatography under the above conditions, the area ratio (decaprenylphenol/RQ11) of the peak containing decaprenylphenol to the peak containing RQ11 is not particularly limited, but may be within the range of 0.01 to 1.5, 0.03 to 1.0, 0.05 to 0.8, 0.07 to 0.7, 0.08 to 0.6, or 0.1 to 0.5.

In the present embodiment, in the chromatogram obtained by high performance liquid chromatography under the above conditions, the area ratio (decaprenylphenol/RQ11) of the peak containing decaprenylphenol to the peak containing RQ11 is not particularly limited, but may be, for example, within the range of 0.01 to 1.5, in one embodiment, 0.03 to 1.5, in one embodiment, 0.05 to 1.5, in one embodiment, 0.07 to 1.5, in one embodiment, 0.08 to 1.5, in one embodiment, 0.1 to 1.5, in one embodiment, 0.2 to 1.5, in one embodiment, 0.2 to 1.1, in one embodiment, 0.2 to 1.0, in one embodiment, 0.2 to 0.8, in one embodiment, 0.2 to 0.7, in one embodiment, 0.2 to 0.6, in one embodiment, 0.2 to 0.5, in one embodiment, 0.25 to 0.5, in one embodiment, 0.3 to 0.5, in one embodiment, 0.35 to 0.5, in one embodiment, 0.4 to 0.5, in one embodiment, 0.45 to 0.5, in one embodiment, 0.03 to 1.0, in one embodiment, 0.05 to 0.8, in one embodiment, 0.07 to 0.7, in one embodiment, 0.08 to 0.6, or in one embodiment 0.1 to 0.5.

In the present embodiment, in the chromatogram obtained by high performance liquid chromatography under the above conditions, the ratio ((decaprenylphenol+RQ9)/QH) of the total area of the peak containing decaprenylphenol and the peak containing reduced coenzyme Q9 (RQ9) to the area of the peak containing reduced coenzyme Q10 (QH) is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.025 or more, in one embodiment, 0.030 or more, in one embodiment, 0.10 or less, in one embodiment, 0.050 or less, in one embodiment, 0.040 or less, in one embodiment, 0.030 or less, in one embodiment, 0.020 or less, in one embodiment, 0.025 or less, in one embodiment, 0.020 or less, and in one embodiment, 0.015 or less.

In the present embodiment, in the chromatogram obtained by high performance liquid chromatography under the above conditions, the ratio ((decaprenylphenol+RQ9)/QH) of the total area of the peak containing decaprenylphenol and the peak containing reduced coenzyme Q9 (RQ9) to the area of the peak containing reduced coenzyme Q10 (QH) is not particularly limited, but is, for example, within the range of 0.001 to 0.10, in one embodiment, 0.005 to 0.050, in one embodiment, 0.007 to 0.040, in one embodiment, 0.008 to 0.030, in one embodiment, 0.009 to 0.025, in one embodiment, 0.010 to 0.020, and in one embodiment 0.010 to 0.015.

In the present embodiment, in the chromatogram obtained by high performance liquid chromatography under the above conditions, the ratio ((decaprenylphenol+RQ11)/QH) of the total area of the peak containing decaprenylphenol and the peak containing reduced coenzyme Q11 (RQ11) to the area of the peak containing reduced coenzyme Q10 (QH) is not particularly limited, but, for example, may be 0.001 or more, in one embodiment, 0.005 or more, in one embodiment, 0.006 or more, in one embodiment, 0.007 or more, in one embodiment, 0.008 or more, in one embodiment, 0.009 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.025 or more, in one embodiment, 0.030 or more, in one embodiment, 0.10 or less, in one embodiment, 0.050 or less, in one embodiment, 0.040 or less, in one embodiment, 0.035 or less, in one embodiment, 0.032 or less, and in one embodiment, 0.031 or less.

In the present embodiment, in the chromatogram obtained by high performance liquid chromatography under the above conditions, the ratio ((decaprenylphenol+RQ11)/QH) of the total area of the peak containing decaprenylphenol and the peak containing reduced coenzyme Q11 (RQ11) to the peak containing reduced coenzyme Q10 (QH) is not particularly limited but is, for example, within the range of 0.001 to 0.10, in one embodiment, 0.005 to 0.050, in one embodiment, 0.007 to 0.040, in one embodiment, 0.008 to 0.035, in one embodiment, 0.009 to 0.032, in one embodiment, 0.010 to 0.031, in one embodiment, 0.012 to 0.031, in one embodiment, 0.013 to 0.031, in one embodiment, 0.014 to 0.031, in one embodiment, 0.015 to 0.031, in one embodiment, 0.020 to 0.031, and in one embodiment, 0.025 to 0.031.

In the present embodiment, in the chromatogram obtained by high performance liquid chromatography under the above conditions, the ratio ((RQ9+RQ11)/QH) of the total area of the peaks containing reduced coenzyme Q9 (RQ9) and the peaks containing reduced coenzyme Q11 (RQ11) to the area of the peak containing reduced coenzyme Q10 (QH) is not particularly limited, but, for example, may be 0.001 or more, in one embodiment, 0.005 or more, in one embodiment, 0.010 or more, in one embodiment, 0.011 or more, in one embodiment, 0.012 or more, in one embodiment, 0.013 or more, in one embodiment, 0.014 or more, in one embodiment, 0.015 or more, in one embodiment, 0.016 or more, in one embodiment, 0.017 or more, in one embodiment, 0.018 or more, in one embodiment, 0.019 or more, in one embodiment, 0.020 or more, in one embodiment, 0.021 or more, in one embodiment, 0.022 or more, and may be, in one embodiment, 0.10 or less, in one embodiment, 0.050 or less, in one embodiment, 0.040 or less, in one embodiment, 0.035 or less, in one embodiment, 0.030 or less, in one embodiment, 0.029 or less, in one embodiment, 0.028 or less, in one embodiment, 0.027 or less, in one embodiment, 0.026 or less, and in one embodiment 0.025 or less.

In the present embodiment, in the chromatogram obtained by high performance liquid chromatography under the above conditions, the ratio ((RQ9+RQ11)/QH) of the total area of the peak containing reduced coenzyme Q9 (RQ9) and the peak containing reduced coenzyme Q11 (RQ11) to the peak containing reduced coenzyme Q10 (QH) is not particularly limited, but is, for example, within the range of 0.001 to 0.10, in one embodiment, 0.005 to 0.050, in one embodiment, 0.010 to 0.040, in one embodiment, 0.011 to 0.035, in one embodiment, 0.012 to 0.030, in one embodiment, 0.013 to 0.029, in one embodiment, 0.014 to 0.028, in one embodiment, 0.014 to 0.027, in one embodiment, 0.015 to 0.026, in one embodiment, 0.016 to 0.026, in one embodiment, 0.017 to 0.026, in one embodiment, 0.018 to 0.026, in one embodiment, 0.020 to 0.026, and in one embodiment, 0.022 to 0.026.

In the present embodiment, in the present embodiment, in a chromatogram obtained by high performance liquid chromatography under the above conditions, the area ratio (total value of the peak areas of the QH analogs/peak area of QH) of peaks containing QH analogs (RD, RQ11, RQ9, and decaprenylphenol) to a peak containing QH is not particularly limited, but, for example, may be 0.001 or more, in one embodiment, 0.006 or more, in one embodiment, 0.007 or more, in one embodiment, 0.012 or more, in one embodiment, 0.02 or more, in one embodiment, 0.022 or more, in one embodiment, 0.024 or more, in one embodiment, 0.03 or more, in one embodiment, 0.033 or more, in one embodiment, 0.035 or more, in one embodiment, 0.038 or more, in one embodiment, 0.10 or less, in one embodiment, 0.08 or less, in one embodiment, 0.06 or less, in one embodiment, 0.05 or less, and in one embodiment 0.04 or less.

In the present embodiment, in the present embodiment, in the chromatogram obtained by high performance liquid chromatography under the above conditions, the area ratio (total value of the peak areas of QH analogs/peak area of QH) of the peaks containing QH analogs (RD, RQ11, RQ9, and decaprenylphenol) to the peak containing QH is not particularly limited, but is within the range of preferably 0.001 to 0.10, more preferably 0.006 to 0.08, more preferably 0.007 to 0.06, more preferably 0.012 to 0.05, and more preferably 0.02 to 0.04.

In the present embodiment, in the chromatogram obtained by high performance liquid chromatography under the above conditions, the area ratio (total value of the peak areas of QH analogs/peak area of QH) of the peaks containing QH analogs (RD, RQ11, RQ9, and decaprenylphenol) to the peak containing QH is not particularly limited, but is, for example, within the range of 0.001 to 0.10, in one embodiment, 0.001 to 0.08, in one embodiment, 0.001 to 0.06, in one embodiment, 0.001 to 0.05, in one embodiment, 0.001 to 0.04, in one embodiment, 0.006 to 0.04, in one embodiment, 0.007 to 0.04, in one embodiment, 0.012 to 0.04, in one embodiment, 0.02 to 0.04, in one embodiment, 0.022 to 0.04, in one embodiment, 0.024 to 0.04, in one embodiment, 0.03 to 0.04, in one embodiment, 0.033 to 0.04, in one embodiment, 0.035 to 0.04, in one embodiment, 0.038 to 0.04, in one embodiment, 0.006 to 0.08, in one embodiment, 0.007 to 0.06, and in one embodiment 0.012 to 0.05.

The substances other than reduced coenzyme Q10 and its analogs, reduced demethoxy Q10, and reduced coenzyme Q9 contained in the coexisting state or the oral composition of one or more embodiments of the present invention are not particularly limited, and examples thereof include an excipient, a disintegrant, a lubricant, a binder, an emulsifier, a colorant, an anti-caking agent, an absorption enhancer, a dissolution aid, a stabilizer, a flavoring agent, fats and oils, a surfactant, a higher fatty acid, ethanol, water, an active ingredient other than reduced coenzyme Q10, an antioxidant, and the like.

Further, the form of the coexisting state according to one or more embodiments of the present invention is not particularly limited, and can be appropriately selected as necessary. Specifically, as the form of the coexisting state, examples thereof include oral preparations such as tablets, powders, chewable tablets, pills, capsules, granules, fine granules, sustained-release preparations, suspensions, emulsion preparations, syrups, elixirs, and the like, and non-oral preparations such as injections, suppositories, topical preparations, patch preparations, and the like. Since reduced coenzyme Q10 has excellent oral absorbability, as the form of the coexisting state, the form of an oral preparation is particularly preferred. Furthermore, the form of the coexisting state according to one or more embodiments of the present invention can also be in the form of a capsule, for example, a capsule obtained by dissolution of reduced coenzyme Q10 in an oil component and sealing it in a capsule. Among these, the form of the coexisting state is preferably a composition in solid form.

Further, the form of the oral composition according to one or more embodiments of the present invention is not particularly limited, and can be appropriately selected as necessary. Specifically, examples thereof include oral preparations such as tablets, powders, chewable tablets, pills, capsules, granules, fine granules, sustained-release preparations, suspensions, emulsion preparations, syrups, elixirs, and the like. Furthermore, the oral composition according to one or more embodiments of the present invention can also be in the form of a capsule, for example, a capsule obtained by dissolution of reduced coenzyme Q10 in an oil component and sealing it in a capsule. Among these, an oral composition in solid form is preferred.

Hereinafter, the case where it is used as a composition in solid form, such as granules or tablets, will be described.

The form of a solid state in the coexisting state or the oral composition is not particularly limited, and examples thereof include forms such as powder, granules, tablet, hard capsule, and the like.

An emulsifier may be added to the coexisting state or the oral composition. The type of emulsifier to be added is not particularly limited, but the use of an emulsifier that is acceptable for food, cosmetic, and/or pharmaceutical use is particularly preferred. For example, the use of an emulsifier having an HLB of 1 or more and 17 or less, preferably 2 or more and 16 or less, is possible. The use of two or more emulsifiers in combination is allowed. The preferably used specific examples of emulsifiers include one or two or more emulsifiers selected from the group consisting of glycerin fatty acid ester, sucrose fatty acid ester, retinol fatty acid ester, propylene glycol fatty acid ester, sorbitan fatty acid ester, and polysorbate.

From the viewpoint of suppressing the oxidation of reduced coenzyme Q10, among the above emulsifiers, emulsifiers that are neither in powder nor flake form are preferably used. Examples of the emulsifiers that are neither in powder nor flake form include emulsifiers that are in a liquid-to-sol form, a gel form, or a soft solid, and emulsifiers that are liquid, viscid liquid, viscous liquid, paste, pellets, waxy mass, wax, soft solid, or semi-solid are more preferred. As the emulsifier, an emulsifier having the above physical properties at 50° C. is preferred, and an emulsifier having the above physical properties at 25° C. is most preferred.

Examples of the emulsifiers that are neither in powder nor in flake form include emulsifiers in which the melting onset point in a measurement by a differential scanning calorimeter (DSC) with the heating rate set to 1° C./min or more and 20° C./min or less is 50° C. or lower, preferably 40° C. or lower, more preferably 25° C. or lower.

From another viewpoint, examples of the emulsifiers include emulsifiers in which the viscosity, in a measurement with the use of a B-type viscometer at a rotational speed of 10 rpm and a sample temperature of 50° C., is, for example, 150,000 mPa·s or less, preferably 100,000 mPa·s or less, more preferably 50,000 mPa·s or less, more preferably 30,000 mPa·s or less, and more preferably 25,000 mPa·s or less. The lower limit of the viscosity is not particularly limited as long as the viscosity is greater than 0 mPa·s, the viscosity is more preferably 1 mPa·s or more, more preferably 5 mPa·s or more, and more preferably 10 mPa·s or more. That is, the above viscosity can be, for example, within the range of 1 to 150,000 mPa·s, preferably within the range of 5 to 30,000 mPa·s, and more preferably within the range of 10 to 25,000 mPa·s.

The specific examples of the emulsifier include one or more selected from an ester compound of a polyol selected from monoglycerin, polyglycerin, sorbitan, polyoxyethylene sorbitan, sucrose, propylene glycol, polypropylene glycol, ethylene glycol, and polyethylene glycol, and a fatty acid which may have a substituent, and lecithin.

In polyglycerin, the number of units of glycerin may be 2 or more, and is preferably 2 or more and 10 or less. For example, diglycerin, triglycerin, tetraglycerin, pentaglycerin, hexaglycerin, and decaglycerin can be exemplified.

In polyoxyethylene sorbitan, the number of units of oxyethylene may be 2 or more, preferably 10 or more and 30 or less, more preferably 15 or more and 25 or less.

In polypropylene glycol, the number of units of propylene glycol may be 2 or more, and is preferably 2 or more and 10 or less.

In polyethylene glycol, the number of units of ethylene glycol may be 2 or more, and preferably is 2 or more and 10 or less.

Examples of the fatty acids that may have a substituent include straight-chain or branched-chain monocarboxylic or dicarboxylic fatty acids having 4 or more and 24 or less carbon atoms. As substituents, a hydroxy group and an acetoxy group can be exemplified. It is preferred that the number of substituents is 2 or less. Examples of the fatty acids that may have a substituent include lauric acid, oleic acid, caprylic acid, stearic acid, behenic acid, ricinoleic acid, succinic acid, and diacetyl tartaric acid.

In the ester compound of the polyol and the fatty acid, the number of bonds of the fatty acid to one molecule of the polyol is not particularly limited, and can be appropriately adjusted according to the HLB of the emulsifier for the intended purpose.

From the viewpoint of suppressing oxidation of reduced coenzyme Q10, the number of bonds of the fatty acid to one molecule of the polyol can be, for example, 12 or less, preferably 10 or less, more preferably 7 or less, more preferably 6 or less, more preferably 5 or less, more preferably 3 or less, more preferably 2 or less, and more preferably, is 1.

Specific examples of the ester compound of the polyol and the fatty acid include diglyceryl monooleate, monoglyceryl monocaprylate, diglyceryl monocaprylate, decaglyceryl pentaoleate, tetraglyceryl pentaoleate, pentaglyceryl trioleate, decaglyceryl monolaurate, hexaglyceryl monocaprylate, hexaglyceryl monooleate, pentaglyceryl monostearate, tetraglyceryl tristearate, decaglyceryl monobehenate, monostearate of mono- and diglycerides, monoglyceryl monooleate, glyceryl monostearate succinate, monoglyceryl succinate, glyceryl monostearate diacetyl tartrate, propylene glycol monooleate, sorbitan monooleate, sorbitan monostearate, sorbitan tristearate, monoglyceryl monolaurate, diglyceryl monolaurate, diglyceryl monomyristate, tetraglyceryl pentastearate, polyoxyethylene sorbitan monooleate, polyoxyethylene sorbitan monolaurate, polyoxyethylene sorbitan monostearate, condensed ricinoleic acid pentaglyceride, sucrose stearate, sucrose erucate, and sucrose oleate.

From the viewpoint of suppressing oxidation of reduced coenzyme Q10, the fatty acid in the ester compound of the polyol and the fatty acid is more preferably an unsaturated fatty acid. Examples of the unsaturated fatty acid include, for example, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, alpha-linolenic acid, gamma-linolenic acid, pinolenic acid, alpha-eleostearic acid, beta-eleostearic acid, Mead acid, dihomo-gamma-linolenic acid, eicosatrienoic acid, stearidonic acid, arachidonic acid, eicosatetraenoic acid, adrenic acid, boseopentaenoic acid, eicosapentaenoic acid, Osbond acid, clupanodonic acid, tetracosapentaenoic acid, docosahexaenoic acid, cetoleic acid, ricinoleic acid, and condensed ricinoleic acid, and oleic acid, condensed ricinoleic acid, linoleic acid, and erucic acid are more preferred, and oleic acid is particularly preferred.

Specific examples of the ester compounds of the polyol and the unsaturated fatty acid include monoglyceryl monooleate, monoglyceryl mono- and dioleate, monoglyceryl dioleate, mono- and diglyceryl monooleate, diglyceryl monooleate, diglyceryl mono- and dioleate, diglyceryl dioleate, diglyceryl trioleate, triglyceryl monooleate, triglyceryl dioleate, triglyceryl trioleate, triglyceryl tetraoleate, tetraglyceryl monooleate, tetraglyceryl dioleate, tetraglyceryl trioleate, tetraglyceryl tetraoleate, tetraglyceryl pentaoleate, pentaglyceryl monooleate, pentaglyceryl dioleate, pentaglyceryl trioleate, pentaglyceryl tetraoleate, pentaglyceryl pentaoleate, pentaglyceryl hexaoleate, hexaglyceryl monooleate, hexaglyceryl dioleate, hexaglyceryl trioleate, hexaglyceryl tetraoleate, hexaglyceryl pentaoleate, hexaglyceryl hexaoleate, hexaglyceryl heptaoleate, decaglyceryl monooleate, decaglyceryl dioleate, decaglyceryl trioleate, decaglyceryl tetraoleate, decaglyceryl pentaoleate, decaglyceryl hexaoleate, decaglyceryl heptaoleate, decaglyceryl octaoleate, decaglyceryl nonaoleate, decaglyceryl decaoleate, decaglyceryl dodecaoleate, diacetylglycerin monooleate, glyceryl monooleate lactate, glyceryl monooleate succinate, glyceryl monooleate citrate, glyceryl monooleate diacetyl tartrate, sucrose oleate, propylene glycol monooleate, sorbitan monooleate, sorbitan dioleate, sorbitan trioleate, polyoxyethylene sorbitan monooleate, phosphatidylcholine monopalmitate monooleate, phosphatidylcholine dilinoleate, monoglyceryl monoerucate, monoglyceryl mono- and dierucate, monoglyceryl dierucate, mono- and diglyceryl monoerucate, diglyceryl monoerucate, diglyceryl mono- and dierucate, diglyceryl dierucate, diglyceryl trierucate, triglyceryl monoerucate, triglyceryl dierucate, triglyceryl trierucate, triglyceryl tetraerucate, tetraglyceryl monoerucate, tetraglyceryl dierucate, tetraglyceryl trierucate, tetraglyceryl tetraerucate, tetraglyceryl pentaerucate, pentaglyceryl monoerucate, pentaglyceryl dierucate, pentaglyceryl trierucate, pentaglyceryl tetraerucate, pentaglyceryl pentaerucate, pentaglyceryl hexaerucate, hexaglyceryl monoerucate, hexaglyceryl dierucate, hexaglyceryl trierucate, hexaglyceryl tetraerucate, hexaglyceryl pentaerucate, hexaglyceryl hexaerucate, hexaglyceryl heptaerucate, decaglyceryl monoerucate, decaglyceryl dierucate, decaglyceryl trierucate, decaglyceryl tetraerucate, decaglyceryl pentaerucate, decaglyceryl hexaerucate, decaglyceryl heptaerucate, decaglyceryl octaerucate, decaglyceryl nonaerucate, decaglyceryl decaerucate, decaglyceryl dodecaerucate, diacetylglycerin monoerucate, glyceryl monoerucate lactate, glyceryl monoerucate succinate, glyceryl monoerucate citrate, glyceryl monoerucate diacetyl tartrate, sucrose erucate, propylene glycol monoerucate, sorbitan monoerucate, sorbitan dierucate, sorbitan trierucate, condensed ricinoleic acid monoglyceride, condensed ricinoleic acid diglyceride, condensed ricinoleic acid triglyceride, condensed ricinoleic acid tetraglyceride, condensed ricinoleic acid pentaglyceride, condensed ricinoleic acid hexaglyceride, condensed ricinoleic acid heptaglyceride, and condensed ricinoleic acid decaglyceride.

From the viewpoint of suppression of oxidation of reduced coenzyme Q10, as the ester compound of the polyol and the fatty acid, a polyoxyethylene sorbitan fatty acid ester is also preferably used. Specific examples of the polyoxyethylene sorbitan fatty acid ester include polyoxyethylene sorbitan monooleate, polyoxyethylene sorbitan monolaurate, and polyoxyethylene sorbitan monostearate.

In the method for improving an in vivo absorbability of reduced coenzyme Q10 or an oral composition of the reduced coenzyme Q10 according to one or more embodiments of the present invention, in order to ensure oxidative stability, particularly in a solid dosage form, preferably an antioxidant is caused to coexist or is contained. Here, the type of the antioxidant is not particularly limited. The use of two or more kinds of antioxidants in combination is permitted. Specific examples of antioxidants preferably include those that are solid at room temperature, and examples thereof include one or more antioxidants selected from the group consisting of ascorbic acid, ascorbate, erythorbic acid, and erythorbate.

The counterion of each of ascorbate and erythorbate is not limited, but can be one or more metal salts, each independently selected from the group consisting of sodium salts, potassium salts, calcium salts, and magnesium salts.

As an antioxidant, the use of an antioxidant acceptable in foods or pharmaceuticals is particularly preferred. As the antioxidant, an ascorbate is preferred, and it is particularly preferred that the ascorbate be one or more selected from sodium ascorbate and calcium ascorbate.

In the method for improving an in vivo absorbability of reduced coenzyme Q10 or an oral composition of the reduced coenzyme Q10 according to one or more embodiments of the present invention, preferably a binder is caused to coexist or is contained. The binder can be utilized to bind respective components such as reduced coenzyme Q10, an emulsifier, an antioxidant, and the like, and to form a composition in a solid form.

The type of binder is not limited. Use may be made of two or more binders in combination. Specific examples of binders include one or more selected from celluloses and starches.

Examples of the celluloses include hydroxypropyl cellulose, hydroxypropyl methylcellulose, hydroxymethyl cellulose, carboxymethyl cellulose, crystalline cellulose, cellulose powder, methyl cellulose, ethyl cellulose, and salts thereof and the like. A particularly preferred binder is one or more selected from the group consisting of hydroxypropyl cellulose, hydroxypropyl methylcellulose, and sodium carboxymethyl cellulose.

Examples of the starches include wheat starch, potato starch, sweet potato starch, corn starch, dextrin, hydroxypropyl starch, acetate starch, oxidized starch, partially alpha-converted starch, and the like.

As a binder, the use of a binder that is acceptable as a food or a pharmaceutical is particularly preferred.

In the method for improving an in vivo absorbability of reduced coenzyme Q10 or an oral composition of the reduced coenzyme Q10 according to one or more embodiments of the present invention, the blending proportion of each component can be appropriately set according to the dosage form and the like.

In the method for improving an in vivo absorbability of reduced coenzyme Q10 or an oral composition of the reduced coenzyme Q10 according to one or more embodiments of the present invention, in a case where an antioxidant is caused to coexist or is contained, the content of an antioxidant is, with respect to 100 parts by weight of reduced coenzyme Q10, for example, 1 part by weight or more and 9900 parts by weight or less. The lower limit of the content of the antioxidant is, with respect to 100 parts by weight of reduced coenzyme Q10, preferably 20 parts by weight or more, more preferably 50 parts by weight or more, more preferably 55 parts by weight or more. The upper limit of the content of an antioxidant is, with respect to 100 parts by weight of reduced coenzyme Q10, preferably 5000 parts by weight or less, more preferably 1000 parts by weight or less, more preferably 500 parts by weight or less, more preferably 200 parts by weight or less, and more preferably 120 parts by weight or less. That is, the content of the antioxidant with respect to 100 parts by weight of reduced coenzyme Q10 is within the range of preferably 20 to 5000 parts by weight, more preferably 50 to 1000 parts by weight, and more preferably 55 to 500 parts by weight.

In the method for improving an in vivo absorbability of reduced coenzyme Q10 or an oral composition of the reduced coenzyme Q10 according to one or more embodiments of the present invention, in the case where an emulsifier is caused to coexist or is contained, the content of an emulsifier is, with respect to 100 parts by weight of reduced coenzyme Q10, for example, from 1 part by weight or more to 9900 parts by weight or less. The lower limit of the content of the emulsifier is, with respect to 100 parts by weight of reduced coenzyme Q10, preferably 3 parts by weight or more, more preferably 5 parts by weight or more, and more preferably 8 parts by weight or more. The upper limit of the content of the emulsifier is, with respect to 100 parts by weight of reduced coenzyme Q10, preferably 5000 parts by weight or less, more preferably 1000 parts by weight or less, more preferably 500 parts by weight or less, more preferably 200 parts by weight or less, more preferably 150 parts by weight or less, more preferably 100 parts by weight or less, more preferably 50 parts by weight or less, more preferably 30 parts by weight or less. That is, the content of an emulsifier with respect to 100 parts by weight of reduced coenzyme Q10 can be within the range of preferably 3 to 5000 parts by weight, more preferably 5 to 1000 parts by weight, and more preferably 8 to 500 parts by weight.

In the method for improving an in vivo absorbability of reduced coenzyme Q10 or an oral composition of the reduced coenzyme Q10 according to one or more embodiments of the present invention, in the case where a binder is caused to coexist or is contained, the lower limit of the content of the binder is, with respect to 100 parts by weight of reduced coenzyme Q10, for example 1 parts by weight or more, preferably 3 parts by weight or more, more preferably 5 parts by weight or more, more preferably 8 parts by weight or more, and more preferably 10 parts by weight or more. Furthermore, the upper limit of the content of the binder is, with respect to 100 parts by weight of reduced coenzyme Q10, for example 600 parts by weight or less, preferably 550 parts by weight or less, more preferably 500 parts by weight or less, more preferably 450 parts by weight or less, more preferably 400 parts by weight or less. That is, the content of the binder with respect to 100 parts by weight of reduced coenzyme Q10 can be within the range of preferably 1 to 600 parts by weight, more preferably 3 to 550 parts by weight, and more preferably 5 to 500 parts by weight.

In the method for improving an in vivo absorbability of reduced coenzyme Q10 or an oral composition of the reduced coenzyme Q10 according to one or more embodiments of the present invention, in a case where an antioxidant is caused to coexist or is contained, the lower limit of the content of the antioxidant is, with respect to total weight of total components of coexisting state or total weight of oral composition, for example, 1 wt % or more, preferably 2 wt % or more, more preferably 4 wt % or more, more preferably 5 wt % or more, and the upper limit of the content of the antioxidant is, for example, 99 wt % or less, preferably 90 wt % or less, more preferably 80 wt % or less, more preferably 70 wt % or less, more preferably 60 wt % or less. That is, the content of the antioxidant can be, for example, within the range of 1 to 99 wt %, preferably within the range of 2 to 90 wt %, more preferably within the range of 4 to 80 wt %, and more preferably within the range of 5 to 60 wt %.

In the method for improving an in vivo absorbability of reduced coenzyme Q10 or an oral composition of the reduced coenzyme Q10 according to one or more embodiments of the present invention, in a case where an emulsifier is caused to coexist or is contained, the lower limit of the content of the emulsifier is, with respect to total weight of total components of coexisting state or total weight of oral composition, for example, 0.5 wt % or more, preferably 0.7 wt % or more, more preferably 0.9 wt % or more, and the upper limit of the content of the emulsifier is, for example, 99 wt % or less, preferably 50 wt % or less, more preferably 30 wt % or less, more preferably 25 wt % or less, more preferably 20 wt % or less, more preferably 15 wt % or less, more preferably 10 wt % or less. Namely, the content of the emulsifier can be, for example, within the range of 0.5 to 99 wt %, preferably 0.7 to 50 wt %, more preferably 0.9 to 10 wt %.

In the method for improving an in vivo absorbability of reduced coenzyme Q10 or an oral composition of the reduced coenzyme Q10 according to one or more embodiments of the present invention, in a case where a binder is caused to coexist or is contained, the lower limit of the content of the binder is, with respect to total weight of total components of coexisting state or total weight of oral composition, for example, 1 wt % or more, preferably 10 wt % or more, more preferably 20 wt % or more, and the upper limit of the content of the binder is, for example, 80 wt % or less, preferably 70 wt % or less, more preferably 60 wt % or less. That is, the content of the binder can be, for example, within the range of 1 to 80 wt %, preferably 10 to 70 wt %, more preferably 20 to 60 wt %.

Next, a description will be given of the coexisting method in the method for improving an in vivo absorbability, or the production method for the oral composition, with respect to reduced coenzyme Q10 of one or more embodiments of the present invention. In one or more embodiments of the present invention, the coexisting state of reduced coenzyme Q10 with reduced demethoxy Q10 and reduced coenzyme Q9 can be formed in a composition in a state of liquid or in a state of suspension by combining reduced coenzyme Q10, reduced demethoxy Q10, and reduced coenzyme Q9 in a solvent. In one or more embodiments of the present invention, the coexisting state of reduced coenzyme Q10 with reduced demethoxy Q10 and reduced coenzyme Q9 can be formed in a composition in a state of suspension or in a state of solid by, from a solution containing reduced coenzyme Q10, reduced demethoxy Q10, and reduced coenzyme Q9, precipitation and optional separation of a composition in solid form that contains those reduced coenzyme Q10, reduced demethoxy Q10, and reduced coenzyme Q9. In one or more embodiments of the present invention, an oral composition of reduced coenzyme Q10 can be produced by combining reduced coenzyme Q10, reduced demethoxy Q10, and reduced coenzyme Q9 in a solvent. In one or more embodiments of the present invention, the oral composition of reduced coenzyme Q10 can be produced by, from a solution containing reduced coenzyme Q10, reduced demethoxy Q10, and reduced coenzyme Q9, precipitation and optional separation of a composition in solid form that contains those reduced coenzyme Q10, reduced demethoxy Q10, and reduced coenzyme Q9. In a case where a method of one or more embodiments of the present invention or production of the oral composition is carried out by precipitation of the composition in solid form, at the time of precipitating and separating the composition in solid form, by controlling the blending ratio of each component in the solution and/or the conditions of the separation step so that the peak area ratio in the chromatogram for RD and RQ9 in the composition is within a specific range, it is possible to obtain the coexisting state or the oral composition in which the in vivo absorbability, particularly the digestive fluid solubility, of reduced coenzyme Q10 (QH) has been improved. Specifically, in the chromatogram of the coexisting state or the oral composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be controlled to be 0.3 or more. The separation step may be carried out so that the above peak area ratio (RD/RQ9) may be 0.5 or more, 0.8 or more, 1.0 or more, 1.5 or more, or 2.0 or more. The upper limit of the above peak area (RD/RQ9) is not particularly limited, but, the separation step may be performed so that the above peak area ratio (RD/RQ9) may be 10.0 or less, 8.0 or less, 6.0 or less, 4.0 or less, or 3.0 or less. That is, the above peak area ratio may be within the range of 0.3 to 10.0, 0.5 to 10.0, 0.5 to 3.0, 0.8 to 8.0, 1.0 to 6.0, 1.0 to 2.0, 1.5 to 4.0, or 2.0 to 3.0.

In another embodiment, specifically, in the chromatogram of the composition obtained by high performance liquid chromatography under the following conditions:
a column: a C18 column (150 mm in length and 4.6 mm in inner diameter),
a mobile phase composition: CH₃OH:C₆H₁₄=9:1 (v/v),
a mobile phase flow rate: 1 mL/min, and
a detection wavelength: 190 nm,
   an area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 may be controlled to be 0.3 or more. The above peak area ratio (RD/RQ9) may be, for example, 0.4 or more, in one embodiment, 0.5 or more, in one embodiment, 0.6 or more, in one embodiment, 0.7 or more, in one embodiment, 0.8 or more, in one embodiment, 0.9 or more, in one embodiment, 1.0 or more, in one embodiment, 1.1 or more, in one embodiment, 1.2 or more, in one embodiment, 1.3 or more, in one embodiment, 1.4 or more, in one embodiment, 1.5 or more, in one embodiment, 1.6 or more, in one embodiment, 1.7 or more, in one embodiment, 1.8 or more, in one embodiment, 1.9 or more, or in one embodiment, 2.0 or more. The upper limit of the above peak area ratio (RD/RQ9) is not particularly limited, but may be, for example, 10.0 or less, in one embodiment, 9.0 or less, in one embodiment, 8.0 or less, in one embodiment, 7.0 or less, in one embodiment, 6.0 or less, in one embodiment, 5.0 or less, in one embodiment, 4.0 or less, in one embodiment, 3.0 or less, in one embodiment, 2.8 or less, in one embodiment, 2.6 or less, in one embodiment, 2.4 or less, in one embodiment, 2.2 or less, or in one embodiment, 2.1 or less. That is, the above peak area ratio may be 0.3 or more, and, for example, may be within the range of 0.3 to 10.0, in one embodiment, 0.3 to 9.0, in one embodiment, 0.3 to 8.0, in one embodiment, 0.3 to 7.0, in one embodiment, 0.3 to 6.0, in one embodiment, 0.3 to 5.0, in one embodiment, 0.3 to 4.0, in one embodiment, 0.3 to 3.0, in one embodiment, 0.3 to 2.8, in one embodiment, 0.3 to 2.6, in one embodiment, 0.3 to 2.4, in one embodiment, 0.3 to 2.2, in one embodiment, 0.3 to 2.1, in one embodiment, 0.4 to 2.1, in one embodiment, 0.5 to 2.1, in one embodiment, 0.6 to 2.1, in one embodiment, 0.7 to 2.1, in one embodiment, 0.8 to 2.1, in one embodiment, 0.9 to 2.1, in one embodiment, 1.0 to 2.1, in one embodiment, 1.1 to 2.1, in one embodiment, 1.2 to 2.1, in one embodiment, 1.3 to 2.1, in one embodiment, 1.4 to 2.1, in one embodiment, 1.5 to 2.1, in one embodiment, 1.6 to 2.1, in one embodiment, 1.7 to 2.1, in one embodiment, 1.8 to 2.1, in one embodiment, 1.9 to 2.1, in one embodiment, 2.0 to 2.1, in one embodiment, 0.5 to 10.0, in one embodiment, 0.5 to 3.0, in one embodiment, 0.8 to 8.0, in one embodiment, 1.0 to 6.0, in one embodiment, 1.0 to 2.0, in one embodiment, 1.5 to 4.0, or in one embodiment, 2.0 to 3.0.

In one or more embodiments of the present invention, the separation step may be performed by adding a seed crystal of reduced coenzyme Q10 to a solution containing reduced coenzyme Q10, reduced demethoxy Q10, and reduced coenzyme Q9, and cooling the solution. Thereby, it is possible to cause precipitation of the reduced coenzyme Q10 crystal and to obtain a composition in solid form.

A solution containing reduced coenzyme Q10, reduced demethoxy Q10, and reduced coenzyme Q9 may be a homogeneous solution state with dissolution of reduced coenzyme Q10 and the like in an organic solvent, or may be a slurry state with a part remaining without dissolution, however, a homogeneous solution state is preferred. In addition, the solution may contain various compounds in addition to the above reduced coenzyme Q10, reduced demethoxy Q10, and reduced coenzyme Q9, and examples include QH analogs such as reduced coenzyme Q11 and decaprenylphenol.

It is to be noted that, as for the reduced coenzyme Q10 for use in the above solution, there is no limitation as to whether it is in a crystal state or an amorphous state, and its crystal polymorphism is also not limited. Therefore, the use of conventionally known Form I type reduced coenzyme Q10 is also possible. Also, since it is possible to increase its purity in crystal precipitation, it may be one having analogs, or unpurified or crudely purified reduced coenzyme Q10. Furthermore, an extract solution of reduced coenzyme Q10 obtained by conventionally known methods, and/or a reaction solution that contains reduced coenzyme Q10 obtained from oxidized coenzyme Q10 by a known reduction method, as is, or, if necessary, after purification and/or solvent substitution, can be used as the solution in the separation step. For example, in a solution in which oxidized coenzyme Q10 and reduced coenzyme Q10 are present together, a solution obtained after subjecting only oxidized coenzyme Q10 to reduction treatment by a known reduction method, or, in a solution containing only oxidized coenzyme Q10, a solution obtained after subjecting oxidized coenzyme Q10 to reduction treatment by a known reduction method, or, in a solution in which oxidized coenzyme Q10 and components other than coenzyme Q10 are present together, a solution obtained after subjecting only oxidized coenzyme Q10 to reduction treatment by a known reduction method, or the like, can be used as a solution in the subsequent separation step.

In one or more embodiments of the present invention, as an organic solvent for use in a solution containing reduced coenzyme Q10, reduced demethoxy Q10, and reduced coenzyme Q9, it is preferred to use at least one organic solvent selected from the group consisting of alcohol, hydrocarbon, fatty acid ester, and nitrogen compound.

As the alcohol to be used as the organic solvent, regardless of whether cyclic alcohols or acyclic alcohols and, further, regardless of whether saturated alcohols or unsaturated alcohols, it is not particularly limited, but, in general, saturated alcohols are preferably used. For example, typically, a monohydric alcohol having 1 or more carbon atoms, in one embodiment, having 2 or more carbon atoms, typically a monohydric alcohol having 20 or less carbon atoms, in one embodiment, having 12 or less carbon atoms, in one embodiment, having 6 or less carbon atoms, in one embodiment, having 5 or less carbon atoms, in one embodiment, having 4 or less carbon atoms, in one embodiment, having 3 or less carbon atoms is preferred, or typically, a dihydric alcohol having 2 or more carbon atoms, typically having 5 or less carbon atoms is preferred, or a trihydric alcohol having 3 carbon atoms is preferred. For example, a monohydric alcohol having 1 to 20 carbon atoms, 1 to 12 carbon atoms, particularly 1 to 6 carbon atoms, more particularly 1 to 5 carbon atoms, still more particularly 1 to 4 carbon atoms, even more particularly 1 to 3 carbon atoms, and still further having 2 to 3 carbon atoms is preferred, or a dihydric alcohol having 2 to 5 carbon atoms is preferred, or a trihydric alcohol having 3 carbon atoms is preferred. Among these, a monohydric alcohol of 1 to 5 carbon atoms is an alcohol having high compatibility with water and is preferably used in the case of use as a mixed solvent with water.

Examples of the monohydric alcohols can include methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-1-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol, 4-methylcyclohexanol, and the like.

The monohydric alcohol is preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, and cyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, and neopentyl alcohol, more preferably methanol, ethanol, 1-propanol, 2-propanol, 1-butanol, 2-butanol, isobutyl alcohol, 2-methyl-1-butanol, and isopentyl alcohol, in particular, methanol, ethanol, 1-propanol, and 2-propanol, further preferably, ethanol, 1-propanol, and 2-propanol, and more preferably ethanol.

Examples of the dihydric alcohols include 1,2-ethanediol, 1,2-propanediol, 1,3-propanediol, 1,2-butanediol, 1,3-butanediol, 1,4-butanediol, 2,3-butanediol, 1,5-pentanediol, and the like. Preferably, the dihydric alcohol is 1,2-ethanediol, 1,2-propanediol, or 1,3-propanediol, more preferably, 1,2-ethanediol.

As a trihydric alcohol, glycerin or the like can preferably be used.

The hydrocarbon is not particularly limited, and examples thereof include an aliphatic hydrocarbon, an aromatic hydrocarbon, a halogenated hydrocarbon, and the like.

As the aliphatic hydrocarbon, regardless of whether cyclic aliphatic hydrocarbons or acyclic aliphatic hydrocarbons, and regardless of whether saturated aliphatic hydrocarbons or unsaturated aliphatic hydrocarbons, it is not particularly limited, but typically the aliphatic hydrocarbons having 3 or more carbon atoms, in one embodiment the aliphatic hydrocarbons having 5 or more carbon atoms, typically the aliphatic hydrocarbons having 20 or less carbon atoms, and in one embodiment the aliphatic hydrocarbons having 12 or less carbon atoms are used. The aliphatic hydrocarbon is not particularly limited regardless of whether cyclic aliphatic hydrocarbons or acyclic aliphatic hydrocarbons, and regardless of whether saturated aliphatic hydrocarbons or unsaturated aliphatic hydrocarbons, and typically the aliphatic hydrocarbons having 3 to 20 carbon atoms, preferably 5 to 12 carbon atoms, are used. Specific examples include propane, butane, isobutane, pentane, 2-methylbutane, cyclopentane, 2-pentene, hexane, 2-methylpentane, 2,2-dimethylbutane, 2,3-dimethylbutane, methylcyclopentane, cyclohexane, 1-hexene, cyclohexene, heptane, 2-methylhexane, 3-methylhexane, 2,3-dimethylpentane, 2,4-dimethylpentane, methylcyclohexane, 1-heptene, octane, 2,2,3-trimethylpentane, isooctane, ethylcyclohexane, 1-octene, nonane, 2,2,5-trimethylhexane, 1-nonene, decane, 1-decene, p-menthane, undecane, dodecane, and the like. Preferable examples are pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, nonane, 2,2,5-trimethylhexane, decane, dodecane, cyclopentane, methylcyclopentane, cyclohexane, methylcyclohexane, ethylcyclohexane, p-menthane, and the like. More preferable examples are pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, cyclopentane, methylcyclopentane, cyclohexane, methylcyclohexane, ethylcyclohexane, and the like, more preferable examples are pentane, hexane, cyclohexane, methylcyclohexane, and the like, more preferable examples are heptane, hexane, methylcyclohexane, and more preferable examples are heptane and hexane.

The aromatic hydrocarbon is not particularly limited, but typically the aromatic hydrocarbons having 6 or more carbon atoms, in one embodiment, the aromatic hydrocarbons having 7 or more carbon atoms, typically the aromatic hydrocarbons having 20 or less carbon atoms, in one embodiment, the aromatic hydrocarbons having 12 or less carbon atoms, and in one embodiment the aromatic hydrocarbons having 10 or less carbon atoms are used. The aromatic hydrocarbon is not particularly limited, but typically the aromatic hydrocarbons having 6 to 20 carbon atoms, preferably 6 to 12 carbon atoms, more preferably 7 to 10 carbon atoms are used. Specific examples include benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene, styrene, and the like.

The halogenated hydrocarbon is not particularly limited regardless of whether cyclic halogenated hydrocarbons or acyclic halogenated hydrocarbons and regardless of whether saturated halogenated hydrocarbons or unsaturated halogenated hydrocarbons, but acyclic ones are preferably used. Chlorinated hydrocarbons and fluorinated hydrocarbons are more preferred, and chlorinated hydrocarbons are further preferred.

As the halogenated hydrocarbon, preferably the halogenated hydrocarbons having 1 or more carbon atoms, preferably the halogenated hydrocarbons having 6 or less carbon atoms, more preferably the halogenated hydrocarbons having 4 or less carbon atoms, and more preferably the halogenated hydrocarbons having 2 or less carbon atoms are used. Further, as the halogenated hydrocarbon, preferably the halogenated hydrocarbons having 1 to 6 carbon atoms, more preferably the halogenated hydrocarbons having 1 to 4 carbon atoms, and more preferably the halogenated hydrocarbons having 1 to 2 carbon atoms are used. Specific examples include dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2-tetrachloroethane, pentachloroethane, hexachloroethane, 1,1-dichloroethylene, 1,2-dichloroethylene, trichloroethylene, tetrachloroethylene, 1,2-dichloropropane, 1,2,3-trichloropropane, chlorobenzene, and 1,1,1,2-tetrafluoroethane, and the like.

The fatty acid esters are not particularly limited, and examples thereof include propionic acid esters (propionate), acetic acid esters (acetate), formic acid esters (formate), and the like. Acetic acid esters and formic acid esters are preferred, and acetic acid esters are more preferred.

The ester group is not particularly limited, and examples thereof include an alkyl ester having 1 or more carbon atoms, an alkyl ester having 8 or less carbon atoms, an aralkyl ester having 1 or more carbon atoms, and, typically, an aralkyl ester having 8 or less carbon atoms, preferably having 6 or less carbon atoms, more preferably having 4 or less carbon atoms, and the like. The ester group is not particularly limited, and examples thereof include an alkyl ester having 1 to 8 carbon atoms, an aralkyl ester having 1 to 8 carbon atoms, and the like. The ester group is preferably an alkyl ester having 1 to 6 carbon atoms, and more preferably an alkyl ester having 1 to 4 carbon atoms.

Examples of the propionic acid esters include methyl propionate, ethyl propionate, butyl propionate, isopentyl propionate, and the like.

Examples of the acetic acid esters include methyl acetate, ethyl acetate, propyl acetate, isopropyl acetate, butyl acetate, isobutyl acetate, sec-butyl acetate, pentyl acetate, isopentyl acetate, sec-hexyl acetate, cyclohexyl acetate, benzyl acetate, and the like. Preferred examples include methyl acetate, ethyl acetate, propyl acetate, isopropyl acetate, butyl acetate, isobutyl acetate, and the like, and more preferred is ethyl acetate.

Examples of the formic acid esters include methyl formate, ethyl formate, propyl formate, isopropyl formate, butyl formate, isobutyl formate, sec-butyl formate, pentyl formate, and the like.

As a nitrogen compound, for example, a nitrile can be used. As for the nitrile, the nitrile is not particularly limited regardless of whether cyclic nitriles or acyclic nitriles, and regardless of whether saturated nitriles or unsaturated nitriles, but saturated nitriles are preferably used. Typically, nitriles having 2 or more carbon atoms, typically nitriles having 20 or less carbon atoms, preferably having 12 or less carbon atoms, more preferably having 8 or less carbon atoms, are used. Typically, nitriles having 2 to 20 carbon atoms, preferably having 2 to 12 carbon atoms, more preferably having 2 to 8 carbon atoms, are used. Specific examples of the nitrile include acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chloropropionitrile, bromopropionitrile, methoxyacetonitrile, methyl cyanoacetate, ethyl cyanoacetate, tolunitrile, benzonitrile, chlorobenzonitrile, bromobenzonitrile, cyanobenzoic acid, nitrobenzonitrile, anisnitrile, phthalonitrile, bromotolunitrile, methyl cyanobenzoate, methoxybenzonitrile, acetylbenzonitrile, naphthonitrile, biphenylcarbonitrile, phenylpropionitrile, phenylbutyronitrile, methyl phenylacetonitrile, diphenylacetonitrile, naphthylacetonitrile, nitrophenylacetonitrile, chlorobenzyl cyanide, cyclopropanecarbonitrile, cyclohexanecarbonitrile, cycloheptanecarbonitrile, phenylcyclohexanecarbonitrile, and tolylcyclohexanecarbonitrile. Preferable examples are acetonitrile, propionitrile, succinonitrile, butyronitrile, isobutyronitrile, valeronitrile, methyl cyanoacetate, ethyl cyanoacetate, benzonitrile, tolunitrile, or chloropropionitrile, more preferable examples are acetonitrile, propionitrile, butyronitrile, or isobutyronitrile, and a more preferable example is acetonitrile.

Examples of the nitrogen compounds other than the above nitrile include nitromethane, triethylamine, pyridine, formamide, N-methylformamide, N,N-dimethylformamide, N,N-dimethylacetamide, N-methylpyrrolidone, and the like.

Among the above organic solvents, an alcohol or a hydrocarbon is preferred, and an alcohol is particularly preferred.

In one or more embodiments of the present invention, as the organic solvent for use in a solution that contains reduced coenzyme Q10 and the like, the ones exemplified above may be used alone, and in order to improve conditions that affect crystal precipitation conditions such as the solubility of each component of reduced coenzyme Q10 and the like, crystal precipitation concentration, yield, slurry physical properties, and crystal physical properties, two or more solvents may also be mixed in an appropriate proportion according to the properties of each solvent and used.

Further, as long as the use of at least one organic solvent selected from the group consisting of the above alcohol, hydrocarbon, fatty acid ester and nitrogen compound is made, the auxiliary use of organic solvents other than those is acceptable. Furthermore, in one or more embodiments of the present invention, “organic solvent” includes organic solvents containing water. When an organic solvent containing water is used, the water content in the organic solvent, based on the total amount of the organic solvent containing water, is preferably 0.01 wt % or more, more preferably 0.1 wt % or more, still more preferably 0.5 wt % or more, preferably 50 wt % or less, more preferably 40 wt % or less, still more preferably 34 wt % or less, and particularly preferably 15 wt % or less. When an organic solvent containing water is used, the water content in the organic solvent, based on the total weight of the organic solvent containing water, is preferably 0.01 to 50 wt %, more preferably 0.1 to 40 wt %, still more preferably 0.5 to 34 wt %, and particularly preferably 0.5 to 15 wt %. When the water content exceeds 50 wt %, there are cases where separation of the organic solvent and water occurs, the solubility of reduced coenzyme Q10 and the like in the organic solvent is greatly reduced, and the crystallization of reduced coenzyme Q10 and the like for the purpose becomes difficult. In the case of use of an organic solvent containing water, it is preferred that the water and the organic solvent are in a homogeneous system, and, from this viewpoint as well, alcohols such as ethanol and fatty acid esters such as ethyl acetate are preferably selected as the organic solvent.

In one or more embodiments of the present invention, the organic solvent is a monohydric alcohol typically having 1 or more carbon atoms and typically having 5 or less carbon atoms that contains 15 wt % or less of water or contains no water, and, more preferably, is a monohydric alcohol typically having 1 or more carbon atoms and typically having 5 or less carbon atoms that contains 8 wt % or less of water or contains no water. In one or more embodiments of the present invention, the organic solvent is a monohydric alcohol having 1 to 5 carbon atoms that contains 15 wt % or less of water or contains no water, more preferably, the organic solvent is a monohydric alcohol having 1 to 5 carbon atoms that contains 8 wt % or less of water or contains no water, more preferably, the organic solvent is ethanol that contains 8 wt % or less of water or contains no water, and further preferably, the organic solvent is ethanol that contains 0.1 wt % or more and 8 wt % or less of water.

In the solution containing the organic solvent, the concentrations of reduced coenzyme Q10, reduced demethoxy Q10, and reduced coenzyme Q9 can be appropriately adjusted according to the organic solvent for use and are not particularly limited. For example, the concentration of the total of reduced coenzyme Q10, reduced demethoxy Q10, and reduced coenzyme Q9 in a solution is 80 wt % or less, preferably, 70 wt % or less, more preferably 60 wt % or less, further preferably 50 wt % or less, and particularly preferably 40 wt % or less. Also, from the standpoint of production efficiency, it is preferred that the concentration of reduced coenzyme Q10 and the like in the solution be adjusted to a relatively high concentration, for example, preferably 1 wt % or more, more preferably 5 wt % or more, and particularly preferably 10 wt % or more. That is, the total concentration of reduced coenzyme Q10, reduced demethoxy Q10, and reduced coenzyme Q9 in the solution can be, with respect to the total weight of the solution, for example 1 to 80 wt %, preferably 5 to 70 wt %, more preferably 10 to 60 wt %, even preferably 10 to 50 wt %, and particularly preferably 10 to 40 wt %.

The solution to be used in the step of precipitation of the composition in solid form, for example, the step of adding a seed crystal to cause precipitation of the crystal is a supersaturated solution with dissolution at a concentration equal to or higher than the saturation concentration of each component such as reduced coenzyme Q10, more preferably at a temperature of 32° C. or higher, more preferably at a temperature of 43° C. or lower. The solution to be used in the step of precipitation of the composition in solid form, for example, the step of adding a seed crystal to cause precipitation of the crystal is, more preferably, a supersaturated solution with dissolution at a concentration equal to or higher than the saturation concentration of each component such as reduced coenzyme Q10 at a temperature within the range of 32 to 43° C. Such a supersaturated solution can be prepared by heating a raw material mixture that contains the organic solvent and the respective components such as reduced coenzyme Q10 to a temperature of 42° C. or higher, 45° C. or higher, more preferably 49° C. or higher, further preferably 70° C. or lower, and more preferably 55° C. or lower to effect dissolution of the respective components such as reduced coenzyme Q10, and by cooling the solution after the heating to a temperature lower than the heating temperature and a temperature within the range of 32 to 43° C.

Particularly, in the case where the organic solvent is a monohydric alcohol having 1 to 5 carbon atoms (preferably ethanol) that contains 8 wt % or less of water or contains no water, the total concentration of reduced coenzyme Q10, reduced demethoxy Q10, and reduced coenzyme Q9 in the solution is preferably 5 wt % or more, particularly preferably 10 wt % or more, preferably 50 wt % or less, more preferably 40 wt % or less, more preferably 25 wt % or less, and particularly preferably 20 wt % or less. When the concentration is within the range above, a supersaturated solution of reduced coenzyme Q10 and the like is easy to prepare, and by addition of a seed crystal, precipitation of the crystal is easy.

In one or more embodiments of the present invention, a crystal of reduced coenzyme Q10 is added as a seed crystal to the solution containing the organic solvent and reduced coenzyme Q10, reduced demethoxy Q10 and reduced coenzyme Q9, thereby preparing a mixed liquid to be subjected to the crystal precipitation step. The addition amount (seed crystal addition amount) of the reduced coenzyme Q10 crystal that is the seed crystal, if added, is not particularly limited, but is, with respect to the total weight of reduced coenzyme Q10 in the solution before addition of the seed crystal, preferably 0.001 wt % or more, more preferably 0.005 wt % or more, particularly preferably 0.01 wt % or more, preferably 30 wt % or less, more preferably 20 wt % or less, and particularly preferably 5 wt % or less. The addition amount (seed crystal addition amount) of the reduced coenzyme Q10 crystal that is the seed crystal, if added, is not particularly limited, but is, with respect to the total weight of reduced coenzyme Q10 in the solution before addition of the seed crystal, preferably 0.001 to 30 wt %, more preferably 0.005 to 20 wt %, and particularly preferably 0.01 to 5 wt %. Note that the reduced coenzyme Q10 crystal for use as a seed crystal is preferably one that includes the reduced coenzyme Q10 crystal of the Form I type, but it is also acceptable if the reduced coenzyme Q10 crystal of the Form II type or an amorphous form of the reduced coenzyme Q10 is included. A higher purity of the Form I type reduced coenzyme Q10 crystal is preferred, and, for example, the use of a seed crystal containing the Form I type reduced coenzyme Q10 crystal at a concentration of 50 wt % or more, preferably 75 wt % or more, more preferably 80 wt % or more, and more preferably 90 wt % or more, is preferred.

Furthermore, in the present embodiment, the temperature of the solution at the time of addition of the seed crystal is preferably 25° C. or higher, and is preferably 43° C. or lower. In one or more embodiments of the present invention, it is preferred that the temperature of the solution at the time of addition of the seed crystal is within the range of 25 to 43° C. More preferably, the temperature of the solution at the time of addition of the seed crystal is 27° C. or higher, and, more preferably, is 29° C. or higher. Further, it is preferred that the upper limit be 41° C. or lower. If the temperature of the solution at the time of addition of the seed crystal exceeds 43° C., there are cases where dissolution of the added seed crystal occurs and precipitation of the crystal does not occur. Further, if a seed crystal is added to a solution at the temperature of less than 25° C., the proportion of the crystal in the reduced coenzyme Q10 obtained by precipitation may become low, or precipitation of the crystal may not occur.

Next, one or more preferred embodiments of the crystal precipitation step that causes precipitation of a reduced coenzyme Q10 crystal in the mixed liquid will be described. If the seed crystal is added, as long as the temperature of the solution at the time of addition of the seed crystal is within the above range, the temperature of the crystal precipitation step after adding the seed crystal is not particularly limited, but the step preferably includes maintaining, for 1 hour or more after adding the seed crystal, the temperature of the mixed liquid at a temperature preferably −1° C. or higher, preferably 41° C. or lower. If the seed crystal is added, as long as the temperature of the solution at the time of addition of the seed crystal is within the above range, the temperature of the crystal precipitation step after adding the seed crystal is not particularly limited, but the step preferably includes maintaining, for 1 hour or more after adding the seed crystal, the temperature of the mixed liquid, preferably at −1° C. or higher, more preferably within the range of −1 to 41° C. The time to maintain the mixed liquid in the temperature range is not particularly limited, but is, preferably 1 hour or more, preferably 2 hours or more, more preferably 4 hours or more, and particularly preferably 10 hours or more. That is, the time to maintain the mixed liquid in the temperature range is preferably within the range of, for example, 1 to 24 hours. The upper limit of the time to maintain the mixed liquid in the temperature range is not particularly limited, but a sufficient effect can be obtained in about 24 hours. In this case, for example, a constant temperature in the range of −1 to 41° C. may be maintained, or, for example, in cooling crystallization or the like, after addition of the seed crystal, the mixed liquid may be allowed to reach −1° C. while being gradually cooled. Further, throughout the crystal precipitation step, it may be permissible to maintain a temperature of −1 to 41° C., or, after maintaining a temperature of −1 to 41° C. for, for example, a time of 1 hour or more, thereafter to perform cooling.

The endpoint temperature of the crystal precipitation step is not particularly limited, but, from the viewpoint of an increase in recovery amount, the crystal precipitation step is preferably carried out at an endpoint temperature of 35° C. or lower, more preferably 30° C. or lower, and particularly 25° C. or lower. The lower limit is the solidification temperature of the system, but is preferably −10° C. or higher, more preferably −1° C. or higher. Specifically, the endpoint temperature of the precipitation step may be set within the range of −10 to 35° C., preferably −1 to 30° C., more preferably −1 to 25° C.

In the crystal precipitation step, it is preferred to control the precipitation amount of crystal per unit time and thereby control the formation of supersaturation. A preferred precipitation amount per unit time is, for example, equal to or less than a rate to precipitate about 50% of the total precipitation amount per unit time (that is, at most 50% amount/hour), and preferably equal to or less than a rate to precipitate 25% of the total precipitation amount per unit time (that is, at most 25% amount/hour).

In one or more embodiments of the present invention, the crystal precipitation step includes lowering the temperature of the mixed liquid over time, that is, a cooling crystallization step. In cooling crystallization, the mixed liquid is cooled to lower the solubility in the liquid phase of each component such as reduced coenzyme Q10 and to promote crystallization. The step of cooling the mixed liquid is preferably performed successively after the above step of maintaining the mixed liquid at a temperature of 25° C. or higher for a constant time to cause precipitation of the crystal capable of precipitation at this temperature. Lowering the temperature of the mixed liquid over time includes continuously lowering the temperature of the mixed liquid with elapsed time, stepwise lowering the temperature of the mixed liquid, and combinations thereof. When lowering the temperature of the mixed liquid over time, the cooling rate is not particularly limited, but, for example, the cooling rate is such that the temperature drop per hour is 30° C. or lower, preferably 25° C. or lower, more preferably 20° C. or lower, more preferably 15° C. or lower, more preferably 10° C. or lower, more preferably 0.5° C. or higher, and more preferably 1° C. or higher. The cooling rate in the case of lowering the temperature of the mixed liquid over time may be a constant, or may be subject to change. In particular, if the cooling rate is such that, as the temperature of the mixed liquid decreases, the cooling rate increases continuously or stepwise, that is, so that the magnitude of the temperature decrease per hour becomes larger, efficient crystallization of reduced coenzyme Q10, whose residual amount in the liquid phase decreases as the temperature of the mixed liquid decreases, can be carried out. For example, until the temperature of the mixed liquid reaches 25° C., cool the mixed liquid at a rate at which the temperature decrease per hour is preferably 5° C. or lower, more preferably 3° C. or lower, and, in a stage in which the mixed liquid is further cooled to a temperature of less than 25° C., it is possible to cool the mixed liquid at a rate at which the temperature decrease per hour is preferably 6° C. or higher, more preferably 8° C. or higher. In one or more embodiments of the present invention, the endpoint temperature that is reached by lowering the temperature of the mixed liquid over time is preferably 25° C. or lower, more preferably 20° C. or lower, more preferably 10° C. or lower, more preferably 7° C. or lower, and more preferably 5° C. or lower. The lower limit of the endpoint temperature is the solidification temperature of the system of the mixed liquid, and is preferably −10° C. or higher, preferably −7° C. or higher, preferably −5° C. or higher, and more preferably −1° C. or higher. For example, the endpoint temperature may be within the range of −10 to 25° C., preferably −10 to 20° C., more preferably −7 to 10° C., still more preferably −5 to 7° C., and particularly preferably −5 to 5° C.

It is preferred to carry out the precipitation of the crystal while causing forced flow of the mixed liquid after addition of the seed crystal. In order to suppress the formation of supersaturation and to carry out nucleation and crystal growth smoothly, or from the viewpoint of higher quality, as the agitation power requirement per unit volume, it is good to impart the mixed liquid a flowing of typically about 0.005 kW/m³ or more, preferably 0.01 kW/m³ or more, more preferably 0.015 kW/m³ or more, and still more preferably 0.03 kW/m³ or more. The above forced flow is typically imparted by rotation of a stirring blade, but if the above flowing is obtained, it is not necessarily required to use a stirring blade, and, for example, a method using circulation of a mixed liquid or the like may be utilized.

In the method or the production method of the oral composition of one or more embodiments of the present invention, the step of separating, from the solution, a composition in solid form containing reduced coenzyme Q10, reduced demethoxy Q10, and reduced coenzyme Q9 is not particularly limited, and, in addition to the above cooling crystallization, methods such as poor solvent crystallization and concentration crystallization can be utilized. It is preferred that it be cooling crystallization, or a method combining cooling crystallization with another crystallization method. Poor solvent crystallization is a method that, by mixing a poor solvent with the mixed liquid, lowers the solubility and causes crystallization of reduced coenzyme Q10. Here, the term “poor solvent” means a solvent in which either dissolution of reduced coenzyme Q10 hardly occurs or dissolution of reduced coenzyme Q10 does not occur at all. It is preferred that the poor solvent mutually dissolve with an organic solvent used in a solution that contains reduced coenzyme Q10 and the like.

As methods to mix a poor solvent with a solution, one may add the poor solvent to the solution, or one may add the solution to the poor solvent. Examples of other crystallization methods to be combined with cooling crystallization include, in addition to the above-described poor solvent crystallization, for example, concentration crystallization that causes precipitation of the crystal by concentrating a solution, and the like.

The composition in solid form, for example, the crystal or crystalline solid, of reduced coenzyme Q10 obtained by the above method can be recovered, for example, by a conventionally known method, through the steps of solid-liquid separation and drying. For example, for solid-liquid separation, the use of pressure filtration, filtration by centrifugation, and the like is possible. Additionally, the crystal or the crystalline solid after drying can also be subjected to pulverization and classification as necessary, and recovered.

As one of the more preferred aspects, by performing, under heating, the drying of the composition in solid form, for example, the crystal or crystalline solid, of reduced coenzyme Q10 after the above solid-liquid separation, it is possible to improve the content ratio of the reduced coenzyme Q10 crystal. For this purpose, the drying temperature is preferably 33° C. or higher, more preferably 34° C. or higher, and still more preferably 35° C. or higher. The upper limit is typically 46° C. or lower, preferably 45° C. or lower. For example, the drying temperature can be set within the range of 33 to 46° C., preferably 34 to 46° C., and more preferably within the range of 35 to 45° C. When below 33° C., drying proceeds, but the content ratio of the reduced coenzyme Q10 crystal hardly increases. In addition, when exceeding 46° C., during drying the reduced coenzyme Q10 crystal may undergo melting. Also, the heating time in the case of performing drying under the above temperature conditions is not particularly limited, but is, preferably 4 hours or more, preferably 10 hours or more, and more preferably 20 hours or more.

Note that, in the crystal precipitation step, when the content ratio of the reduced coenzyme Q10 crystal for the purpose has already been achieved, it is not limited to the above, and, for example, the drying may be carried out at 20° C. or higher, preferably 25° C. or higher, more preferably 30° C. or higher.

In addition, In the method or the production method of the oral composition of one or more embodiments of the present invention, it is preferred that each step, specifically the mixed liquid formation step, the crystal precipitation step, recovery steps such as solid-liquid separation and drying, described above, and other subsequent processing steps and the like, be carried out under a deoxygenated atmosphere. A deoxygenated atmosphere can be achieved by substitution of the atmosphere with an inert gas, by reduced pressure, by boiling, or by combining these. At least, substitution of the atmosphere with an inert gas, i.e., using an inert gas atmosphere, is preferred. Examples of the above inert gas include nitrogen gas, helium gas, argon gas, hydrogen gas, carbon dioxide gas, and the like, and nitrogen gas is preferred.

Whether the reduced coenzyme Q10 crystal of the Form I type or Form II type are contained in the obtained composition in solid form, for example, the crystal or crystalline solid, of reduced coenzyme Q10 and the content ratio thereof, can be determined, for example, by measuring with a differential scanning calorimeter (DSC).

The Form II type reduced coenzyme Q10 crystal, when measured by DSC at a heating rate of 1° C./min, shows an endothermic peak in the vicinity of 52±2° C., and the Form I type reduced coenzyme Q10 crystal, under the same conditions, shows an endothermic peak in the vicinity of 48±1° C. Even when a Form II type reduced coenzyme Q10 crystal is in a state mixed with a conventional Form I type reduced coenzyme Q10 crystal or its crystalline solid, it is possible to make a determination of the presence or absence of the Form II type reduced coenzyme Q10 crystal and its content ratio by the presence or absence of the peak around 52±2° C., the height of that endothermic peak, and the ratio of the heats of absorption.

In a case where the obtained composition in solid form of reduced coenzyme Q10 contains Form I crystal, when Form II type reduced coenzyme Q10 crystal in an amount of 10 wt % with respect to the Form I crystal contained in the composition and 99.5% ethanol are added to the composition, and the mixture is stirred and then allowed to stand at 37° C. for a time of 6 hours, the proportion of the Form II crystal in the mixture, in a measurement by a differential scanning calorimeter (DSC), as the proportion (%) of the endothermic peak area of the Form II crystal to the total of the endothermic peak area of the Form I crystal and the endothermic peak area of the Form II crystal, is typically 25% or less, in one embodiment, 24% or less, in one embodiment, 23% or less, in one embodiment, 22% or less, in one embodiment, 21% or less, in one embodiment, 20% or less, in one embodiment, 19% or less, in one embodiment, 18% or less, in one embodiment, 17% or less, in one embodiment, 16% or less, in one embodiment, 15% or less, in one embodiment, 14% or less, in one embodiment, 13% or less, in one embodiment, 12% or less, in one embodiment, 11% or less, in one embodiment, 10% or less, in one embodiment, 9% or less, and in one embodiment, 8% or less. The lower limit value of the proportion is not limited, but the proportion is typically 1% or more.

In a case where the obtained composition in solid form of reduced coenzyme Q10 contains Form I crystal, when Form II type reduced coenzyme Q10 crystal in an amount of 10 wt % with respect to the Form I crystal contained in the composition and 99.5% ethanol are added to the composition, and the mixture is stirred and then allowed to stand at 37° C. for a time of 6 hours, the fact that the proportion of Form II crystal in the mixture is small means that the transition rate from Form I crystal to Form II crystal of the reduced coenzyme Q10 composition according to one or more embodiments of the present invention is small, and therefore means that the polymorphic stability of the crystal of the reduced coenzyme Q10 composition according to one or more embodiments of the present invention is high. When the polymorphic stability of the crystal of the reduced coenzyme Q10 composition is high, quality change due to transition of crystal polymorphism is less likely to occur, and it is more preferred in terms of quality control.

When forming the coexisting state in the composition in solid form or producing the oral composition through a step of separation of reduced coenzyme Q10, reduced demethoxy Q10, and reduced coenzyme Q9 from a solution, for example, in accordance with each form such as powder, granules, tablets, hard capsules, or the like, it is possible to form or produce the composition by conventionally known steps.

For example, one can form or produce the coexisting state or the oral composition of reduced coenzyme Q10 in powder form by mixing, as powder raw materials, reduced coenzyme Q10 and emulsified starch and, if necessary, substances other than the reduced coenzyme Q10 and the emulsified starch such as antioxidants, emulsifiers, binders, and excipients.

Further, one can form or produce the coexisting state or the oral composition of reduced coenzyme Q10 in granule form, for example, by mixing, as powder raw materials, reduced coenzyme Q10 and emulsified starch, a binder, and, as necessary, substances other than the reduced coenzyme Q10 and the emulsified starch such as an antioxidant and an emulsifier, adding water to the mixture, and going through steps of granulation, drying, classification, and pulverization.

Furthermore, one can form or produce the coexisting state or the oral composition of reduced coenzyme Q10 in tablet form by tableting the reduced coenzyme Q10 composition in the powder or granule form by a conventional method.

One can form or produce the coexisting state or the oral composition of reduced coenzyme Q10 in hard capsule form by filling a hard capsule with the reduced coenzyme Q10 composition in the powder or granule form by a conventional method.

The granulation method is not particularly limited, and, for example, may be appropriately selected from methods such as an extrusion granulation method, a stirring granulation method, a tumbling granulation method (a rotating granulation method), a dry granulation method, a compression granulation method, a powder bonding method, a fluidized bed granulation method, a coacervation method, a spray-drying method, a cold spray method, an evaporation method, and an in-liquid curing method. The granulation method is preferably carried out by a granulation method in which pressure is applied (for example, the extrusion granulation method, the stirring granulation method, the tumbling granulation method, the dry granulation method, the compression granulation method, the powder bonding method, and the like) or by the fluidized bed granulation method, more preferably by the extrusion granulation method, the stirring granulation method, or the fluidized bed granulation method, and particularly preferably by the extrusion granulation method or the stirring granulation method.

In one or more embodiments of the present invention, when the reduced coenzyme Q10 crystal is caused to coexist or is contained in the coexisting state or the oral composition, in the granulation process the reduced coenzyme Q10 crystal is not completely dissolved or melted, and is mixed and granulated while basically maintaining the crystal state.

The components other than the liquid binder of the mixture to be subjected to granulation may be in the form of a powder mixture. The characteristics of the respective components other than the liquid binder of the mixture are as described above.

Examples of the liquid binder include water, ethanol, and the like. Additionally, the above-described binder may be caused to undergo dissolution in a liquid such as water or ethanol and used as a liquid binder. As the liquid binder, water is particularly preferred.

Granulation can be carried out by appropriately setting conditions so that a composition in solid form having dimensions according to the application for the purpose is obtained.

It is preferred to further include a drying step of drying, after granulation, the reduced coenzyme Q10 composition in solid form and removing, by drying, volatile components derived from each raw material component and the liquid binder. Additionally, as necessary, by sieving or the like, it is possible to perform separation and recovery of a composition in solid form having a desired particle size.

One can form or produce the coexisting state or the oral composition of reduced coenzyme Q10 in tablet form by tableting a composition in powder form, or a composition in granule form obtained through steps such as granulation and drying, but the tableting method is not particularly limited. For example, it is possible to appropriately select from methods such as the direct compression method, the semi-direct compression method, a dry granulation compression method, and a wet granulation compression method. The tableting method is preferably performed by the direct compression method or the semi-direct compression method, and the direct compression method is more preferred.

The coexisting state of reduced coenzyme Q10 according to one or more embodiments of the present invention may be formed in general food and beverage, and the oral composition of reduced coenzyme Q10 according to one or more embodiments of the present invention can also be added to general food and beverage. The food and beverage in which the coexisting state of reduced coenzyme Q10 is formed, or to which the oral composition of reduced coenzyme Q10 is added is not particularly limited, and, for example, the coexisting state of reduced coenzyme Q10 or the oral composition of reduced coenzyme Q10 can be appropriately added to and used in beverages such as milk beverages, soft drinks, sports drinks, nutritional drinks, beauty drinks, liquid nutritional preparations, and the like, confectionery such as chewing gum, chocolate, candies, jelly, cakes, biscuits, crackers, and the like, frozen desserts such as ice cream, ice confections, and the like, noodles such as udon, Chinese noodles, spaghetti, instant noodles, and the like, fish paste products such as kamaboko, chikuwa, hanpen, and the like, seasonings such as dressings, mayonnaise, sauces, and the like, bread, ham, rice porridge, cooked rice, soups, various retort foods, various frozen foods, and the like. A food or beverage forming the coexisting state or containing the oral composition of reduced coenzyme Q10 according to one or more embodiments of the present invention can be used in applications such as so-called health food, supplement, functional food, Food with Function Claims, nutritional supplement, Food for Specified Health Uses, Nutrient Function Food, nursing care food, Smile Care Food, mastication and swallowing aid food, thick flowing food, and food for patients and the like. Alternatively, it can also be used as other food forms. Furthermore, the coexisting state of reduced coenzyme Q10 according to one or more embodiments of the present invention can also be formed in general pharmaceuticals, and the oral composition of reduced coenzyme Q10 according to one or more embodiments of the present invention can also be added to general pharmaceuticals. Furthermore, it can also be used for pet food, livestock feed, and the like.

The first aspect, the second aspect, and the third aspect in one or more embodiments of the present invention are each independent, however, the definitions of terms, provisions, ranges, embodiments, and the like in the first aspect, the second aspect, or the third aspect in one or more embodiments of the present invention may be replaced with or may be combined with the definitions of terms, provisions, ranges, embodiments, and the like corresponding to another aspect in the present disclosure.

The present specification incorporates the disclosure of Japanese Patent Application No. 2025-058032, which serves as the basis for the priority of the present application.

Further, the present specification incorporates the disclosure of Japanese Patent Application No. 2025-058008, which serves as the basis for the priority of the present application.

Furthermore, the present specification incorporates the disclosure of Japanese Patent Application No. 2025-058139, which serves as the basis for the priority of the present application.

### EXAMPLE

Hereinafter, one or more embodiments of the present invention will be described in further detail by examples, but, the present invention is not limited to these examples. It should be noted that each experimental example is an independent experiment.

### Experiment 1

As raw materials for the reduced coenzyme Q10 composition, the following Compositions A to C were used.

Composition A: a composition containing oxidized coenzyme Q10 (QX), oxidized coenzyme Q9 (Q9), oxidized coenzyme Q11 (Q1l), oxidized demethoxy Q10 (D), and decaprenylphenol (oxidized coenzyme Q10 content in the composition: 96.2 wt %)

Composition B: a composition containing oxidized coenzyme Q10 (QX), oxidized coenzyme Q9 (Q9), oxidized coenzyme Q11 (Q1l), oxidized demethoxy Q10 (D), and decaprenylphenol (oxidized coenzyme Q10 content in the composition: 97.7 wt %).

Composition C: a composition containing oxidized coenzyme Q10 (QX), oxidized coenzyme Q9 (Q9), oxidized coenzyme Q11 (Q1l), and oxidized demethoxy Q10 (D) (oxidized coenzyme Q10 content in the composition: 97.9 wt %).

Note that, with respect to Composition A, Composition B, and Composition C, in the chromatogram obtained by high performance liquid chromatography under the following conditions:
a column: YMC-Pack Pro C18 (manufactured by YMC) 150 mm (length) 4.6 mm (inner diameter),
a mobile phase composition: CH₃OH:C₆H₁₄=9:1 (v/v),
a mobile phase flow rate: 1 mL/min, and
a detection wavelength: 275 nm
   the peak area ratios of the respective components are as follows.

TABLE 4

| | D/ | Decaprenyl/ | D/ | Decaprenyl/ | Q11/ |
|---|---|---|---|---|---|
| Peak Area | Q9 | Q9 | Q11 | Q11 | Q9 |
| Composition A | 0.69 | 0.15 | 0.63 | 0.13 | 1.10 |
| Composition B | 1.51 | 0.47 | 0.66 | 0.21 | 2.28 |
| Composition C | 0.12 | 0.00 | 0.50 | 0.00 | 0.23 |

| Peak Area | Q9/QX | D/QX | Decaprenyl/QX | Q11/QX |
|---|---|---|---|---|
| Composition A | 0.0020 | 0.0014 | 0.00029 | 0.0022 |
| Composition B | 0.0012 | 0.0018 | 0.00056 | 0.0027 |
| Composition C | 0.0027 | 0.00031 | 0.00 | 0.00062 |

### Production Example 1

Into 1200 g of ethanol, 300 g of Composition A and 122 g of ascorbic acid were added, the mixture was stirred at 79° C., and a reduction reaction was carried out. After 20 hours from the start of the reduction reaction, cooling was performed to 50° C. To 900 g of the ethanol solution after cooling (containing reduced coenzyme Q10), 800 g of ethanol was added, and the mixture was further cooled to 33° C.

After adding a Form I type reduced coenzyme Q10 crystal as a seed crystal to the cooled ethanol solution, and cooling to −1° C., Composition a in solid form containing Form I type reduced coenzyme Q10 was obtained.

### Production Example 2

Except that Composition A in Production Example 1 was changed to Composition B, it was carried out in the same manner as in Production Example 1, and Composition b in solid form containing Form I type reduced coenzyme Q10 was obtained.

### Production Example 3

Except that Composition A in Production Example 1 was changed to Composition C, it was carried out in the same manner as in Production Example 1, and Composition c in solid form containing Form I type reduced coenzyme Q10 was obtained.

### (Experimental Example 1) Component Analysis

Component analysis by high performance liquid chromatography was performed on Compositions a, b, and c obtained in Production Examples 1 to 3. Tables 5 to 8 show the results. The respective numerical values in Tables 5 to 8 represent, as calculated from the chromatogram of each composition obtained by high performance liquid chromatography in accordance with Table 1, the peak area ratio of reduced demethoxy Q10 (RD) to reduced coenzyme Q9 (RQ9) (RD/RQ9), the peak area ratio of decaprenylphenol (decaprenyl) to reduced coenzyme Q9 (RQ9) (decaprenyl/RQ9), the peak area ratio of reduced demethoxy Q10 (RD) to reduced coenzyme Q11 (RQ11) (RD/RQ11), the peak area ratio of decaprenylphenol (decaprenyl) to reduced coenzyme Q11 (RQ11) (decaprenyl/RQ11), the peak area ratio of reduced coenzyme Q11 (RQ11) to reduced coenzyme Q9 (RQ9) (RQ11/RQ9), the peak area ratio of decaprenylphenol (decaprenyl) to reduced demethoxy Q10 (RD) (decaprenyl/RD), the peak area ratio of reduced coenzyme Q9 (RQ9) to reduced coenzyme Q10 (QH) (RQ9/QH), the peak area ratio of reduced demethoxy Q10 (RD) to reduced coenzyme Q10 (QH) (RD/QH), the peak area ratio of decaprenylphenol (decaprenyl) to reduced coenzyme Q10 (QH) (decaprenyl/QH), the peak area ratio of reduced coenzyme Q11 (RQ11) to reduced coenzyme Q10 (QH) (RQ11/QH), the peak area ratio of the total of reduced demethoxy Q10 (RD) and reduced coenzyme Q9 (RQ9) to reduced coenzyme Q10 (QH) ((RD+RQ9)/QH), and the peak area ratio of the total of decaprenylphenol (decaprenyl) and reduced coenzyme Q11 (RQ11) to reduced coenzyme Q10 (QH) ((decaprenyl+RQ11)/QH). In addition, the conditions for high performance liquid chromatography are as follows. (HPLC conditions)
a column: YMC-Pack Pro C18 (manufactured by YMC) 150 mm (length) 4.6 mm (inner diameter),
a mobile phase composition: CH₃OH:C₆H₁₄=9:1 (v/v),
a mobile phase flow rate: 1 ml/min, and
a detection wavelength: 190 nm

TABLE 5

| | RD/RQ9 | Decaprenyl/RQ9 | RD/RQ11 |
|---|---|---|---|
| Composition a | 1.00 | 1.44 | 0.30 |
| Composition b | 2.02 | 3.68 | 0.25 |
| Composition c | 0.17 | 0 | 0.18 |

TABLE 6

| | Decaprenyl/RQ11 | RQ11/RQ9 | Decaprenyl/RD |
|---|---|---|---|
| Composition a | 0.43 | 3.37 | 1.44 |
| Composition b | 0.46 | 8.03 | 1.82 |
| Composition c | 0 | 0.95 | 0 |

TABLE 7

| | RQ9/QH | RD/QH | Decaprenyl/QH |
|---|---|---|---|
| Composition a | 0.0049 | 0.0049 | 0.0071 |
| Composition b | 0.0026 | 0.0053 | 0.0096 |
| Composition c | 0.0056 | 0.00096 | 0 |

TABLE 8

| | RQ11/ | (RD + | (Decaprenyl + |
|---|---|---|---|
| | QH | RQ9)/QH | RQ11)/QH |
| Composition a | 0.017 | 0.0098 | 0.024 |
| Composition b | 0.021 | 0.0079 | 0.031 |
| Composition c | 0.0054 | 0.0066 | 0.0054 |

As shown in Table 5, in Composition a and Composition b, the ratio (RD/RQ9) of the peak area of reduced demethoxy Q10 (RD) to the peak area of reduced coenzyme Q9 was 0.3 or more. In particular, in Composition a, the value of RD/RQ9 was 1.0 or more, and in Composition b, the value of RD/RQ9 was 2.0 or more. On the other hand, in Composition c, the peak area of reduced demethoxy Q10 was smaller than the peak area of reduced coenzyme Q9, and the value of RD/RQ9 was less than 0.3.

Furthermore, as shown in Tables 5 to 7, decaprenylphenol was contained only in Compositions a and b, and was not contained in Composition c.

### (Experimental Example 2) Crystal Polymorphic Stability Test

After 0.5 g of Form II type reduced coenzyme Q10 crystal was added to 5.0 g of Composition a, Composition b, or Composition c obtained in Production Examples 1 to 3 and containing Form I type reduced coenzyme Q10 crystal, 2 g of 99.5% ethanol was added and the mixture was stirred. After stirring, nitrogen substitution was performed, and it was allowed to stand at 37° C. for 6 hours. While being allowed to stand, differential scanning calorimetry of the samples obtained by sampling at time intervals of 2 hours was performed, and the endothermic peak area due to melting of the Form I crystal and the endothermic peak area due to melting of the Form I crystal were determined. The proportion (%) of the endothermic peak area of the Form II crystal to the total of the endothermic peak area of the Form I crystal and the endothermic peak area of the Form II crystal was calculated as the ratio of the Form II crystal. The results are shown in Table 9.

TABLE 9

| | Composition a | Composition b | Composition c |
|---|---|---|---|
| 2 hr | 5.8 | 3.3 | 6.1 |
| 4 hr | 7.3 | 4.4 | 10.0 |
| 6 hr | 5.8 | 3.8 | 19.9 |

* Numerical values in table indicate Form II type ratio (%).

As shown in Table 9, Composition a and Composition b having an RD/RQ9 value of 1.0 or more had a small change in the ratio of Form II crystal until 6 hours after the start of standing. From this, it was suggested that the stability of the Form I type reduced coenzyme Q10 crystal contained in Composition a and Composition b is high, and that it is unlikely to undergo transition to Form II crystal. On the other hand, in Composition c, with elapsed time, the ratio of Form II crystal increased, and the stability of the Form I type reduced coenzyme Q10 crystal was inferior compared with Composition a and Composition b.

### (Experimental Example 3) Particle Size Distribution Measurement

For Compositions a, b, and c obtained in Production Examples 1 to 3, in wet analysis, the particle size distribution on a volume basis was measured by a laser diffraction/scattering particle size distribution analyzer (LA-960, manufactured by HORIBA, Ltd.). The result is shown in the FIGURE.

From the obtained particle size distribution, the average particle diameter, and the particle diameter of particles corresponding to a cumulative percentage of 10% (hereinafter referred to as D10), the particle diameter of particles corresponding to a cumulative percentage of 50% (hereinafter referred to as D50), and the particle diameter of particles corresponding to a cumulative percentage of 90% (hereinafter referred to as D90), in the cumulative particle size distribution were obtained. The results are shown in Table 10.

TABLE 10

| | D10 | D50 | D90 | Average Particle |
|---|---|---|---|---|
| Sample | (μm) | (μm) | (μm) | Diameter (μm) |
| Composition c | 22 | 90 | 223 | 110 |
| Composition b | 16 | 52 | 223 | 87 |
| Composition a | 17 | 53 | 149 | 70 |

Composition b exhibited a bimodal particle size distribution, the particle diameter d_{L} corresponding to the peak top of the large particles was about 214 μm, and the particle diameter d_{S} corresponding to the peak top of the small particles was about 49 μm. The ratio (frequency corresponding to peak top of small particle/frequency corresponding to peak top of large particle) of the frequency corresponding to the peak top of the small particle to the frequency corresponding to the peak top of the large particle of Composition b was 1.79. Further,

Composition a showed only a single peak at about 70 μm, and Composition c showed only a single peak at about 100 μm. Also, with respect to the average particle diameter and D50, the values of Composition a and Composition b were smaller than the values of Composition c.

### (Experimental Example 4) Bulk Density and Angle of Repose Measurements

For the Compositions a, b, and c obtained in Production Examples 1 to 3, the bulk density and the angle of repose were subjected to measurement by the following method. The results are shown in Table 11.

### (Measurement Method for Bulk Density)

The bulk density of Composition a, b, or c was calculated from the weight obtained when Composition a, b, or c was filled into a 100 mL graduated cylinder.

### (Measurement Method for Angle of Repose)

The angle of reposes of Composition a, b, or c was measured by the pouring method.

TABLE 11

| | Bulk Density | Angle of |
|---|---|---|
| Sample | (g/mL) | Repose (°) |
| Composition c | 0.189 | 47 |
| Composition b | 0.209 | 51 |
| Composition a | 0.195 | 50 |

Compared with Composition a and Composition b, both the bulk density and the angle of repose of Composition c showed smaller values.

### (Experimental Example 5) Solubility Test of Reduced Coenzyme Q10 in Artificial Intestinal Fluid

Artificial intestinal fluid was prepared with the composition shown in Table 12, 20 g of the prepared artificial intestinal fluid was put into a test tube, and 200 mg each of Composition a, Composition b, or Composition c, prepared in Production Examples 1 to 3, were added. After the addition, after performing nitrogen substitution, stirring was performed at 37° C. for a time of 3 hours (1000 rpm). The artificial intestinal fluid after stirring was filtered using filter paper, and 1 mL of ethanol and 1 mL of hexane were added to 1 mL of the filtered artificial intestinal fluid and mixed. 1600 μL of methanol was added to 400 μL of the hexane layer of the upper layer obtained by allowing it to stand for 3 minutes after mixing, and then performed filtration using a filter. The concentrations of reduced coenzyme Q10 (QH concentration) and oxidized coenzyme Q10 (QX concentration) in the liquid after filter filtration were measured using high performance liquid chromatography. The results are shown in Table 13.

TABLE 12

| | Addition |
|---|---|
| Reagent | Amount (g) |
| FeSSIF buffer concentrate | 7.327 |
| PW | 82.75 |
| FeSSIF powder | 1.008 |
| Total | 91.085 |

In Table 12, the FeSSIF buffer concentrate is a buffer concentrate for preparing fed state simulated intestinal fluid, manufactured by Biorelevant.com. PW is purified water, and FeSSIF powder is a powder for preparing fed state simulated intestinal fluid, manufactured by Biorelevant.com. By dissolving FeSSIF powder in FeSSIF buffer concentrate, fed state simulated intestinal fluid (taurocholate: 15 mM, phospholipid: 3.75 mM, sodium: 319 mM, chloride: 203 mM, acetate: 144 mM, pH 5.0) can be reproducibly prepared.

TABLE 13

| | QH | QX | Total Q | |
|---|---|---|---|---|
| | Concen- | Concen- | Concen- | QH |
| | tration | tration | tration | Proportion |
| Sample | (μg/ml) | (μg/ml) | (μg/ml) | (%) |
| Composition c | 25.7 | 97.6 | 123.3 | 20.9% |
| Composition b | 50.0 | 109.1 | 159.1 | 31.4% |
| Composition a | 63.5 | 126.7 | 190.2 | 33.4% |

*Evaluated by total Q concentration (QH + QX), taking into account oxidation of QH during experiment

Composition a and Composition b showed higher values compared with Composition c in each of the reduced coenzyme Q10 concentration, the oxidized coenzyme Q10 concentration, and their total concentration, and were confirmed to exhibit high solubility in artificial intestinal fluid.

### Experiment 2

By the following method, Composition D, Composition E, Composition F, and Composition G were prepared.

Composition D: Composition C and Composition A were mixed at a weight ratio of 2:1.

Composition E: Composition C and Composition A were mixed at a weight ratio of 1.5:1.

Composition F: Composition C and Composition A were mixed at a weight ratio of 1:1.

Composition G: Composition A and Composition B were mixed at a weight ratio of 1:1.

### Production Example 4

Into 1200 g of ethanol, 300 g of Composition D and 122 g of ascorbic acid were added, the mixture was stirred at 79° C., and a reduction reaction was carried out. After 20 hours from the start of the reduction reaction, cooling was performed to 50° C. The ethanol solution after cooling (containing reduced coenzyme Q10) and ethanol were added so that the content of reduced coenzyme Q10 in the solution was 10 wt % and the total amount of the solution was 1700 g, and the mixture was further cooled to 33° C.

After adding a Form I type reduced coenzyme Q10 crystal as a seed crystal to the cooled ethanol solution, and cooling to −1° C., Composition d in solid form containing Form I type reduced coenzyme Q10 was obtained.

### Production Example 5

Except that Composition D in Production Example 4 was changed to Composition E, the procedure was the same as in Production Example 4, and a Composition e in solid form containing Form I type reduced coenzyme Q10 was obtained.

### Production Example 6

Except that Composition D in Production Example 4 was changed to Composition F, the procedure was the same as in Production Example 4, and a Composition f in solid form containing Form I type reduced coenzyme Q10 was obtained.

### Production Example 7

Except that Composition D in Production Example 4 was changed to Composition G, the procedure was the same as in Production Example 4, and a Composition g in solid form containing Form I type reduced coenzyme Q10 was obtained.

### (Experimental Example 6) Component Analysis

Component analysis by high performance liquid chromatography was performed on Compositions d, e, f, and g obtained in Production Examples 4 to 7. Table 14 shows the results. The respective numerical values in Table 14 represent, as calculated from the chromatogram of each composition obtained by high performance liquid chromatography in accordance with Table 1, the peak area ratio of reduced demethoxy Q10 (RD) to reduced coenzyme Q9 (RQ9) (RD/RQ9), the peak area ratio of decaprenylphenol (decaprenyl) to reduced coenzyme Q9 (RQ9) (decaprenyl/RQ9), the peak area ratio of reduced demethoxy Q10 (RD) to reduced coenzyme Q11 (RQ11) (RD/RQ11), the peak area ratio of decaprenylphenol (decaprenyl) to reduced coenzyme Q11 (RQ11) (decaprenyl/RQ11), the peak area ratio of reduced coenzyme Q11 (RQ11) to reduced coenzyme Q9 (RQ9) (RQ11/RQ9), the peak area ratio of reduced coenzyme Q9 (RQ9) to reduced coenzyme Q10 (QH) (RQ9/QH), the peak area ratio of reduced demethoxy Q10 (RD) to reduced coenzyme Q10 (QH) (RD/QH), the peak area ratio of decaprenylphenol (decaprenyl) to reduced coenzyme Q10 (QH) (decaprenyl/QH), the peak area ratio of reduced coenzyme Q11 (RQ11) to reduced coenzyme Q10 (QH) (RQ11/QH), the peak area ratio of decaprenylphenol (decaprenyl) to reduced demethoxy Q10 (RD) (decaprenyl/RD), the peak area ratio of the total of reduced coenzyme Q9 (RQ9), reduced demethoxy Q10 (RD), decaprenylphenol (decaprenyl), and reduced coenzyme Q11 (RQ11) (non-QH) to reduced coenzyme Q10 (QH) (non-QH/QH), the peak area ratio of the total of reduced demethoxy Q10 (RD) and reduced coenzyme Q9 (RQ9) to reduced coenzyme Q10 (QH) ((RD+RQ9)/QH), the peak area ratio of the total of decaprenylphenol (decaprenyl) and reduced coenzyme Q9 (RQ9) to reduced coenzyme Q10 (QH) ((decaprenyl+RQ9)/QH), the peak area ratio of the total of decaprenylphenol (decaprenyl) and reduced coenzyme Q11 (RQ11) to reduced coenzyme Q10 (QH) ((decaprenyl+RQ11)/QH), and the peak area ratio of the total of reduced coenzyme Q9 (RQ9) and reduced coenzyme Q11 (RQ11) to reduced coenzyme Q10 (QH) ((RQ9+RQ11)/QH). In addition, the conditions for high performance liquid chromatography are as follows. (HPLC conditions)
a column: YMC-Pack Pro C18 (manufactured by YMC) 150 mm (length) 4.6 mm (inner diameter),
a mobile phase composition: CH₃OH:C₆H₁₄=9:1 (v/v),
a mobile phase flow rate: 1 mL/min, and
a detection wavelength: 190 nm

TABLE 14

| Peak Area | RD/RQ9 | Decaprenyl/RQ9 | RD/RQ11 | Decaprenyl/RQ11 | RQ11/RQ9 |
|---|---|---|---|---|---|
| Composition c | 0.17 | 0.00 | 0.18 | 0.00 | 0.95 |
| Composition d | 0.35 | 0.52 | 0.21 | 0.30 | 1.72 |
| Composition e | 0.44 | 0.49 | 0.30 | 0.33 | 1.47 |
| Composition f | 0.86 | 0.65 | 0.47 | 0.36 | 1.82 |
| Composition a | 1.00 | 1.44 | 0.30 | 0.43 | 3.37 |
| Composition g | 1.40 | 2.37 | 0.29 | 0.49 | 4.83 |
| Composition b | 2.02 | 3.68 | 0.25 | 0.46 | 8.03 |

| | | | | | | Other | |
|---|---|---|---|---|---|---|---|
| | | | Decaprenyl/ | RQ11/ | Decaprenyl/ | than | (RD + RQ9)/ |
| Peak Area | RQ9/QH | RD/QH | QH | QH | RD | QH/QH | QH |
| Composition c | 0.0056 | 0.00096 | 0.00 | 0.0054 | 0.00 | 0.012 | 0.0066 |
| Composition d | 0.0061 | 0.0022 | 0.0032 | 0.010 | 1.47 | 0.022 | 0.0082 |
| Composition e | 0.0074 | 0.0032 | 0.0036 | 0.011 | 1.12 | 0.025 | 0.011 |
| Composition f | 0.0052 | 0.0045 | 0.0034 | 0.0095 | 0.76 | 0.023 | 0.0097 |
| Composition a | 0.0049 | 0.0049 | 0.0071 | 0.017 | 1.44 | 0.034 | 0.0098 |
| Composition g | 0.0041 | 0.0057 | 0.0096 | 0.020 | 1.70 | 0.039 | 0.0097 |
| Composition b | 0.0026 | 0.0053 | 0.0096 | 0.021 | 1.82 | 0.039 | 0.0079 |

| | (Decaprenyl + RQ9)/ | (Decaprenyl + RQ11)/ | (RQ9 + RQ11)/ |
|---|---|---|---|
| Peak Area | QH | QH | QH |
| Composition c | 0.0056 | 0.0054 | 0.011 |
| Composition d | 0.0093 | 0.014 | 0.017 |
| Composition e | 0.011 | 0.014 | 0.018 |
| Composition f | 0.0087 | 0.013 | 0.015 |
| Composition a | 0.012 | 0.024 | 0.022 |
| Composition g | 0.014 | 0.029 | 0.024 |
| Composition b | 0.012 | 0.031 | 0.024 |

As shown in Table 14, in Compositions d, e, f, and g, the ratio (RD/RQ9) of the peak area of reduced demethoxy Q10 (RD) to the peak area of reduced coenzyme Q9 was 0.3 or more. In particular, in Compositions d, e, f, and g, the value of RD/RQ9 was 0.35 or more, and in Compositions e, f, and g, the value of RD/RQ9 was 0.40 or more, and in Compositions f and g, the value of RD/RQ9 was 0.80 or more, and in Composition g, the value of RD/RQ9 was 1.0 or more.

### (Experimental Example 7) Crystal Polymorphic Stability Test

After 0.5 g of Form II type reduced coenzyme Q10 crystal was added to 5.0 g of each of Compositions a, b, c, d, e, f and g obtained in Production Examples 1 to 7 and containing Form I type reduced coenzyme Q10 crystal, 2 g of 99.5% ethanol was added and the mixture was stirred. After stirring, nitrogen substitution was performed, and it was allowed to stand at 37° C. for 6 hours. Differential scanning calorimetry was performed on the samples obtained before being allowed to stand (0 hours) and after being allowed to stand for 6 hours, and the endothermic peak area due to melting of the Form I crystal and the endothermic peak area due to melting of the Form I crystal were determined. The proportion (%) of the endothermic peak area of the Form II crystal to the total of the endothermic peak area of the Form I crystal and the endothermic peak area of the Form II crystal was calculated as the ratio of the Form II crystal. The results are shown in Table 15.

TABLE 15

| | Composition | Composition | Composition | Composition | Composition | Composition | Composition |
|---|---|---|---|---|---|---|---|
| | c | d | e | f | a | g | b |
| 0 hr | 7.6 | 9.0 | 7.8 | 8.5 | 9.8 | 9.5 | 8.4 |
| 6 hr | 26.4 | 21.2 | 19.6 | 14.6 | 10.3 | 12.1 | 7.5 |

* Numerical values in table indicate Form II type ratio (%).

As shown in Table 15, Compositions a, b, d, e, f, and g having an RD/RQ9 value of 0.3 or more had a small change in the ratio of Form II crystal at the time point of an elapsed time of 6 hours from the start of standing. From this, it was suggested that the stability of the Form I type reduced coenzyme Q10 crystal contained in Compositions a, b, d, e, f, and g is high, and that it is unlikely to undergo transition to Form II crystal. On the other hand, in Composition c, with elapsed time, the ratio of the Form II crystal increased, and the stability of the Form I type reduced coenzyme Q10 crystal was inferior compared with Compositions a, b, d, e, f, and g.

### (Experimental Example 8) Average Particle Diameter Measurement

For Compositions d, e, f, and g obtained in Production Examples 4 to 7, in wet analysis, the particle size distribution on a volume basis was measured by a laser diffraction/scattering particle size distribution analyzer (LA-960, manufactured by HORIBA, Ltd.).

From the obtained particle size distribution, the average particle diameter was obtained. Table 16 shows the results.

TABLE 16

| | Average Particle |
|---|---|
| | Diameter (μm) |
| Composition c | 110 |
| Composition d | 111 |
| Composition e | 76 |
| Composition f | 89 |
| Composition a | 70 |
| Composition g | 62 |
| Composition b | 87 |

With respect to the average particle diameter, the values of Compositions e, f, and g were smaller than the value of Composition d.

### (Experimental Example 9) Solubility Test of Reduced Coenzyme Q10 in Artificial Intestinal Fluid

An artificial intestinal fluid with the composition shown in Table 12 was prepared, 10 g of the prepared artificial intestinal fluid was placed into a test tube, and 1000 mg each of Compositions a to g prepared in Production Examples 1 to 7 were added. After the addition, after performing nitrogen substitution, stirring was performed at 37° C. for a time of 3 h (700 rpm). The artificial intestinal fluid after stirring was filtered using filter paper, and the artificial intestinal fluid after filtration was subjected to centrifugation (9500 rpm, 10 min). 1 mL of ethanol and 1 mL of hexane were added to 1 mL of the supernatant of the artificial intestinal fluid obtained by centrifugation, and mixed. 1600 μL of methanol was added to 400 μL of the hexane layer of the upper layer obtained by allowing it to stand for 3 minutes after mixing, and filtration with a filter was then performed. The concentrations of reduced coenzyme Q10 (QH concentration) and oxidized coenzyme Q10 (QX concentration) in the liquid after filter filtration were measured using high performance liquid chromatography. The results are shown in Table 17.

TABLE 17

| | Total Q Concentration |
|---|---|
| | (μg/mL) |
| Composition c | 82.3 |
| Composition d | 85.8 |
| Composition e | 92.0 |
| Composition f | 91.9 |
| Composition a | 149.1 |
| Composition g | 141.8 |
| Composition b | 154.1 |

Compositions a, b, d, e, f, and g showed higher values than Composition c with respect to the total concentration of reduced coenzyme Q10 and oxidized coenzyme Q10, and it was confirmed that they exhibited high solubility in artificial intestinal fluid.

The upper limit value and/or lower limit value of the numerical ranges described in the present specification can be arbitrarily combined to define preferred ranges. For example, it is possible to define a preferred range by arbitrarily combining upper limit values and lower limit values of numerical ranges, it is possible to define a preferred range by arbitrarily combining upper limit values of numerical ranges with each other, and it is also possible to define a preferred range by arbitrarily combining lower limit values of numerical ranges with each other. Also, in the present application, a numerical range represented using the term “to” includes the numbers described before and after the term “to” as the lower limit value and the upper limit value, respectively.

While this aspect has been described in detail above, the specific configuration is not limited to this aspect, and even if there are design changes within a range that does not depart from the gist of the present disclosure, those are included in the present disclosure.

All publications, patents, and patent applications cited in the present specification are incorporated herein by reference in their entireties.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A reduced coenzyme Q10 composition comprising:
reduced coenzyme Q10,
reduced demethoxy Q10 (RD), and
reduced coenzyme Q9 (RQ9),
wherein a peak area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 in a chromatogram of the composition is 0.3 or more,
wherein the chromatogram is obtained by high performance liquid chromatography under conditions in which
a column is a C18 column (150 mm in length and 4.6 mm in inner diameter),
a mobile phase composition is CH₃OH:C₆H₁₄ at 9:1 (v/v),
a mobile phase flow rate is 1 mL/min, and
a detection wavelength is 190 nm.

2. The reduced coenzyme Q10 composition according to claim 1, wherein the peak area ratio (RD/RQ9) is 1.0 or more.

3. The reduced coenzyme Q10 composition according to claim 1, wherein the peak area ratio (RD/RQ9) is 2.0 to 8.0.

4. The reduced coenzyme Q10 composition according to claim 1, further comprising decaprenylphenol.

5. The reduced coenzyme Q10 composition according to claim 4, wherein a peak area ratio (decaprenylphenol/RQ9) of a peak containing the decaprenylphenol having the relative retention time (RRT) of 1.34 to 1.41 to the peak containing the reduced coenzyme Q9 (RQ9) having the relative retention time (RRT) of 0.69 to 0.73 in the chromatogram of the composition is 0.1 to 10.0.

6. The reduced coenzyme Q10 composition according to claim 5, wherein the peak area ratio (decaprenylphenol/RQ9) is 0.1 to 4.0.

7. The reduced coenzyme Q10 composition according to claim 5, wherein the peak area ratio (decaprenylphenol/RQ9) is 1.0 to 4.0.

8. The reduced coenzyme Q10 composition according to claim 4, further comprising reduced coenzyme Q11 (RQ11), wherein a peak area ratio (decaprenylphenol/RQ11) of a peak containing the decaprenylphenol having the relative retention time (RRT) of 1.34 to 1.41 to a peak containing the reduced coenzyme Q11 (RQ11) having the relative retention time (RRT) of 1.45 to 1.49 in the chromatogram of the composition is 0.01 to 1.5.

9. The reduced coenzyme Q10 composition according to claim 8, wherein the peak area ratio (decaprenylphenol/RQ11) is 0.05 to 0.8.

10. The reduced coenzyme Q10 composition according to claim 8, wherein the peak area ratio (decaprenylphenol/RQ11) is 0.1 to 0.5.

11. The reduced coenzyme Q10 composition according to claim 5, further comprising reduced coenzyme Q11 (RQ11), wherein a peak area ratio (decaprenylphenol/RQ11) of the peak containing the decaprenylphenol having the relative retention time (RRT) of 1.34 to 1.41 to a peak containing the reduced coenzyme Q11 (RQ11) having the relative retention time (RRT) of 1.45 to 1.49 in the chromatogram of the composition is 0.01 to 1.5.

12. The reduced coenzyme Q10 composition according to claim 8, wherein a peak area ratio (RQ11/RQ9) of the peak containing the reduced coenzyme Q11 (RQ11) having the relative retention time (RRT) of 1.45 to 1.49 to the peak containing the reduced coenzyme Q9 (RQ9) having the relative retention time (RRT) of 0.69 to 0.73 in the chromatogram of the composition is 1.0 or more.

13. The reduced coenzyme Q10 composition according to claim 8, wherein the peak area ratio (RQ11/RQ9) is 1.5 or more.

14. The reduced coenzyme Q10 composition according to claim 8, wherein the peak area ratio (RQ11/RQ9) is 2.0 to 10.0.

15. The reduced coenzyme Q10 composition according to claim 11, wherein a peak area ratio (RQ11/RQ9) of the peak containing the reduced coenzyme Q11 (RQ11) having the relative retention time (RRT) of 1.45 to 1.49 to the peak containing the reduced coenzyme Q9 (RQ9) having the relative retention time (RRT) of 0.69 to 0.73 in the chromatogram of the composition is 1.0 or more.

16. The reduced coenzyme Q10 composition according to claim 1, wherein the reduced coenzyme Q10 composition is in solid form.

17. The reduced coenzyme Q10 composition according to claim 1, further comprising an antioxidant.

18. A production method of reduced coenzyme Q10 composition comprising:
separating, from a solution comprising reduced coenzyme Q10, reduced demethoxy Q10 (RD), and reduced coenzyme Q9 (RQ9), a composition in solid form comprising the reduced coenzyme Q10, the reduced demethoxy Q10, and the reduced coenzyme Q9,
wherein a peak area ratio (RD/RQ9) of a peak containing the reduced demethoxy Q10 (RD) having a relative retention time (RRT) relative to a retention time (RT) of a peak top of the reduced coenzyme Q10 of 0.88 to 0.94 to a peak containing the reduced coenzyme Q9 (RQ9) having the relative retention time (RRT) of 0.69 to 0.73 in a chromatogram of the composition is 0.3 or more,
wherein the chromatogram of the composition after the separation is obtained by high performance liquid chromatography under conditions in which
a column is a C18 column (150 mm in length and 4.6 mm in inner diameter),
a mobile phase composition is CH₃OH:C₆H₁₄ at 9:1 (v/v),
a mobile phase flow rate is 1 mL/min, and
a detection wavelength is 190 nm.

19. The production method for the reduced coenzyme Q10 composition according to claim 18,
wherein the separating includes adding a seed crystal of reduced coenzyme Q10 to the solution and cooling the solution.
